(19) United States
(12) Patent Application Publication
(10) Pub. No.: US 2008/0255358 A1
(43) Pub. Date: Oct. 16, 2008

(54) Derivatives of Imidazo [1,2-A] Pyridine Useful as Medicaments For Treating Gastrointestinal Diseases

(76) Inventors: Mark James Bamford, Essex (GB); Emmanuel Hubert Demont, Essex (GB); Richard Leonard Elliott, Essex (GB); Gerard Martin Paul Giblin, Essex (GB); Antoinette Naylor, Essex (GB); Terence Aaron Panchal, Essex (GB); Jason Witherington, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/909,114

(22) PCT Filed: Mar. 22, 2006
(86) PCT No.: PCT/EP2006/002952

§ 371 Date: Sep. 19, 2007

(30) Foreign Application Priority Data
Mar. 24, 2005 (GB) 0506137.9
Apr. 7, 2005 (GB) 0507101.4
Jun. 24, 2005 (GB) 0512923.4
Oct. 19, 2005 (GB) 0521274.1

(51) Int. Cl. C07D 401/14; C07D 471/04; A61P 1/00

(52) U.S. Cl. 544/333; 546/121

(57) ABSTRACT

Disclosed are imidazopyridine compounds having the formula:

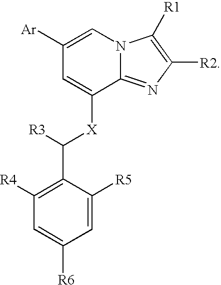

This invention relates to newly identified imidazopyridine compounds, to the use of such compounds in therapy and to their production.

The gastric H⁺/K⁺ ATPase, or proton pump, is responsible for gastric acid secretion from the acid secreting parietal cells of the stomach. The H⁺/K⁺ ATPase actively transports protons and K⁺ ions in opposite directions in a non-electrogenic manner, coupled to the hydrolysis of ATP. Under physiological conditions, this generates and maintains a proton gradient in excess of a million-fold across the luminal membrane of the gastric parietal cell.

Gastric acid is one of the major risk factors for gastrointestinal disease and specific inhibitors of the gastric H⁺/K⁺ ATPase are currently used for clinical treatments and control of hyperacidity. Such inhibitors can be classified into two groups, the first of which are the irreversible inhibitors such as omeprazole, which are termed proton pump inhibitors or PPIs. This class of compounds are weak bases which accumulate in the acidic canaliculi of active parietal cells where they rapidly form cationic tetracyclic sulphenamides. The sulphenamide then binds irreversibly to the lumenal surface of the H⁺/K⁺ ATPase and inhibits its activity.

In patients with gastro-oesophageal reflux disease (GERD), PPIs are currently the treatment of choice. However, the majority of patients treated with PPIs, histamine H2 receptor antagonists or prokinetic agents continue to experience frequent heartburn and nocturnal acid breakthrough, suggesting that current therapies may not always achieve sufficient control of acid production.

Currently available PPIs may take 3-5 days to achieve maximal acid inhibition due to the fact that they require activation within the acidic canaliculus and thus target only actively secreting parietal cells. A proportion of the pumps therefore remains un-inhibited after each dose, and repeated daily dosing is required to reach a steady-state of inhibition.

The second group of H⁺/K⁺ ATPase inhibitors are the reversible inhibitors, which are described as acid pump antagonists (APAs) or potassium-competitive acid blockers (p-CABs). In contrast to the currently available PPIs, the reversible, K⁺ competitive APAs do not require activation in an acidic environment and block acid secretion in a direct manner by binding at or near the potassium binding site, resulting in a very rapid onset of action compared to PPIs. It is also expected that APAs will afford improvements in control of acid secretion over an extended period.

Accordingly the present invention provides a compound of formula (I)

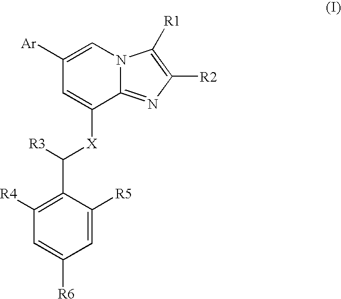

wherein X is NH, NR7 or O;
R1 is H, C₁₋₄ alkyl, CH₂CN, CH₂NH₂, C₃₋₆cycloalkyl, C₃₋₆cycloalkylC₁₋₄alkyl, C₁₋₄alkoxy, C₂₋₆alkenyl, C₂₋₆alkenyloxyC₁₋₄alkyl, C₂₋₆alkynyl, hydroxyC₁₋₄alkyl, C₁₋₄alkoxyC₁₋₄alkyl, hydroxyC₁₋₄alkoxyC₁₋₄alkyl, fluoroC₁₋₄alkyl, C₂₋₆alkynyloxyC₁₋₄alkyl, C₁₋₄alkylsulphonylC₁₋₄alkyl or NR8R9, where each of R8 and R9, which may be the same or different, are H or C₁₋₄alkyl or, together with the nitrogen to which they are attached, form a 5- or 6-membered heterocyclic group containing 0 to 3 further heteroatoms selected from N, O and S;
R2 is C₁₋₄alkyl, NH₂, C₃₋₆cycloalkyl, C₃₋₆cycloalkylC₁₋₄alkyl, C₁₋₄alkoxy, C₂₋₆alkenyl, hydroxyC₁₋₄alkyl, C₁₋₄alkoxyC₁₋₄alkyl, hydroxyC₁₋₄alkoxyC₁₋₄alkyl, cyanoC₁₋₄alkyl, haloC₁₋₄alkyl or aminocarboxyC₁₋₄alkyl;
R3 is H or C₁₋₄alkyl;
R4 and R5, which may be the same or different, are H, C₁₋₄alkyl, OH, halogen, C₁₋₄alkoxy, NR14R15 where each of R14 and R15, which may be the same or different, are H or C₁₋₄alkyl, NHCONR10R11 or OCONR10R11 where each of R10 and R11, which may be the same or different, are H or C₁₋₄alkyl or, together with the nitrogen to which they are attached, form a 5- or 6-membered heterocyclic group containing 0 to 3 further heteroatoms selected from N, O and S;
or R3 and R4 together with the interconnecting atoms form a 5- or 6-membered carbocyclic group or a heterocyclic group containing 1 heteroatom selected from N, O and S, which carbocyclic or heterocyclic group is optionally substituted with one group selected from C₁₋₄alkyl, OH, OC₁₋₄alkyl, halogen and NR16R17 where each of R16 and R17, which may be the same or different, are H or C₁₋₄alkyl;
R6 is H, C₁₋₄alkyl, halogen, OH, NHCO₂C₁₋₄alkyl, NR18R19 where each of R18 and R19, which may be the same or different, are H or C₁₋₄alkyl;
R7 is C₁₋₄alkyl;
or R4 and R7 together with the interconnecting atoms form a 5- to 7-membered heterocyclic group containing 0 or 1 further heteroatoms selected from N, O and S, which heterocyclic group is optionally substituted with one group selected from C₁₋₄alkyl, OH, OC₁₋₄alkyl, halogen and NR20R21 where each of R20 and R21, which may be the same or different, are H or C₁₋₄alkyl;
and
Ar is aryl, or a 5- or 6-membered monocyclic, or a 7- to 12-membered bicyclic, heteroaryl group (containing 1 to 4 heteroatoms selected from N, O and S) which aryl or heteroaryl group is independently optionally substituted with 1, 2, 3 or 4 groups selected from the group consisting of halogen, hydroxy, oxo, cyano, C₁₋₄alkyl, C₁₋₄alkoxy, hydroxyC₁₋₄alkyl, hydroxyC₁₋₄alkoxy, haloC₁₋₄alkyl, haloC₁₋₄alkoxy, arylC₁₋₄alkoxy, C₁₋₄alkylthio, C₁₋₄alkoxyC₁₋₄alkyl, C₃₋₆cycloalkylC₁₋₄alkoxy, C₁₋₄alkanoyl, C₁₋₄alkylsulfonyl, C₁₋₄alkylsulfonyloxy, C₁₋₄alkylsulfonylC₁₋₄alkyl, arylsulfonyl, arylsulfonyloxy, aryloxysulfonyl, arylsulfonylC₁₋₄alkyl, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl, arylcarboxamidoC₁₋₄alkyl, arylaminosulfonyl, arylaminocarbonyl, aroyl, aroylC₁₋₄alkyl, arylC₁₋₄alkanoyl, NR12R13 and CONR12R13, where each of R12 and R13, which may be the same or different, are H, C₁₋₄ alkyl, hydroxyC₁₋₄alkyl, alkoxyC₁₋₄alkyl, C₃₋₆cycloalkyl, C₃₋₆cycloalkylC₁₋₄alkyl or, together with the nitrogen to which they are attached, form a 5- or 6-membered heterocyclic group containing a further heteroatom selected from N, O or S;
or a pharmaceutically acceptable salt thereof;
with the proviso that the compound is not:
1-(8-{[(4-fluorophenyl)methyl]oxy}-2-methylimidazo[1,2-a]pyridin-6-yl)-2(1H)-pyridinone.

Examples of Ar include a monocyclic aryl, for example phenyl; a bicyclic aryl, for example napthyl; a monocyclic heteroaryl for example pyridinyl, pyrimidinyl, thiazolyl, pyridazinyl, pyrazinyl, oxazolyl, triazolyl, tetrazolyl, imidazolyl, pyrazolyl, furanyl, thienyl, pyrrolyl, oxadiazolyl, thiadiazolyl, triazinyl, isothiazolyl and isoxazolyl; or a bicyclic heteroaryl, for example quinoxalinyl, quinazolinyl, pyridopyrazinyl, benzoxazolyl, benzimidazolyl, naphthyridinyl, quinolinyl, benzofuranyl, benzothienyl, indolyl, benzothiazolyl, oxazolyl[4,5-b]pyridiyl, pyridopyrimidinyl or isoquinolinyl.

The Ar group may be attached to the imidazopyridine core through either a carbon atom or a heteroatom of the Ar ring. For example if the Ar group is an imidazolyl group, the bond to the imidazopyridine core may be through either a carbon atom (C-linked) or a nitrogen atom (N-linked) on the imidazolyl Ar group.

When used herein the term aryl means a 5- or 6-membered aromatic ring for example phenyl, or a 7 to 12 membered bicyclic ring system where at least one of the rings is aromatic for example naphthyl or indenyl. Aroyl is to be interpreted accordingly.

When the compound contains a C₁₋₄alkyl group, whether alone or forming part of a larger group, e.g. C₁₋₄alkoxy or C₁₋₄alkylthio, the alkyl group may be straight chain, branched or cyclic, or combinations thereof. Examples of C₁₋₄alkyl are methyl or ethyl. An example of C₁₋₄alkoxy is methyloxy.

Halogen or “halo” (when used, for example, in haloC₁₋₄alkyl) means fluoro, chloro, bromo or iodo.

In one embodiment Ar is selected from phenyl, pyridinyl, pyrimidinyl, pyridazinyl, pyrazinyl, oxazolyl, furanyl, oxadiazolyl, isoxazolyl, pyrazolyl, triazolyl, tetrazolyl, imidazolyl and thienyl.

In a further embodiment the optional substituents for Ar are C₁₋₄alkyl, C₁₋₄alkoxy, carboxamide, oxo, cyano, amino or hydroxyC₁₋₄alkyl.

In one embodiment Ar is pyridinyl or substituted pyridinyl. Substituted pyridinyl may, for example, be methylpyridinyl, methyloxypyridinyl, carboxamidopyridinyl, aminopyridinyl, pyridinonyl, methylpyridinonyl cyanopyridinonyl or chloropyridinonyl.

In a further embodiment Ar is selected from pyrazolyl, triazoyl, tetrazolyl, pyrimidinyl, pyridazinyl and furanyl. In this embodiment substituted Ar groups include pyrazolonyl, methylpyrazolyl, hydroxymethylpyrazolyl, pyrimidinonyl, pyrimidinedionyl, dimethylpyrimidinedionyl and pyridazinonyl.

In one embodiment X is NH or O.

In one embodiment X is NH.

In one embodiment X is O.

In one embodiment R1 is C₁₋₄ alkyl, CH₂CN, C₁₋₄alkoxy, hydroxyC₁₋₄alkyl or C₁₋₄alkoxyC₁₋₄alkyl.

In one embodiment R2 is C₁₋₄alkyl.

In a further embodiment R1 and R2 are both methyl.

In one embodiment R3 is H.

In one embodiment R4 is H, C₁₋₄alkyl or halogen.

In one embodiment R5 is H, C₁₋₄alkyl or halogen.

In a further embodiment R4 and R5 are not both H.

In one embodiment R6 is H, C₁₋₄alkyl or halogen.

In a further embodiment R4 and R5 are both methyl and R6 is H.

In a still further embodiment R4 is methyl, R5 is ethyl and R6 is H.

In a further embodiment X is NH or O; R3 is H; R1,R2, R4 and R5 are all methyl; and R6 is H.

In a still further embodiment X is NH or O; R3 is H; R1, R2, R4 and R5 are all methyl; R6 is H; and Ar is pyridinonyl, pyrazolyl or triazolyl.

In one embodiment R6 is fluoro.

In one embodiment one of R4 and R5 is fluoro, chloro or bromo and the other is H. In a further embodiment R4 and R5 are both fluoro.

In a further embodiment X is NH or O; R3 is H; R1 and R2 are both methyl; R6 is H or fluoro; one of R4 and R5 is chloro and the other is H.

In a still further embodiment X is NH or O; R3 is H; R1 and R2 are both methyl; R6 is H or fluoro; one of R4 and R5 is chloro and the other is H; and Ar is pyridinonyl, pyrazolyl or triazolyl.

In a yet further embodiment X is NH or O; R3 is H; R1 and R2 are both methyl; R6 is fluoro; and R4 and R5 are both methyl.

In a still further embodiment X is NH or O; R3 is H; R1 and R2 are both methyl; R6 is fluoro; R4 and R5 are both methyl; and Ar is pyridinonyl, pyrazolyl or triazolyl.

It is to be understood that the present invention covers all combinations of particularized groups and substituents described herein above.

It will be appreciated that compounds of formula (I) may exist as R or S enantiomers. The present invention includes within its scope all such isomers, including mixtures. Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms and mixtures thereof.

It will be understood that the invention includes pharmaceutically acceptable derivatives of compounds of formula (I) and that these are included within the scope of the invention.

Particular compounds according to the invention include those mentioned in the examples and their pharmaceutically acceptable derivatives.

As used herein “pharmaceutically acceptable derivative” includes any pharmaceutically acceptable salt, ester or salt of such ester of a compound of formula (I) which, upon administration to the recipient is capable of providing (directly or indirectly) a compound of formula (I) or an active metabolite or residue thereof.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse, J. Pharm. Sci. (1977) 66, pp 1-19. Such pharmaceutically acceptable salts include acid addition salts formed with inorganic acids e.g. hydrochloric, hydrobromic, sulphuric, nitric or phosphoric acid; and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

The subject invention also includes isotopically-labeled compounds which are identical to those recited in formula (I) and following, but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as 3H, 11C, 14C, 18F, 123I, 125I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as 3H or 14C have been incorporated, are useful in drug and/or substrate tissue distribution assays. Tritiated, ie. 3H, and carbon-14, ie. 14C, isotopes are particularly preferred for their ease of preparation and detectability. 11C and 18F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

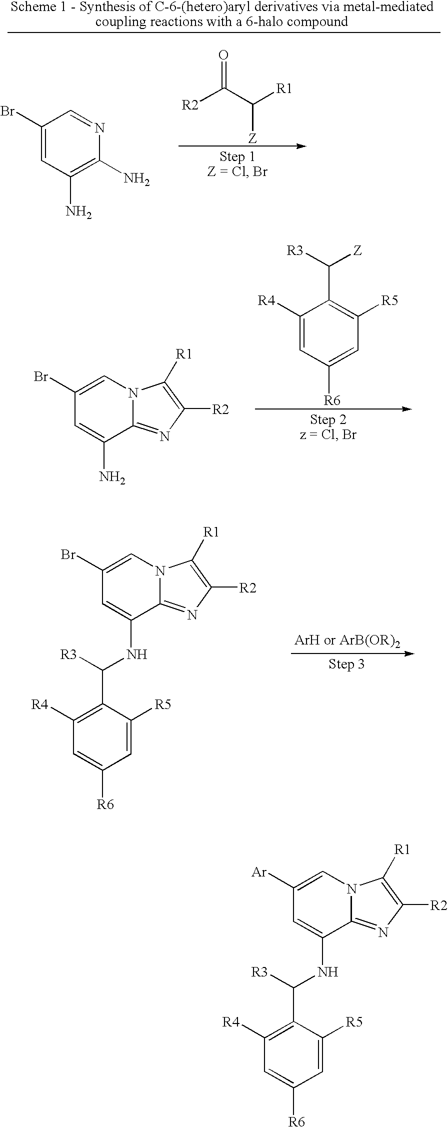

Thus, compounds of the invention for which X is NH can be synthesized as outlined in Scheme 1. Step 1 typically comprises reacting a diamino-halopyridine derivative with the appropriate haloketone in an appropriate solvent such as N-methylpyrrolidinone (NMP) under microwave conditions at an appropriate temperature such as 180° C. for an appropriate time such as 1 h. Alternatively, step 1 can be effected by heating at reflux in ethanol, or by heating at a suitable temperature in NMP. Step 2 consists of reacting the 8-amino-6-haloimidazopyridine with an appropriate benzyl halide such as the benzyl chloride in the presence of a base such as sodium carbonate in a suitable solvent such as dimethylformamide (DMF) for a suitable time such as 3-16 h. Additives such as potassium iodide may be used. In step 3, an appropriate metal-mediated coupling of an aryl group can be used. For example, where the product has Ar linked through nitrogen, Ullman-type couplings can be used, in which the 6-halo compound can be reacted in the presence of copper (I) iodide and a base such as potassium carbonate in a suitable solvent such as dioxane at a suitable temperature such as reflux for a suitable time such as 3 days.

Alternatively, the reaction can be conducted under microwave conditions in a suitable solvent such as DMF or NMP at suitable temperatures up to 195° C. Additives such as trans1,2-diaminocyclohexane may be used, and the base can alternatively be potassium phosphate. When the product has Ar linked through a carbon atom, step 3 consists of reacting the 6-bromo-intermediate with an appropriate arylboronic acid or arylboronate ester under Suzuki-type conditions in the presence of an appropriate palladium coupling reagent such as [1,1′-bis(diphenylphosphino)ferrocene]dichloropalladium in the presence of a suitable base such as sodium carbonate in a suitable solvent system such as water/DMF at a suitable temperature such as 100° C. for a suitable time such as 1 h. This reaction, also, may alternatively be conducted under microwave conditions.

Alternatively, the 6-halo intermediate can be converted to the corresponding boronic acid or boronate ester prior to reaction with the appropriate aryl halide as in scheme 2:

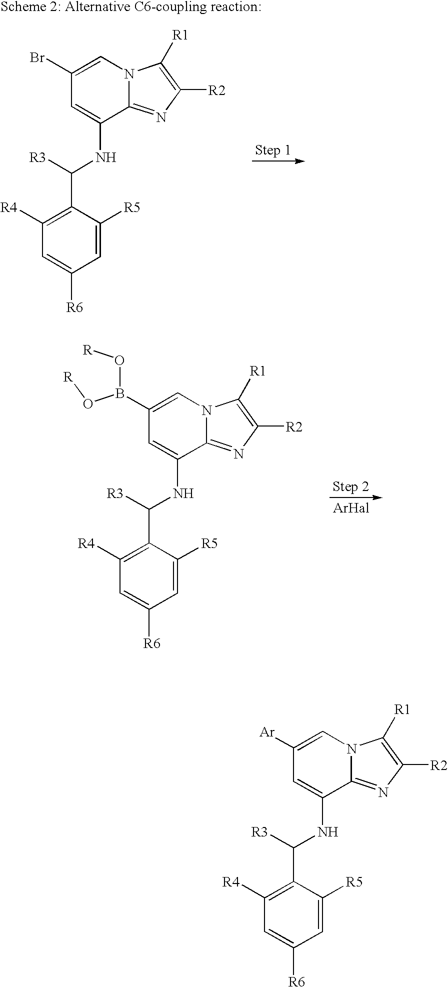

Typically, step 1 consists of reacting the 6-halo-derivative with diboron pinacol ester in a suitable solvent such as dioxane in the presence of a suitable palladium reagent such as [1,1′-bis(diphenylphosphino)ferrocene]dichloropalladium and a suitable base such as potassium acetate at a suitable temperature such as reflux. Step 2 typically consists of reaction of this boron derivative with the appropriate aryl halide such as the arylbromide under Suzuki-type conditions.

Alternatively, where the product has Ar linked through a nitrogen atom, step 2 can be effected by reacting the boron derivative with an appropriate N-heterocycle under Chan-Lam-type conditions. Typically, reactions are carried out in the presence of copper acetate and a tertiary amine in a suitable solvent such as dichloromethane at a suitable temperature such as room temperature.

Alternatively, compounds of the invention wherein X is NH can be generated according to Scheme 3:

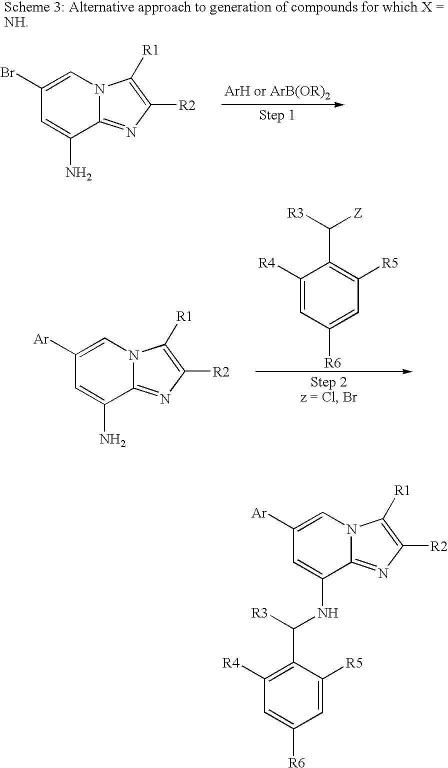

Typically, in step 1 an appropriate metal-mediated coupling of an aryl group can be used. For example, where the product has Ar linked through nitrogen, Ullman-type couplings can be used, in which the 6-halo compound can be reacted in the presence of copper(I) iodide and a base such as potassium carbonate in a suitable solvent such as dioxane at a suitable temperature such as reflux for a suitable time such as 3 days. This may alternatively be conducted under microwave conditions, for example in DMF at a suitable temperature such as 160° C. or in NMP at a suitable temperature such as 190° C. Additives such as trans1,2-diaminocyclohexane maybe used, and the base may alternatively be for example potassium phosphate. When the product has Ar linked through a carbon atom, step 1 consists of reacting the 6-bromo-intermediate with an appropriate arylboronic acid or arylboronate ester under Suzuki-type conditions such as in the presence of an appropriate palladium coupling reagent such as [1,1′-bis(diphenylphosphino)ferrocene]dichloropalladium in the presence of a suitable base such as sodium carbonate in a suitable solvent system such as water/DMF at a suitable temperature such as 100° C. for a suitable time such as 1 h. Step 2 typically consists of reacting the product of step 1 with an appropriate benzyl halide such as the benzyl bromide in the presence of a base such as sodium carbonate in a suitable solvent such as DMF for a suitable time such as 3 h. Additives such as potassium iodide may be used.

Compounds of the invention for which X is O can be synthesized according to steps 1 to 3 of scheme 4:

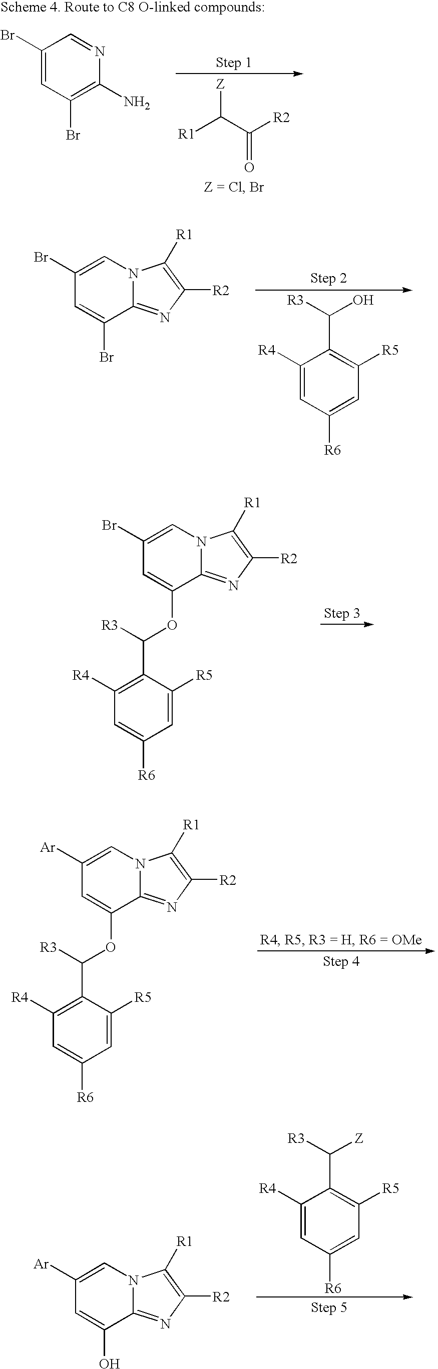

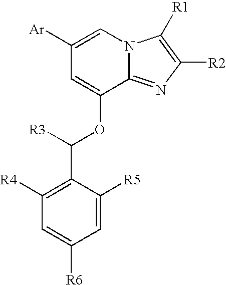

Step 1 typically comprises the use of an appropriate ketone such as alpha-chloro or alpha-bromo ketone, in the presence of a suitable solvent such as NMP, at a suitable temperature such as between 160° C. and 180° C. in the presence of microwaves. Step 2 typically comprises the use of the appropriate benzylic alkoxide (generated by the use of an appropriate base such as sodium hydride, in the presence of a suitable solvent such as DMF at a suitable temperature such as 0° C. to room temperature) in the presence of a suitable solvent such as DMF at an appropriate temperature such as between 60° C. and 90° C. Step 3 typically comprises the use of the appropriate aromatic (Ar) derivative in the presence of an appropriate catalyst such as CuI and an appropriate base such as K₂CO₃ in the presence of a suitable solvent such as NMP or DMF at a suitable temperature such as between 150° C. and 190° C. in the presence of microwaves.

Alternatively, compounds of the invention for which X is O can be generated according to steps 4 and 5 of scheme 4. Thus, when the groups R4, R5, R3 or R6 facilitate removal of the O-benzyl group, for example when R4, R5, R3 are H and R6 (in this scheme) is OMe, the product of step 3 can be subjected to steps 4 and 5 to provide alternative C8-modified analogues. Typically step 4 consists of treating with an acid such as trifluoroacetic acid in a suitable solvent such as dichloromethane, at a suitable temperature such as room temperature. Typically step 5 consists of treating with a suitable base such as sodium hydride in a suitable solvent such as DMF, followed by reacting with the appropriate benzylhalide, such as a benzylbromide, at a suitable temperature such as room temperature for an appropriate time, such as 16 h.

Certain examples of the invention, such as those for which Ar is an oxadiazole, can be generated from the appropriate 6-carboxylic acid derivatives as outlined in Scheme 5:

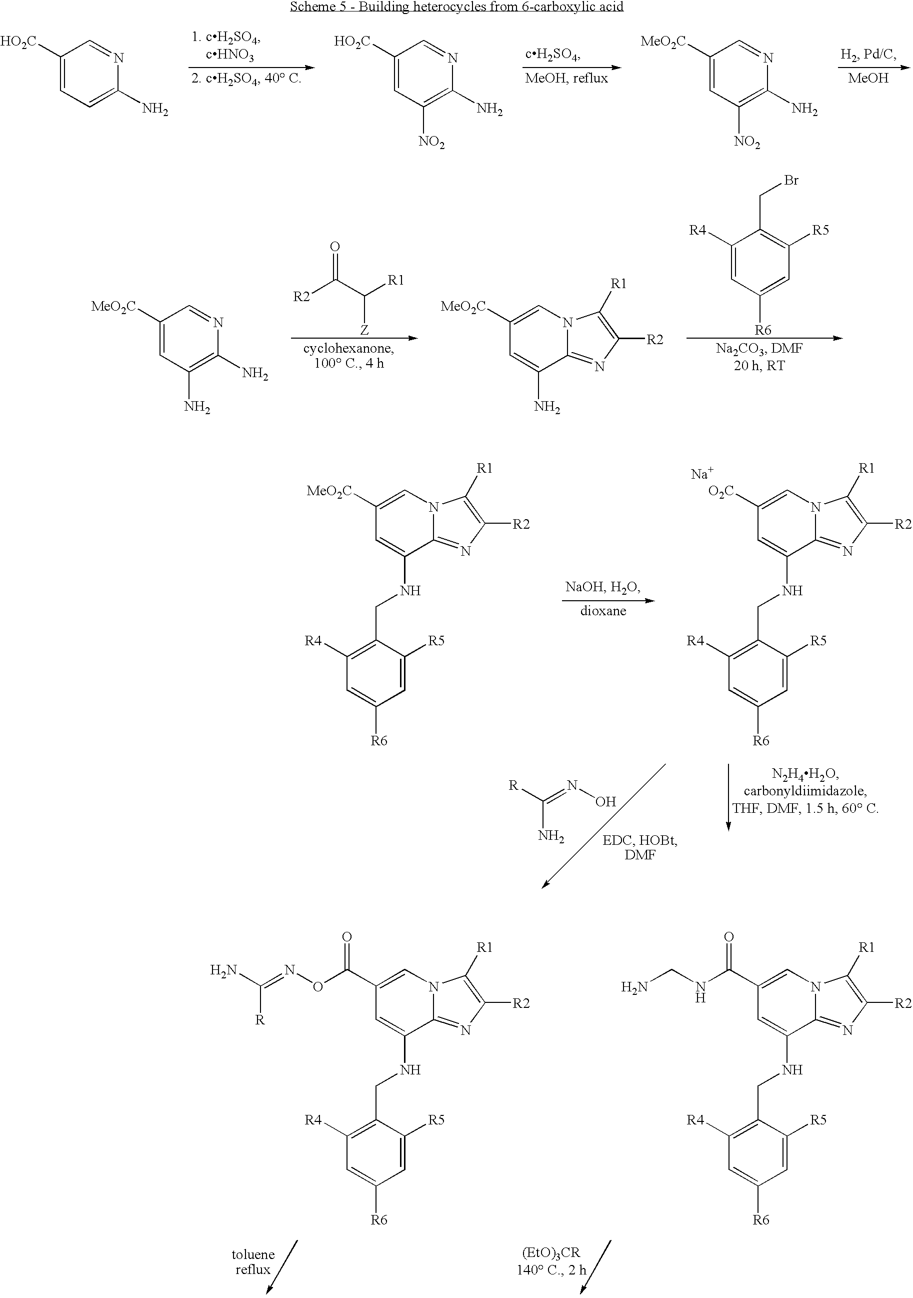

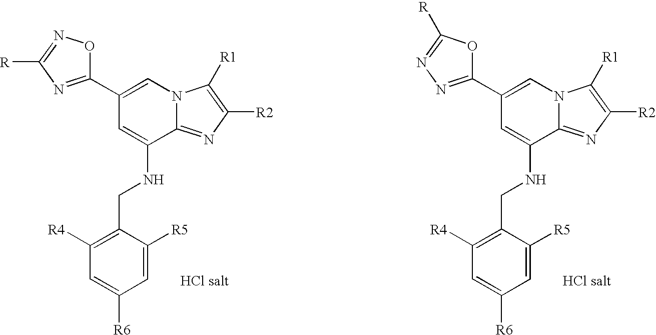

Similarly, certain compounds of the invention, for example those for which Ar is a tetrazole or a triazole, can be generated from the 6-carboxamide derivative as in scheme 6.

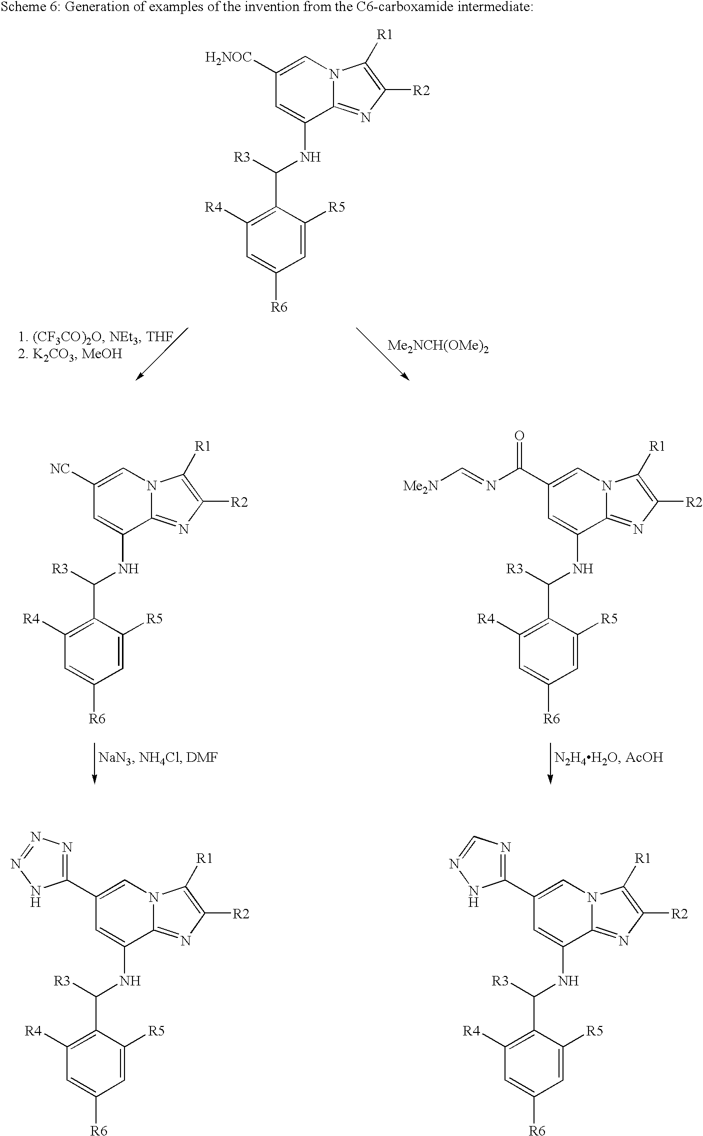

Furthermore, certain compounds of the invention can be derived from the C6-alkynyl derivatives according to scheme 7.

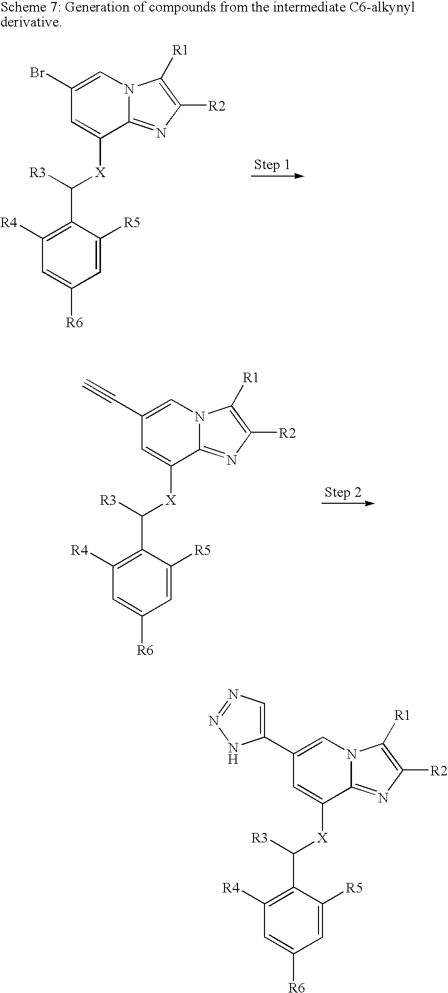

Typically, step 1 consists of firstly reacting the 6-halo derivative, such as the 6-bromo compound, under Sonogashira-type conditions, eg with (trimethylsilyl)acetylene in a suitable solvent such as triethylamine in the presence of appropriate coupling reagents such as Cu(I)iodide and bis(triphenylphosphine)palladium(II) chloride at an appropriate temperature such as 45° C. for an appropriate time, such as 16 h. This is then treated with a base such as potassium hydroxide. Step 2 typically consists of reacting the acetylene intermediate with an appropriate azide source such as (trimethylsilyl)azide in the presence of a suitable reagent such as Cu(I)iodide in a suitable solvent system such as DMF/methanol at a suitable temperature such as 100° C. for a suitable time such as 16 h.

Compounds of the invention for which X is NH and R1 is other than CH₃, for example, CH₂OH, can be generated from compounds of the invention for which X is NH and R1 is H according to procedures such as that in Scheme 8:

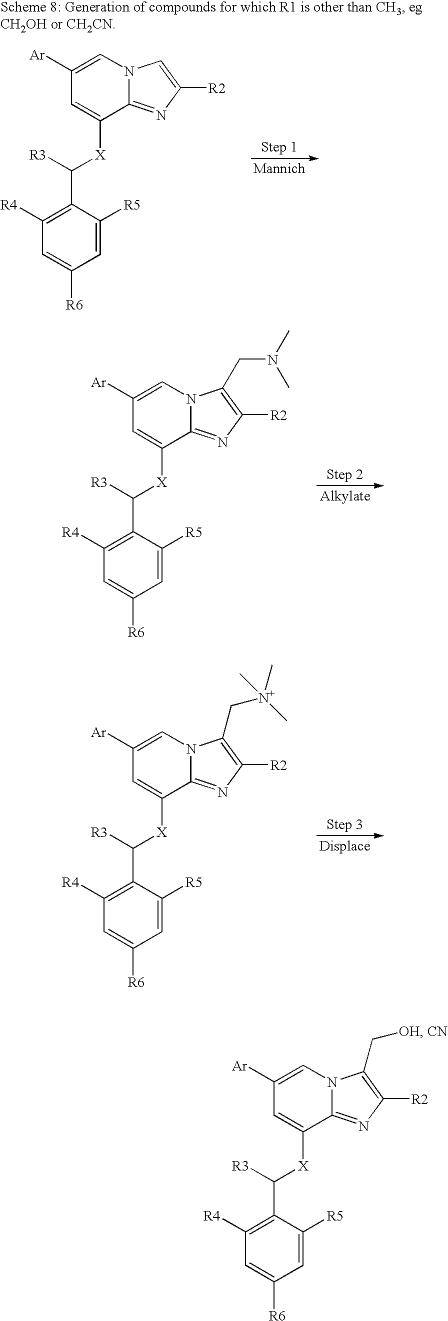

Typically step 1 consists of reacting with a source of formaldehyde such as paraformaldehyde, and a secondary amine salt such as dimethylamine hydrochloride in a suitable solvent such as methanol at a suitable temperature such as reflux. Step 2 consists typically of reacting with an alkylating agent such as iodomethane in a suitable solvent such as ethanol, at a suitable temperature such as ambient temperature. Typically step 3 consists of reacting with a suitable nucleophile such as sodium hydroxide in a suitable solvent such as water/tetrahydrofuran at a suitable temperature such as reflux. Alternatively, step 3 may consist of reaction with a suitable nucleophile such as sodium cyanide, in a suitable solvent such as DMF, at a suitable temperature such as 100° C.

Alternatively, compounds of the invention for which X is O, and R1 is other than CH₃, eg CH₂OH, can be generated according to the general method shown in scheme 9.

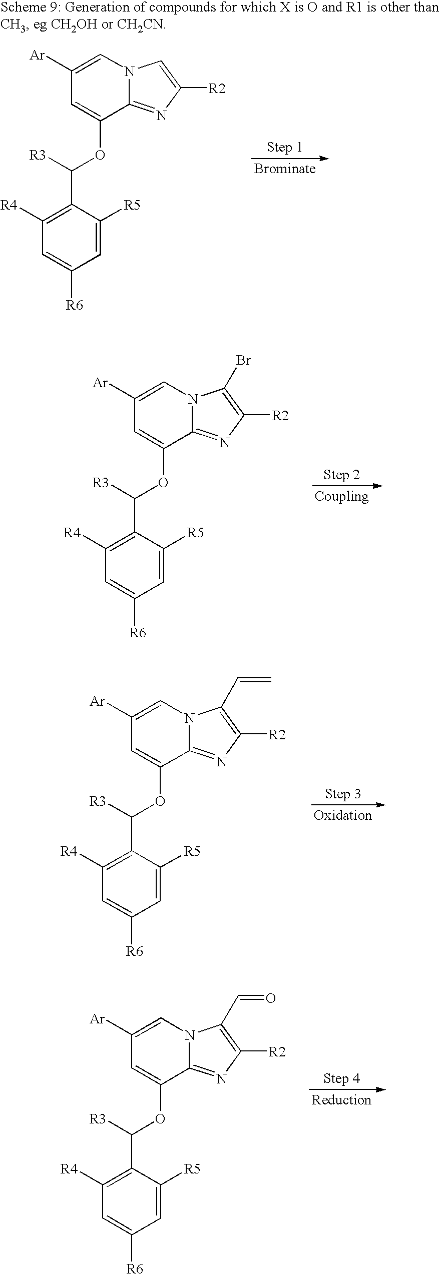

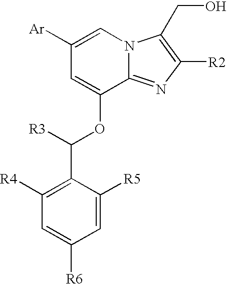

Typically, Step 1 consists of treatment in a suitable solvent such as ethanol, at a suitable temperature such as 0° C. with a brominating agent such as N-bromosuccinimide. Step 2 involves treatment in a suitable solvent such as toluene/ethanol at a suitable temperature such as room temperature with a suitable palladium complex such as tetrakis(triphenylphosphine)palladium(0) in the presence of a suitable base such as potassium carbonate, followed by addition of a suitable boronate derivative such as triethenylboroxin pyridinium complex and heating at a suitable temperature such as at reflux. Step 3 typically involves stirring with a suitable oxidant such as sodium periodate and osmium tetroxide in a suitable solvent such as tetrahydrofuran/water at a suitable temperature such as room temperature. Step 4 typically consists of reduction with a suitable hydride reagent such as sodium borohydride in a suitable solvent such as methanol, at a suitable temperature such as 0° C.

Compounds of the invention for which X is NH or NR7 can alternatively be synthesized according to steps 1 to 3 of scheme 10:

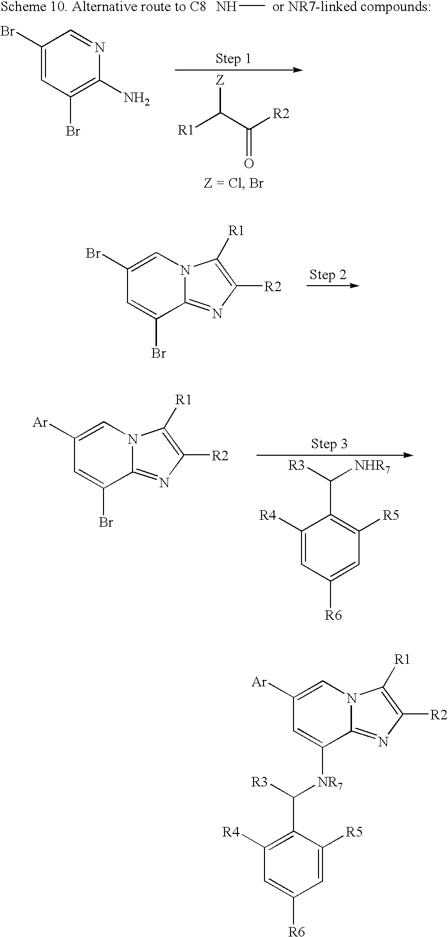

Step 1 typically comprises the use of an appropriate ketone such as alpha-chloro or alpha-bromo ketone, in the presence of a suitable solvent such as NMP, at a suitable temperature such as between 160° C. and 180° C. in the presence of microwaves. Step 2 typically comprises the use of the appropriate aryl substituent, such as triazole, in the presence of a suitable transition metal salt such as Copper(I)iodide and base such as cesium carbonate and complexing agent such as trans 1,2-diaminocyclohexane, in the presence of a suitable solvent such as DMF at an appropriate temperature such as 120° C. optionally under microwave conditions. Step 3 typically comprises the use of the appropriate amine in the presence of a suitable phosphine ligand such as 2-dicyclohexylphosphino-2-(N,N-dimethylamino)biphenyl and palladium complex, such as tris(dibenzylideneacetone)dipalladium (0) and base such as sodium t-butoxide in a suitable solvent such as dioxane and heated under microwave conditions at a suitable temperature such as 120° C.

It will be understood by those skilled in the art that certain compounds of the invention can be converted into other compounds of the invention according to standard chemical methods.

The starting materials for use in Schemes 1 to 10 are commercially available, known in the literature or can be prepared by known methods.

The compounds of formula (I) may be prepared singly or as compound libraries comprising at least 2, e.g. 5 to 1000, preferably 10 to 100 compounds of formula (I). Compound libraries may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I), or pharmaceutically acceptable derivatives thereof.

Pharmaceutically acceptable salts may be prepared conventionally by reaction with the appropriate acid or acid derivative.

The present invention provides compounds of formula (I) and their pharmaceutically acceptable derivatives for use in human or veterinary medicine.

The compounds of formula (I) and their pharmaceutically acceptable derivatives are useful for the treatment of diseases or disorders where an acid pump antagonist (APA) is required such as gastrointestinal diseases or disorders, for example those associated with hyperacidity. The compounds of the invention may be particularly useful for the treatment or prophylaxis of inflammatory gastrointestinal diseases and diseases associated with an imbalance in gastric acid such as gastric or duodenal ulcer, gastritis, gastro-oesophageal reflux disease (GERD), and Zoller-Ellison Syndrome or diseases and disorders where gastric antisecretory effect is desirable for example in patients with gastrinomas and acute upper gastrointestinal bleeding.

The invention also provides a method of treating or preventing diseases or disorders where an antagonist of a human acid pump is required, for example those diseases and disorders mentioned hereinabove, which comprises administering to a subject in need thereof an effective amount of a compound of formula (I), or a pharmaceutically acceptable derivative thereof.

The invention also provides a compound of formula (I), or a pharmaceutically acceptable derivative thereof, for use in the treatment or prophylaxis of diseases or disorders where an antagonist of a human acid pump is required, for example those diseases and disorders mentioned hereinabove.

The invention also provides the use of a compound of formula (I), or a pharmaceutically acceptable derivative thereof, in the manufacture of a medicament for the treatment or prophylaxis of diseases or disorders where an antagonist of a human acid pump is required such as inflammatory gastrointestinal diseases and diseases associated with an imbalance in gastric acid such as gastric or duodenal ulcer, gastritis, gastro-oesophageal reflux disease (GERD), and Zoller-Ellison Syndrome or diseases and disorders where gastric antisecretory effect is desirable for example in patients with gastrinomas and acute upper gastrointestinal bleeding.

For use in therapy the compounds of the invention are usually administered as a pharmaceutical composition. The invention also provides a pharmaceutical composition comprising a compound of formula (I), or a pharmaceutically acceptable derivative thereof, and a pharmaceutically acceptable carrier.

The compounds of formula (I) and their pharmaceutically acceptable derivatives may be administered by any convenient method, e.g. by oral, parenteral, buccal, sublingual, nasal, rectal or transdermal administration, and the pharmaceutical compositions adapted accordingly.

The compounds of formula (I) and their pharmaceutically acceptable derivatives which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilized and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomizing device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomizers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

The dose of the compound of formula (I), or a pharmaceutically acceptable derivative thereof, used in the treatment or prophylaxis of the abovementioned disorders or diseases will vary in the usual way with the particular disorder or disease being treated, the weight of the subject and other similar factors. However, as a general rule, suitable unit doses may be 0.05 to 1000 mg, more suitably 0.05 to 500 mg. Unit doses may be administered more than once a day for example two or three times a day, so that the total daily dosage is in the range of about 0.01 to 100 mg/kg; and such therapy may extend for a number of weeks or months. In the case of pharmaceutically acceptable derivatives the above figures are calculated as the parent compound of formula (I).

No toxicological effects are indicated/expected when a compound of formula (I) is administered in the above mentioned dosage range.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of pharmacologically active compounds of the invention. The Descriptions 1 to 56 illustrate the preparation of intermediates to compounds of the invention.

Where so indicated in the experimental section MDAP represents Mass Directed Auto Prep., an automated system for compound purification by preparative HPLC with detection and collection by desired mass through use of a mass spectrometer in combination with a preparative HPLC system. In the present case a Waters FractionLynx MDAP system was employed with an appropriate reverse phase column using a water/acetonitrile gradient, both solvents containing 0.1% formic acid. Where so indicated in the experimental section microwave heating was performed in Biotage Initiator 60 or Personal Chemistry Optimizer instruments. These instruments allowed the control of temperature up to 250° C. and allowed pressures up to 20 bar with microwave radiation up to 300 W at 2.45 GHz.

### Description 1—6,8-Dibromo-2,3-dimethylimidazo[1,2-a]pyridine

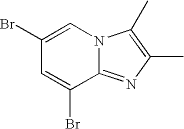

A mixture of 2-amino-3,5-dibromopyridine (5.45 g, 21.6 mmol), 3-bromo-2-butanone (3.64 g, 24.1 mmol) and N-methylpyrrolidinone (11 mL) was heated in an Initiator™ Microwave Synthesizer at 180° C. for 1 hour. After cooling, methanol (10 mL) was added and the mixture poured into ethyl acetate and aqueous sodium bicarbonate. The mixture was filtered, the filtrate layers were separated and the aqueous layer extracted with ethyl acetate. The combined organic extracts were washed twice with water, then brine, dried (MgSO₄) and evaporated. Purification by chromatography on silica gel (ethyl acetate/toluene) gave the title compound. MS (ES+ve): [M+H]⁺ at m/z 303 (C₉H₈⁷⁹Br₂N₂ requires [M+H]⁺ at m/z 303).

### Description 2—6-Bromo-8-{[(2,6-dimethylphenyl)methyl]oxy}-2,3dimethylimidazo[1,2-a]pyridine

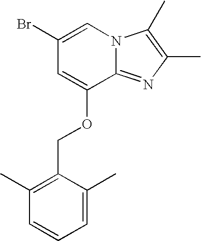

Sodium hydride (228 mg, 60% dispersion in oil, 5.7 mmol) was washed with hexane (2×3 mL), and then dimethylformamide (5 mL) was added. The suspension was cooled in an ice bath, and then a solution of (2,6-dimethylphenyl)methanol (0.739 g, 5.43 mmol) in dimethylformamide (2 mL) was added over 10 minutes. The mixture was then stirred at room temperature for 1 hour. A solution of 6,8-dibromo-2,3-dimethylimidazo[1,2-a]pyridine (1.5 g, 4.93 mmol; Description 1) in dimethylformamide (3 mL) was added, and then the mixture was heated in an Initiator™ Microwave Synthesizer at 100° C. for 1 hour. The cooled mixture was partitioned between ethyl acetate and water. The organic phase was washed with water, then brine, dried (MgSO₄) and evaporated. Purification by chromatography on silica gel (ethyl acetate/toluene), then crystallization from methyl tert-butyl ether gave the title compound. MS (ES+ve): [M+H]⁺ at 7 m/z 359 (C₁₈H₁₉⁷⁹BrN₂O requires [M+H]⁺ at m/z 359).

### Description 3—1-Bromo-2-ethyl-6-methylbenzene

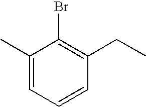

2-Ethyl-6-methylaniline (35 g, 0.259 mol) was added to cooled concentrated hydrobromic acid (120 mL). The stirred suspension was maintained between 5° C. and 9° C. while a solution of sodium nitrite (18.2 g, 0.264 mol) in water (40 mL) was added. A solution of copper (I) bromide (20.4 g, 0.142 mol) in concentrated hydrobromic acid (28 mL) was added and the reaction mixture was allowed to rise to room temperature. The mixture was stirred at room temperature for 1 hour and then 30 minutes at 40° C. After cooling to room temperature hexane was added. The organic layer was washed with water, dried (MgSO₄) and evaporated. Purification by chromatography on silica gel (hexane) gave the title compound. ¹H NMR (CDCl₃) δ 1.22 (t, 3H), 2.42 (s, 3H), 2.78 (q, 2H), 7.04-7.14 (m, 3H).

### Description 4—2-Ethyl-6-methylbenzaldehyde

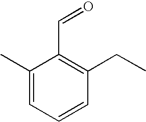

To magnesium (2.65 g, 0.11 mol) in tetrahydrofuran (20 mL) was added a crystal of iodine. The mixture was stirred until the iodine colour had discharged, then ca. 2 mL of 1-bromo-2-ethyl-6-methylbenzene (20 g, 0.10 mol; Description 3) was added. The mixture became warm, a further portion of tetrahydrofuran (60 mL) was added, and then the remaining 1-bromo-2-ethyl-6-methylbenzene in tetrahydrofuran (20 mL) was added over 20 minutes maintaining the temperature between 25° C. and 30° C. The mixture was stirred for a further 1 hour at 40° C. After cooling to 0° C., a solution of dimethylformamide (15.4 mL, 0.20 mol) in tetrahydrofuran (17 mL) was added maintaining the temperature below 9° C. The mixture was stirred at room temperature for 1 hour, then cooled in an ice bath. Saturated ammonium chloride (200 mL) was added, and then the mixture was extracted with diethyl ether. The organic phase was washed with brine, dried (MgSO₄) and evaporated. Purification by chromatography on silica gel (hexane/dichloromethane) gave the title compound. ¹H NMR (CDCl₃) δ 1.25 (t, 3H), 2.60 (s, 3H), 2.98 (q, 2H), 7.09 (d, 1H), 7.12 (d, 1H), 7.35 (t, 1H), 10.61 (s, 1H).

### Description 5—2-Ethyl-6-methylbenzyl alcohol

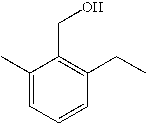

A solution of 2-ethyl-6-methylbenzaldehyde (13.3 g, 89.7 μmol; Description 4) in methanol (120 mL) was cooled in an ice bath. Sodium borohydride (2.92 g, 77.2 mmol) was added portionwise maintaining the temperature below 13° C. The mixture was stirred in the ice bath for 1 hour. The solvent was evaporated under reduced pressure and the residue was partitioned between diethyl ether and water. The organic phase was washed with brine, dried (MgSO₄) and evaporated to give the title compound. ¹H NMR (CDCl₃) δ 1.24 (t, 3H), 2.44 (s, 3H), 2.77 (q, 2H), 4.75 (s, 2H), 7.04-7.18 (m, 3H).

### Description 6—2-Ethyl-6-methylbenzyl bromide

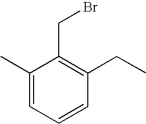

A mixture of 2-ethyl-6-methylbenzyl alcohol (13.25 g, 88.3 mmol; Description 5) and concentrated hydrobromic acid (150 mL) was stirred at room temperature for 2 hours, then cooled in an ice bath. Water (350 mL) was added, and then the mixture was extracted twice with diethyl ether. The combined organic phases were washed with brine, dried (MgSO₄) and evaporated to give the title compound. ¹H NMR (CDCl₃) δ 1.29 (t, 3H), 2.43 (s, 3H), 2.77 (q, 2H), 4.60 (s, 2H), 7.03-7.18 (m, 3H).

### Description 7—Methyl 8-{[(2-ethyl-6-methylphenyl)methyl]amino}-2,3-dimethylimidazo[1,2-a]pyridine-6-carboxylate

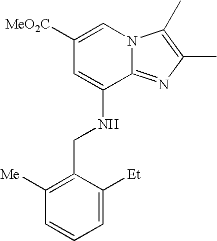

To a solution of methyl 8-amino-2,3-dimethylimidazo[1,2-a]pyridine-6-carboxylate hydrobromide (0.50 g, 1.67 mmol) (ref: WO02/20523) in dimethylformamide (30 mL) was added sodium carbonate (0.565 g, 5.33 mmol) and 2-ethyl-6-methylbenzyl bromide (0.355 g, 1.67 mmol; Description 6). The mixture was stirred at room temperature for 20 hours, and then partitioned between ethyl acetate and water. The organic phase was washed with water, dried (MgSO₄) and evaporated to give the title compound. MS (ES+ve): [M+H]⁺ at m/z 352 (C₂₁H₂₅N₃O₂ requires [M+H]⁺ at m/z 352).

### Description 8—Sodium 8-{[(2-Ethyl-6-methylphenyl)methyl]amino}-2,3-dimethylimidazo[1,2-a]pyridine-6-carboxylate

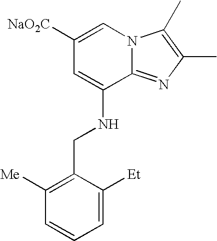

A mixture of methyl 8-{[(2-ethyl-6-methylphenyl)methyl]amino}-2,3-dimethylimidazo[1,2-a]pyridine-6-carboxylate (0.546 g, 1.56 mmol; Description 7), 2M aqueous sodium hydroxide (8 mL) and dioxane (8 mL) was refluxed for 30 minutes. The mixture was concentrated under reduced pressure, and then the residue was acidified by addition of 2M aqueous hydrochloric acid. The mixture was then basified by addition of saturated aqueous sodium bicarbonate and the resulting solid was collected by filtration to give the title compound. MS (ES+ve): (free acid observed) [M+H]⁺ at m/z 338 (C₂₀H₂₃N₃O₂ requires [M+H]⁺ at m/z 338).

### Description 9—8-{[(2-Ethyl-6-methylphenyl)methyl]amino}-2,3-dimethylimidazo[1,2-a]pyridine-6-carbohydrazide

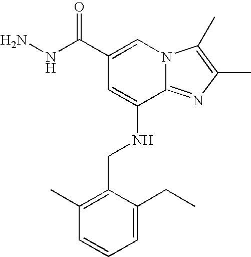

To a suspension of sodium 8-{[(2-ethyl-6-methylphenyl)methyl]amino}-2,3-dimethylimidazo[1,2-a]pyridine-6-carboxylate (406 mg, 1.13 mmol; Description 8) in tetrahydrofuran (8 mL) and dimethylformamide (4 mL) was added N,N′-carbonyldiimidazole (351 mg, 2.17 mmol). After 1.5 hours at 60° C., the solution was cooled to 40° C. and hydrazine hydrate (0.177 mL, 3.64 mmol) was added. After 20 hours a further portion of hydrazine hydrate (0.177 mL, 3.64 mmol) was added, then after a further 4 hours N,N′-carbonyldiimidazole (351 mg, 2.17 mmol) was added, and then after a further 30 minutes hydrazine hydrate (0.177 mL, 3.64 mmol) was added and the mixture stirred for 20 hours. The reaction mixture was applied to an Isolute® PE-AX cartridge. Elution with methanol gave the crude product. Elution with 10% acetic acid in methanol allowed the recovery of 8-{[(2-Ethyl-6-methylphenyl)methyl]amino}-2,3-dimethylimidazo[1,2-a]pyridine-6-carbohydrazide. The crude product was further purified by chromatography on silica gel (dichloromethane/methanol) to give the title compound. MS (ES+ve): [M+H]⁺ at m/z 352 (C₂₀H₂₅N₅O requires [M+H]⁺ at m/z 352).

### Description 10—N-[(Dimethylamino)methylidene]-8-{[(2-ethyl-6-methylphenyl)methyl]amino}-2,3-dimethylimidazo[1,2-a]pyridine-6-carboxamide

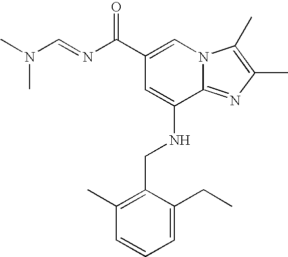

A mixture of 8-{[(2-ethyl-6-methylphenyl)methyl]amino}-2,3-dimethylimidazo[1,2-a]pyridine-6-carboxamide (54 mg, 0.167 mmol; WO99/55706) and dimethylformamide dimethyl acetal (3 mL) was heated under reflux for 7 hours. After cooling, the mixture was evaporated to dryness. The residue was purified by chromatography on silica gel (ethyl acetate) to give the title compound. MS (ES+ve): [M+H]⁺ at m/z 392 (C₂₃H₂₉N₅O requires [M+H]⁺ at m/z 392).

### Description 11—8-{[(2-Ethyl-6-methylphenyl)methyl]amino}-2,3-dimethylimidazo[1,2-a]pyridine-6-carbonitrile

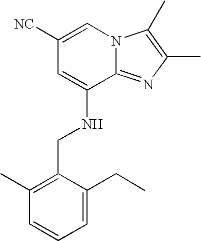

A solution of 8-{[(2-ethyl-6-methylphenyl)methyl]amino}-2,3-dimethylimidazo[1,2-a]pyridine-6-carboxamide (1.034 mg, 3.07 mmol; WO99/55706) in tetrahydrofuran (20 mL) was cooled in an ice bath. Triethylamine (2.7 mL, 36.7 mmol) was added, and then a solution of trifluoroacetic anhydride (1.65 mL, 11.9 mmol) in tetrahydrofuran (10 mL) was added dropwise over 5 minutes. The mixture was stirred for 30 minutes at 0° C. Water (30 mL) was added, and then the mixture was concentrated under reduced pressure. The concentrate was extracted with ethyl acetate, and the extract washed with water, then brine, dried (MgSO₄) and evaporated. The residue was dissolved in methanol (50 mL), water (10 mL) and potassium carbonate (1.65 g) were added, and the mixture stirred at room temperature for 45 minutes. After concentration the mixture was partitioned between ethyl acetate and water. The organic phase was washed with brine, dried (MgSO₄) and evaporated. Purification by chromatography on silica gel (ethyl acetate/toluene) gave the title compound. MS (ES+ve): [M+H]⁺ at m/z 319 (C₂₀H₂₂N₄ requires [M+H]⁺ at m/z 319).

### Description 12. Methyl 5-(8-{[(2,6-dimethylphenyl)methyl]amino}-2,3-dimethylimidazo[1,2-a]pyridin-6-yl)-2-furancarboxylate

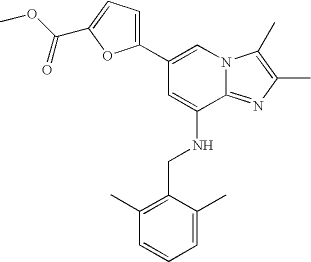

To a solution of 6-bromo-N-[(2,6-dimethylphenyl)methyl]-2,3-dimethylimidazo[1,2-a]pyridin-8-amine (190 mg, 0.53 mmol; WO98/37080) in dioxane (3 mL) was added diboron pinacol ester (135 mg, 0.53 mmol), 1,1′-bis(diphenylphosphino)ferrocene (15 mg, 0.027 mmol) and potassium acetate (78 mg, 0.80 mmol). After degassing the reaction mixture with argon, [1,1′-bis(diphenylphosphino)ferrocene]dichloropalladium (22 mg, 0.027 mmol) was added and the mixture heated in an Initiator™ Microwave Synthesizer at 120° C. for 2 hours. To the cooled reaction mixture containing N-[(2,6-dimethylphenyl)methyl]-2,3-dimethyl-6-(4,4,5,5-tetramethyl-1,3,2-dioxaborolan-2-yl)imidazo[1,2-a]pyridin-8-amine was added methyl 5-bromo-2-furancarboxylate (109 mg, 0.53 mmol) and cesium carbonate (259 mg, 0.80 mmol). After degassing the reaction mixture with argon, tetrakis(triphenylphosphine)palladium(0) (31 mg, 0.027 mmol) was added and the mixture heated in an Initiator™ Microwave Synthesizer at 110° C. for 5.5 hours. The cooled mixture was partitioned between ethyl acetate and water. The organic phase was washed with water, then brine, dried (MgSO₄) and evaporated. The residue was purified by chromatography on silica gel (ethyl acetate/hexane) to give the title compound. MS (ES+ve): [M+H]⁺ at m/z 404 (C₂₄H₂₅N₃O₃ requires [M+H]⁺ at m/z 404).

### Description 13. 5-(8-{[(2,6-Dimethylphenyl)methyl]amino}-2,3-dimethylimidazo[1,2-a]pyridin-6-yl)-2-furancarboxylic acid

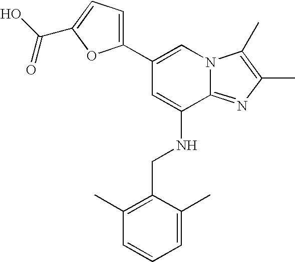

To a solution of methyl 5-(8-{[(2,6-dimethylphenyl)methyl]amino}-2,3-dimethylimidazo[1,2-a]pyridin-6-yl)-2-furancarboxylate (85 mg, 0.21 mmol; Description 12) in tetrahydrofuran (5 mL) was added an aqueous solution of lithium hydroxide (0.5N, 5 mL). The mixture was stirred at room temperature for 16 hours. The mixture was acidified by the addition of 5N HCl, and then partitioned between ethyl acetate and water. The organic phase was evaporated to give the title compound. MS (ES+ve): [M+H]⁺ at m/z 390 (C₂₃H₂₃N₃O₃ requires [M+H]⁺ at m/z 390).

### Description 14. 1-Hydroxybenzotriazole ammonium salt

Ammonium hydroxide (6.2 mL) was added dropwise to a solution of 1-hydroxybenzotriazole hydrate (10 g, 65 mmol) in tetrahydrofuran (100 mL). A further portion of tetrahydrofuran (100 mL) was added and the resulting precipitate was collected by filtration, washed with tetrahydrofuran, and dried under vacuum to give the title compound. ¹H NMR (DMSO-d₆) δ 7.09 (m+br s, 6H), 7.42 (m, 1H), 7.63 (m, 1H).

### Description 15. Methyl 6-(8-{[(2,6-dimethylphenyl)methyl]amino}-2,3-dimethylimidazo[1,2-a]pyridin-6-yl)-2-pyridinecarboxylate

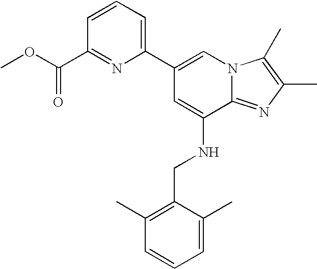

To a solution of 6-bromo-N-[(2,6-dimethylphenyl)methyl]-2,3-dimethylimidazo[1,2-a]pyridin-8-amine (105 mg, 0.293 mmol; WO98/37080) in dioxane (3.5 mL) was added diboron pinacol ester (75 mg, 0.29 mmol), 1,1′-bis(diphenylphosphino)ferrocene (8 mg, 0.015 mmol) and potassium acetate (43 mg, 0.44 mmol). After degassing the reaction mixture with argon, [1,1′-bis(diphenylphosphino)ferrocene]dichloropalladium (12 mg, 0.015 mmol) was added and the mixture heated in an Initiator™ Microwave Synthesizer at 120° C. for 2 hours.

To the cooled reaction mixture containing N-[(2,6-dimethylphenyl)methyl]-2,3-dimethyl-6-(4,4,5,5-tetramethyl-1,3,2-dioxaborolan-2-yl)imidazo[1,2-a]pyridin-8-amine was added methyl 6-bromo-2-pyridinecarboxylate (76 mg, 0.35 mmol) and cesium carbonate (143 mg, 0.44 mmol). After degassing the reaction mixture with argon, tetrakis(triphenylphosphine)palladium(0) (17 mg, 0.015 mmol) was added and the mixture heated in an Initiator™ Microwave Synthesizer at 110° C for 8 hours. The cooled mixture was partitioned between ethyl acetate and water. The organic phase was washed with water, then brine, dried (MgSO₄) and evaporated. The residue was purified by chromatography on silica gel (ethyl acetate/hexane) to give the title compound. MS (ES+ve): [M+H]⁺ at m/z 415 (C₂₅H₂₆N₄O₂ requires [M+H]⁺ at m/z 415).

### Description 16. 1H-Pyrazole-4-carboxamide

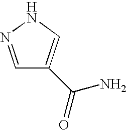

A solution of 1H-pyrazole-4-carboxylic acid (56 mg, 0.50 mmol) in thionyl chloride (4 mL) was heated at 75° C. for 4 hours. After cooling, the solution was evaporated to dryness. The residue was dissolved in dioxane (5 mL) and this solution was added to a rapidly stirred solution of ammonium hydroxide in water. After 1 hour the mixture was evaporated to dryness to give the title compound.

### Description 17—N-[(2,6-Dimethylphenyl)methyl]-6-ethynyl-2,3-dimethylimidazo[1,2-a]pyridin-8-amine

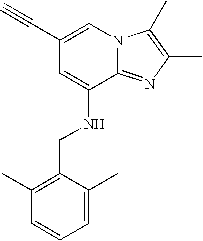

To a solution of 6-bromo-N-[(2,6-dimethylphenyl)methyl]-2,3-dimethylimidazo[1,2-a]pyridin-8-amine (1.0 g, 2.8 mmol; WO98/37080) in triethylamine (10 mL) was added (trimethylsilyl)acetylene (0.47 mL, 3.3 mmol) and copper(I) iodide (23 mg, 0.12 mmol). After degassing the reaction mixture with argon, bis(triphenylphosphine)palladium(II) chloride (50 mg, 0.07 mmol) was added and the reaction stirred at 45° C. under argon for 16 hours. The solvent was evaporated and then diluted with ethyl acetate and dilute aqueous ammonium chloride solution. After separation of the layers, the aqueous phase was re-extracted with ethyl acetate and then the combined organic layers dried (MgSO₄) and evaporated. The crude product was dissolved in methanol (50 mL) and treated with 1M aqueous potassium hydroxide solution. The reaction mixture was stirred for 1 hour at room temperature and the solvent was evaporated. The residue was partitioned between ethyl acetate and water. After separation of the layers, the aqueous phase was re-extracted with ethyl acetate. The combined organic layers were dried (MgSO₄) and evaporated. Purification by chromatography on silica gel (ethyl acetate/hexane) gave the title compound. MS (ES+ve): [M+H]⁺ at m/z 304 (C₂₀H₂₁N₃ requires [M+H]⁺ at m/z 304).

### Description 18—N-[(2,6-dimethylphenyl)methyl]-2,3-dimethyl-6-(5-nitro-2-pyridinyl)imidazo[1,2-a]pyridin-8-amine

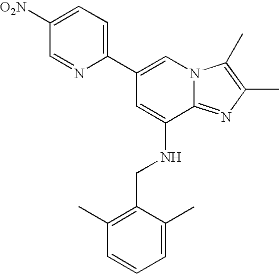

To a solution of 6-bromo-N-[(2,6-dimethylphenyl)methyl]-2,3-dimethylimidazo[1,2-a]pyridin-8-amine (100 mg, 0.28 mmol; WO98/37080) in dioxane (3 mL) was added diboron pinacol ester (85 mg, 0.34 mmol), 1,1′-bis(diphenylphosphino)ferrocene (8 mg, 0.014 mmol) and potassium acetate (82 mg, 0.84 mmol). After degassing the reaction mixture with argon, [1,1′-bis(diphenylphosphino)ferrocene]dichloropalladium (11 mg, 0.014 mmol) was added and the mixture heated to reflux for 4 hours. To the cooled reaction mixture containing N-[(2,6-dimethylphenyl)methyl]-2,3-dimethyl-6-(4,4,5,5-tetramethyl-1,3,2-dioxaborolan-2-yl)imidazo[1,2-a]pyridin-8-amine was added 2-bromo-5-nitropyridine (57 mg, 0.28 mmol) and cesium carbonate (137 mg, 0.42 mmol). After degassing the reaction mixture with argon, tetrakis(triphenylphosphine)palladium(0) (16 mg, 0.014 mmol) was added and the mixture heated at 80° C. for 16 hours. The cooled mixture was loaded onto an Isolute® SCX cartridge, eluting with methanol, then 2M NH₃ in methanol. Fractions containing the product were purified by silica gel chromatography (ethyl acetate/hexane) to give the title compound. MS (ES+ve): [M+H]⁺ at m/z 402 (C₂₃H₂₃N₅O₂ requires [M+H]⁺ at m/z 402).

### Description 19—1-(8-Amino-2,3-dimethylimidazo[1,2-a]pyridin-6-yl)-2(1H)-pyridinone

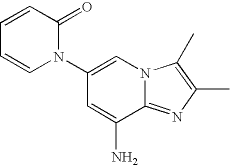

A mixture containing 6,8-dibromo-2,3-dimethylimidazo[1,2-a]pyridine (Description 1, 6 g, 25 mmol), 2-hydroxypyridine (4.75 g, 50 mmol), potassium carbonate (6.9 g, 50 mmol), copper(I) iodide (0.95 g, 5 mmol) and N-methylpyrrolidine (50 mL) was stirred at 195° C. for 8 hours. The mixture was filtered through celite, washing with ethyl acetate and water, and then loaded onto an Isolute® SCX cartridge, washing with methanol and then eluted with 2M NH₃/MeOH. Solvent was removed in vacuo from appropriate fractions and the resulting mixture was purified on silica eluting with a 0-20% methanol in ethyl acetate gradient to give, upon evaporation, the title compound. MS (ES+ve): [M+H]⁺ at m/z 255 (C₁₄H₁₄N₄O requires [M+H]⁺ at m/z 255).

### Description 20: 2,3-Dimethyl-6-(1H-pyrazol-1-yl)imidazo[1,2-a]pyridin-8-amine

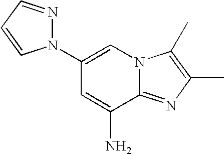

A mixture containing 6,8-dibromo-2,3-dimethylimidazo[1,2-a]pyridine (Description 1, 500 mg, 2.08 mmol), pyrazole (283 mg, 4.16 mmol), potassium carbonate (6574 mg, 4.16 mmol), copper(I) iodide (80 mg, 0.41 mmol) and N-methylpyrrolidine (5 mL) was stirred under microwave heating at 195° C. for 6 hours. Methanol (5 mL) was added and the resulting mixture loaded onto an Isolute® SCX cartridge, washed with methanol and then eluted with 2M NH₃/MeOH. Solvent was removed from appropriate fractions in vacuo. The resulting mixture was purified on silica eluting with a 0-10% methanol in ethyl acetate gradient to give, upon evaporation an impure solid, which was triturated with hexane to give the title compound. MS (ES+ve): [M+H]⁺ at m/z 228 (C₁₂H₁₃N₅ requires [M+H]⁺ at m/z 228).

### Description 21—6-Bromo-2,3-dimethyl-8-({[4-(methyloxy)phenyl]methyl}oxy)imidazo[1,2-a]pyridine

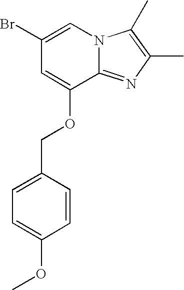

Sodium hydride (80 mg of a 60% dispersion in mineral oil; 2.0 mmol) was added to a stirred solution of [4-(methyloxy)phenyl]methanol (250 μl; 2.0 mmol) in dry dimethylformamide (3 mL) and the mixture stirred under argon for 90 minutes. 6,8-Dibromo-2,3-dimethylimidazo[1,2-a]pyridine (Description 1, 300 mg, 1.0 mmol) was added and the resulting mixture was heated at 90° C. under argon for 18 hours.

The mixture was applied to an Isolute® SCX cartridge and eluted with methanol followed by 2M NH₃ in methanol. The basic fractions were combined and evaporated under reduced pressure. The residue was purified by column chromatography on silica eluting with 2-1 n-pentane-ethyl acetate and then ethyl acetate to afford the title compound. ¹H NMR (CDCl₃) δ 2.35 (3H, s), 2.40 (3H, s), 3.81 (3H, s), 5.22 (2H, s), 6.54 (H, s), 6.90 (2H, m), 7.41 (2H, m), 7.57 (H, s)

### Description 22—1-[2,3-Dimethyl-8-({[4-(methyloxy)phenyl]methyl}oxy)imidazo[1,2-a]pyridin-6-yl]-2(1H)-pyridinone

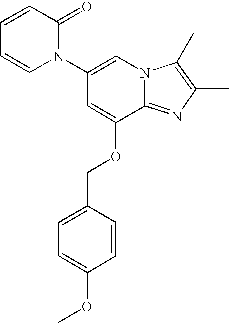

A mixture of 6-bromo-2,3-dimethyl-8-({[4-(methyloxy)phenyl]methyl}oxy)imidazo[1,2-a]pyridine (90 mg, 0.25 mmol. Description 21), 2-hydroxypyridine (50 mg, 0.50 mmol) and copper(I) iodide (10 mg, 0.05 mmol) in dry dimethylformamide (3 mL) was heated in an Initiator™ Microwave Synthesizer at 150° C. for 4 hours. The mixture was filtered through Celite and the pad washed with ethyl acetate. The filtrate was evaporated under reduced pressure and the residue applied to an Isolute® SCX cartridge and eluted with methanol followed by 2M NH₃ in methanol. The basic fractions were combined and evaporated under reduced pressure. This residue was purified by column chromatography on silica eluting with ethyl acetate and then 3-97 2M NH₃ in methanol-dichloromethane to afford the title compound MS (ES+ve): [M+H]⁺ at m/z 376 (C₂₂H₂₁N₃O₃ requires [M+H]⁺ at m/z 376).

### Description 23—1-(8-Hydroxy-2,3-dimethylimidazo[1,2-a]pyridin-6-yl)-2(1H)-pyridinone

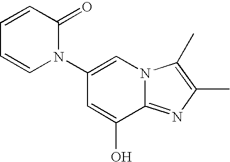

Trifluoroacetic acid (5 mL) was added to solution of 1-[2,3-dimethyl-8-({[4-(methyloxy)phenyl]methyl}oxy)imidazo[1,2-a]pyridin-6-yl]-2(1H)-pyridinone (447 mg, 1.19 mmol Description 22) in dichloromethane (5 mL) and the mixture stirred at room temperature for 3 hours. The mixture was purified on an Isolute® SCX cartridge and eluted with methanol followed by 2M NH₃ in methanol. The basic fractions were combined and evaporated under reduced pressure. This residue was triturated with diethyl ether and the title compound obtained by filtration MS (ES+ve): [M+H]⁺ at m/z 256 (C₁₄H₁₃N₃O₂ requires [M+H]⁺ at m/z 256).

### Description 24. 6-bromo-2-methylimidazo[1,2-a]pyridin-8-amine

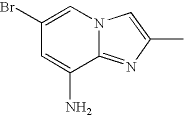

A mixture of 2,3-diamino-5-bromopyridine (10 g, 53 mmol), 1-chloro-2-propanone (5.41 g, 59 mmol), and ethanol (50 ml) was heated to reflux for 16 hours. The cooled reaction mixture was treated with water (100 ml) and the solid collected by filtration to give the title compound. MS (ES+ve): [M+H]⁺ at m/z 226/228 (C₈H₈BrN₃ requires [M+H]⁺ at m/z 226/228).

### Description 25. 6-bromo-N-[(2,6-dimethylphenyl)methyl]-2-methylimidazo[1,2-a]pyridin-8-amine

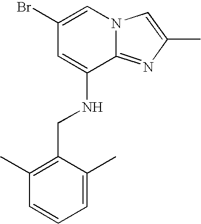

A mixture of 6-bromo-2-methylimidazo[1,2-a]pyridin-8-amine (Description 24, 5.50 g, 25 mmol), 2,6-dimethylbenzyl chloride (3.78 g, 25 mmol), potassium iodide (1.63 g, 10 mmol), sodium carbonate (9.35 g, 88 mmol) and dimethylformamide (10 ml) were stirred at room temperature for 16 hours. The solvent was evaporated and the mixture taken up in ethyl acetate/water. The mixture was filtered and the organic layer separated, washed with brine, dried (MgSO₄), and the solvent evaporated. The residue was purified by silica gel chromatography (ethyl acetate/hexane) to give the title compound. MS (ES+ve): [M+H]⁺ at m/z 344/346 (C₁₇H₁₈BrN₃ requires [M+H]⁺ at m/z 344/346).

### Description 26. 1-(8-{[(2,6-Dimethylphenyl)methyl]amino}-2-methylimidazo[1,2-a]pyridin-6-yl)-2(1H)-pyridinone

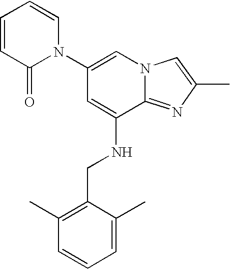

A mixture of 6-bromo-N-[(2,6-dimethylphenyl)methyl]-2-methylimidazo[1,2-a]pyridin-8-amine (Description 25, 2.54 g, 7.4 mmol), 2-hydroxypyridine (844 mg, 8.9 mmol), copper (I) iodide (281 mg, 1.5 mmol), potassium carbonate (2.04 g, 14 mmol), and N-methyl-2-pyrrolidinone (10 ml) was heated in an Initiator™ Microwave Synthesizer at 155° C. for 30 min with cooling on. The cooled reaction mixture was loaded onto an Isolute® SCX cartridge, washed with methanol and then eluted with 1M NH₃/MeOH. The solvent was evaporated and the residue purified by silica gel chromatography (ethyl acetate/hexane) to give the title compound. MS (ES+ve): [M+H]⁺ at m/z 359 (C₂₂H₂₂N₄O requires [M+H]⁺ at m/z 359).

### Description 27. 1-(3-[(Dimethylamino)methyl]-8-{[(2,6-dimethylphenyl)methyl]amino}-2-methylimidazo[1,2-a]pyridin-6-yl)-2(1H)-pyridinone

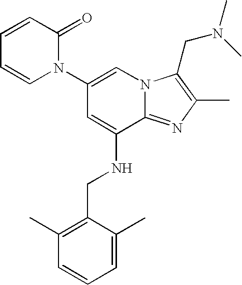

A mixture of 1-(8-{[(2,6-dimethylphenyl)methyl]amino}-2-methylimidazo[1,2-a]pyridin-6-yl)-2(1H)-pyridinone (Description 26, 593 mg, 1.65 mmol), paraformaldehyde (55 mg, 1.81 mmol) and dimethylamine hydrochloride (148 mg, 1.81 mmol) in methanol (30 ml) was heated at reflux for 2 hours. The mixture was allowed to cool and was was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane to afford the title compound MS (ES+ve): [M+H]⁺ at m/z 416 (C₂₅H₂₉N₅O requires [M+H]⁺ at m/z 416).

### Description 28. [8-{[(2,6-Dimethylphenyl)methyl]amino}-2-methyl-6-(2-oxo-1(2H)-pyridinyl)imidazo[1,2-a]pyridin-3-yl]-N,N,N-trimethylmethanaminium iodide

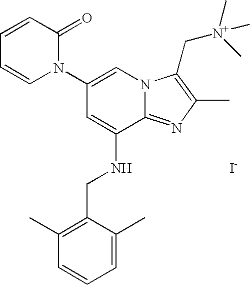

A mixture of 1-(3-[(dimethylamino)methyl]-8-{[(2,6-dimethylphenyl)methyl]amino}-2-methylimidazo[1,2-a]pyridin-6-yl)-2(1H)-pyridinone (Description 27, 423 mg, 1.02 mmol) and iodomethane (70 μl, 1.12 mmol) in ethanol (8 ml) was stirred a room temperature for 18 hours. The resulting solid was collected by filtration and dried in a vacuum oven to afford the title compound. Proton NMR (d6-DMSO) δ 8.34 (H, s), 7.74 (H, m), 7.58 (H, m), 7.17-7.07 (3H, m), 6.53 (H, m), 6.48 (H, s), 6.38 (H, m), 5.45 (H, t), 4.88 (2H, s), 4.35 (2H, d), 3.06 (9H, s), 2.42 (3H, s), 2.34 (6H, s).

### Description 29. N-[(2,6-dimethylphenyl)methyl]-2-methyl-6-(1H-1,2,4-triazol-1-yl)imidazo[1,2-a]pyridin-8-amine

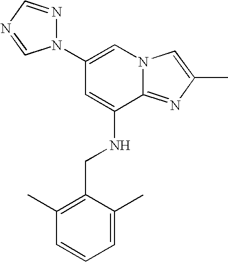

A mixture of 6-bromo-N-[(2,6-dimethylphenyl)methyl]-2-methylimidazo[1,2-a]pyridin-8-amine (Description 25; 1 g, 2.91 mmol), 1,2,4-triazole (0.602 mg, 8.72 mmol), copper(I) iodide (170 mg, 0.891 mmol), cesium carbonate (1.99 g, 6.1 mmol) and N,N′-dimethylethylenediamine (85 mg, 0.891 mmol) in N-methyl-2-pyrrolidone (5 mL) was heated in an Initiator™ Microwave Synthesizer at 180° C. for 16 hours. The cooled mixture was partitioned between ethyl acetate (3×20 ml) and water (50 ml). The combined organic extracts were washed with brine (3×50 ml) and dried (Na₂SO₄). The solvent was evaporated and the residue purified by chromatography on silica. Elution with hexane/ethyl acetate (25% and increasing the polarity to 75%) gave the title compound as a pale brown solid. MS (ES+ve): [M+H]⁺ at m/z 333 (C₁₉H₂₀N₆ requires [M+H]⁺ at m/z 333).

### Description 30. 3-[(dimethylamino)methyl]-N-[(2,6-dimethylphenyl)methyl]-2-methyl-6-(1H-1,2,4-triazol-1-yl)imidazo[1,2-a]pyridin-8-amine

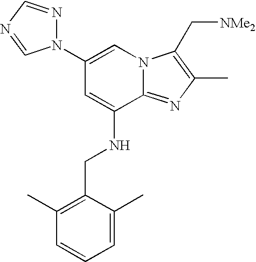

A mixture of N-[(2,6-dimethylphenyl)methyl]-2-methyl-6-(1H-1,2,4-triazol-1-yl)imidazo[1,2-a]pyridin-8-amine (Description 29; 0.4 g, 2.91 mmol), paraformaldehyde (43 mg, 1.44 mmol) and dimethylamine hydrochloride (118 mg, 1.44 mmol) in dry methanol (5 mL) and was heated under reflux for 3 hours. The cooled mixture was evaporated and the residue purified by chromatography on silica. Elution with dichloromethane/methanolic ammonia (0% and increasing the polarity to 10%) gave the title compound as a colourless foam. MS (ES+ve): [M+H]⁺ at m/z 390 (C₂₂H₂₇N₇ requires [M+H]⁺ at m/z 390).

### Description 31. 1-(8-{[(2,6-dimethylphenyl)methyl]amino}-2-methylimidazo[1,2-a]pyridin-6-yl)-4-methyl-2(1H)-pyridinone

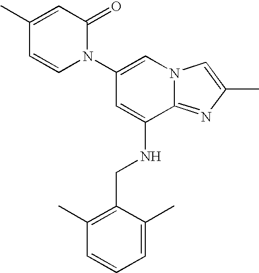

A mixture of 6-bromo-N-[(2,6-dimethylphenyl)methyl]-2-methylimidazo[1,2-a]pyridin-8-amine (Description 25, 500 mg, 1.45 mmol), copper iodide (127 mg, 0.44 mmol), N,N′-dimethyl-1,2-ethanediamine (0.046 ml, 0.44 mmol) and 2-hydroxy-4-methylpyridine (317 mg, 2.90 mmol) in NMP (5 ml) was heated in an Initiator™ Microwave Synthesizer at 180° C. for 16 hours. The reaction mixture was partitioned between ethyl acetate and water, the organic phase washed with water, brine, dried (MgSO₄) and evaporated. The residue was purified by chromatography on silica gel (ethyl acetate/hexane) and evaporated to give the title compound. MS (ES+ve): [M+H]⁺ at m/z 373 (C₂₃H₂₄N₄O requires [M+H]⁺ at m/z 373).

### Description 32. 1-(3-[(dimethylamino)methyl]-8-{[(2,6-dimethylphenyl)methyl]amino}-2-methylimidazo[1,2-a]pyridin-6-yl)-4-methyl-2(1H)-pyridinone

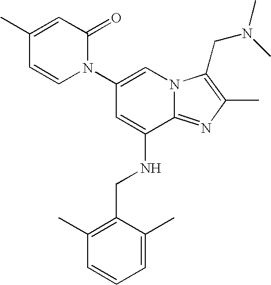

A mixture of 1-(8-{[(2,6-dimethylphenyl)methyl]amino}-2-methylimidazo[1,2-a]pyridin-6-yl)-4-methyl-2(1H)-pyridinone (Description 31, 226 mg, 0.607 mmol), paraformaldehyde (20 mg, 0.67 mmol) and dimethylamine hydrochloride (50 mg, 0.67 mmol) was refluxed in methanol for 6 hours, allowed to cool overnight, more paraformaldehyde (20 mg, 0.67 mmol) and dimethylamine hydrochloride (50 mg, 0.67 mmol) added and the reaction refluxed for 3 hours. The reaction mixture was allowed to cool, loaded onto an Isolute® SCX cartridge, eluted with methanol, then 1M NH₃ in methanol which gave, after evaporation, the product which was further purified by chromatography on silica gel (2M methanolic ammonia/dichloromethane) and evaporated to give the title compound. MS (ES+ve): [M+H]⁺ at m/z 430 (C₂₆H₃₁N₅O requires [M+H]⁺ at m/z 430).

### Description 33. [8-{[(2,6-dimethylphenyl)methyl]amino}-2-methyl-6-(4-methyl-2-oxo-1(2H)-pyridinyl)imidazo[1,2-a]pyridin-3-yl]-N,N,N-trimethylmethanaminium iodide

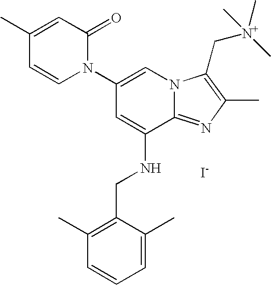

A mixture of 1-(3-[(dimethylamino)methyl]-8-{[(2,6-dimethylphenyl)methyl]amino}-2-methylimidazo[1,2-a]pyridin-6-yl)-4-methyl-2(1H)-pyridinone (Description 32, 193 mg, 0.45 mmol) and methyl iodide (0.031 ml, 0.50 mmol) were stirred in ethanol (2 ml) for 6 hours and evaporated. The residue was triturated with diethyl ether and dried under reduced pressure to give the title compound. ¹H NMR (DMSO) δ 2.22 (s, 3H), 2.34 (s, 6H), 2.41 (s, 3H), 3.05 (s, 9H), 3.35 (d, 2H), 4.87 (s, 2H), 5.44 (t, 1H), 6.25 (dd, 1H), 6.34 (s, 1H), 6.45 (d, 1H), 7.07-7.17 (m, 3H), 7.62 (d, 1H), 8.30 (d, 1H)

### Description 34: 6,8-dibromo-2-methylimidazo[1,2-a]pyridine

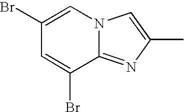

A mixture of 3,5-dibromo-2-pyridinamine (50.4 g, 0.2 mol) and 1-chloro-2-propanone (23.7 ml, 0.3 mol) in N-methyl-2-pyrrolidone (140 ml) was stirred in an Initiator™ Microwave Synthesizer at 160° C. for 1 h. The precipitate formed was filtered off, washed with ether then dried under vacuum at 40° C. for 1 h. The residue was dissolved in water and the aqueous phase was basified with a 2N aqueous sodium hydroxide solution. The precipitate formed was filtered off and washed with ethyl acetate then dissolved in dichloromethane, dried (MgSO₄) and concentrated in vacuo to give the title compound (31 g, 54%) as a brown solid which was used in the next step without further purification. MS (ES+ve): [M+H]⁺ at m/z 289 (C₈H₆⁷⁹Br₂N₂ requires [M+H]⁺at m/z 289).

### Description 35: 6-Bromo-2-methyl-8-({[4-(methyloxy)phenyl]methyl}oxy)imidazo[1,2-a]pyridine

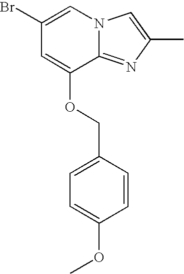

To a suspension of sodium hydride (60% dispersion in mineral oil, 4.0 g, 0.1 mol) at 0° C. in dimethylformamide (300 ml) was slowly added [4-(methyloxy)phenyl]methanol (12.4 ml, 0.1 mol) via syringe and the resulting mixture was allowed to warm to room temperature over 1 hour. 6,8-Dibromo-2-methylimidazo[1,2-a]pyridine (Description 34, 14.45 g, 0.05 mol) was added and the resulting mixture was stirred at 60° C. for 4 hours then cooled to room temperature and concentrated in vacuo. The residue was partitioned between ethyl acetate and a saturated aqueous sodium bicarbonate solution and the layers were separated. The aqueous phase was extracted twice with ethyl acetate and the combined organic phases were washed with a saturated aqueous sodium bicarbonate solution, dried (MgSO₄) and concentrated in vacuo. Purification of the residue by flash chromatography on silica gel (hexane/ethylacetate: 4/1 to 1/1) gave the title compound (10.1 g, 58%) as a brown solid. MS (ES+ve): [M+H]⁺ at m/z 347 (C₁₆H₁₅⁷⁹BrN₂O₂ requires [M+H]⁺ at m/z 347).

### Description 36: 1-[2-Methyl-8-({[4-(methyloxy)phenyl]methyl}oxy)imidazo[1,2-a]pyridin-6-yl]-2(1H)-pyridinone

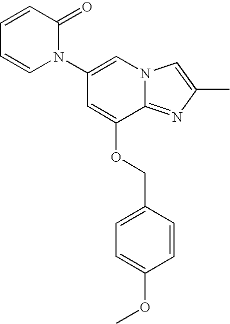

To 6-bromo-2-methyl-8-({[4-(methyloxy)phenyl]methyl}oxy)imidazo[1,2-a]pyridine (Description 35, 4.2 g, 12.1 mmol), copper(I) iodide (920 mg, 4.8 mmol), 2-hydroxypyridine (2.3 g, 24.2 mmol) and potassium phosphate (5.1 g, 24.2 mmol) in 1,4-dioxan (100 ml) was added N,N′-dimethyl-1,2-ethanediamine (1 ml, 9.7 mmol) and the resulting mixture was stirred at reflux for 6 h. Copper(I) iodide (920 mg, 4.8 mmol) and N,N′-dimethyl-1,2-ethanediamine (1 ml, 9.7 mmol) were added and the resulting mixture was stirred at reflux for another 16 h then cooled to room temperature and partitioned between water and ethyl acetate. The insoluble residues were filtered off and most of the solvents were removed in vacuo. The solid residue was washed with water and ethyl acetate then dissolved in dichloromethane, dried (MgSO₄) and concentrated in vacuo to give the title compound (2.82 g, 64%). MS (ES+ve): [M+H]⁺ at m/z 362 (C₂₁H₁₉N₃O₃ requires [M+H]⁺ at m/z 362).

### Description 37: 1-(8-hydroxy-2-methylimidazo[1,2-a]pyridin-6-yl)-2(1H)-pyridinone trifluoroacetic acid salt

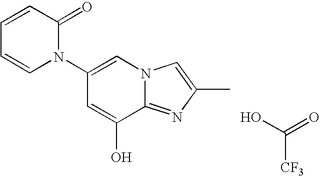

To a suspension of 1-[2-methyl-8-({[4-(methyloxy)phenyl]methyl}oxy)imidazo[1,2-a]pyridin-6-yl]-2(1H)-pyridinone (Description 36, 2.46 g, 6.8 mmol) in dichloromethane (20 ml) at room temperature was added trifluoroacetic acid (20 ml) dropwise and the resulting solution was stirred for 30 min then concentrated in vacuo. The residue was co-evaporated two times with dichloromethane then triturated with methanol. The insoluble material was filtered off and the mother liquors were removed in vacuo to give the title compound (1.91 g, 79%) as a white solid. MS (ES+ve): [M+H]⁺ at m/z 242 (C₁₂H₁₁N₃O₂ requires [M+H]⁺ at m/z 242).

### Description 38: 1-(8-{[(2,6-dimethylphenyl)methyl]oxy}-2-methylimidazo[1,2-a]pyridin-6-yl)-2(1H)-pyridinone

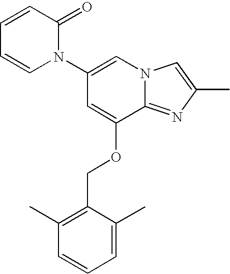

To a suspension of 1-(8-hydroxy-2-methylimidazo[1,2-a]pyridin-6-yl)-2(1H)-pyridinone trifluoroacetic acid salt (Description 37, 1.9 g, 5.4 mmol) and potassium carbonate (3 g, 21.6 mmol) in dimethylformamide (35 ml) was added 2-(bromomethyl)-1,3-dimethylbenzene (1.3 g, 6.5 mmol) and the resulting suspension was stirred at room temperature for 3 hours. Potassium carbonate (0.6 g, 4.3 mmol) and 2-(bromomethyl)-1,3-dimethylbenzene (0.26 g, 1.3 mmol) were then added and the solution stirred for another 30 minutes. The mixture was partitioned between water and ethyl acetate and the two phases were separated. The aqueous phase was extracted with ethyl acetate and the combined organic phases were washed three times with a saturated aqueous sodium bicarbonate, dried (MgSO₄) and concentrated in vacuo. The residue was triturated with hexane to give the title compound (1.68 g, 86%) as a white solid. MS (ES+ve): [M+H]⁺ at m/z 360 (C₂₂H₂₁N₃O₂ requires [M+H]⁺ at m/z 360).

### Description 39: 1-[3-bromo-2-methyl-8-({[4-(methyloxy)phenyl]methyl}oxy)imidazo[1,2-a]pyridin-6-yl]-2(1H)-pyridinone

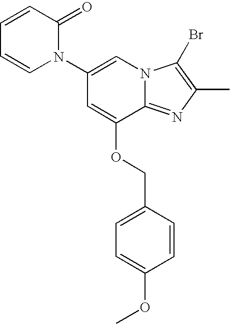

To a suspension of 1-[2-Methyl-8-({[4-(methyloxy)phenyl]methyl}oxy)imidazo[1,2-a]pyridin-6-yl]-2(1H)-pyridinone (Description 36, 1.9 g, 5.3 mmol) in ethanol (70 ml) at 0° C. was added N-bromosuccinimide (980 mg, 5.5 mmol) portionwise over 2 minutes. The resulting mixture was stirred at this temperature for 15 minutes then concentrated in vacuo. The residue was partitioned between water and ethyl acetate. The insoluble material was filtered off, washed with ethyl acetate and water then diluted in dichloromethane, dried (MgSO₄) and concentrated in vacuo to give the title compound as a white foam. The ethyl acetate and aqueous phases were separated and the aqueous phase was extracted with ethyl acetate. The combined organic phases were washed three times with a saturated aqueous sodium bicarbonate solution, dried (MgSO₄) and concentrated in vacuo to give the title compound. The two crops were combined to give 2.2 g (93%) of the title compound as a white foam which was used in the next step without further purification. MS (ES+ve): [M+H]⁺ at m/z 440 (C₂₁H₁₈⁷⁹BrN₃O₃ requires [M+H]⁺ at m/z 440).

### Description 40: 1-[3-ethenyl-2-methyl-8-({[4-(methyloxy)phenyl]methyl}oxy)imidazo[1,2-a]pyridin-6-yl]-2(1H)-pyridinone

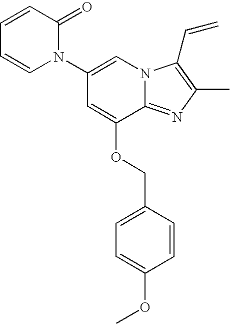

To a mixture of 1-[3-bromo-2-methyl-8-({[4-(methyloxy)phenyl]methyl}oxy)imidazo[1,2-a]pyridin-6-yl]-2(1H)-pyridinone (Description 39, 2.2 g, 4.9 mmol), potassium carbonate (2.0 g, 14.8 mmol) and tetrakis(triphenylphosphine)palladium(0) (850 mg, 0.7 mmol) was added toluene (30 ml) and ethanol (30 ml) and the resulting mixture was stirred 10 min at room temperature. Triethenylboroxin pyridinium complex (1.8 g, 7.4 mmol) was added and the resulting mixture was refluxed for 2 h then cooled to room temperature. The insoluble material was filtered off and washed with ethanol and most of the solvent was removed in vacuo. The residue was partitioned between ethyl acetate and water and the precipitate formed was filtered off, dissolved in dichloromethane, dried (MgSO₄) and concentrated in vacuo to give the title compound. The ethyl acetate and aqueous phases were separated and the aqueous phase was extracted with ethyl acetate. The combined organic phases were washed twice with a saturated aqueous sodium bicarbonate solution, dried (MgSO₄) and concentrated in vacuo. Purification of the residue by flash chromatography on silica gel (dichloromethane/methanol: 99/1 to 97/3) gave the title compound as a yellow solid. The two crops were combined to give 1.6 g (83%) of the title compound. MS (ES+ve): [M+H]⁺ at m/z 388 (C₂₃H₂₁N₃O₃ requires [M+H]⁺ at m/z 388).

### Description 41: 1-(3-ethenyl-8-hydroxy-2-methylimidazo[1,2-a]pyridin-6-yl)-2(1H)-pyridinone trifluoroacetic acid salt

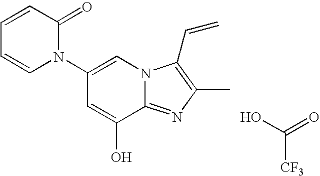

To a suspension of 1-[3-ethenyl-2-methyl-8-({[4-(methyloxy)phenyl]methyl}oxy)imidazo[1,2-a]pyridin-6-yl]-2(1H)-pyridinone (Description 40, 1.15 g, 3 mmol) in dichloromethane (10 ml) at room temperature was added trifluoroacetic acid (10 ml) dropwise and the resulting solution was stirred for 15 min then concentrated in vacuo. The residue was co-evaporated two times with dichloromethane then triturated with methanol. The insoluble material was filtered off and the mother liquors were removed in vacuo. The residue was triturated with ether to give the title compound (1.91 g, 79%) as a yellow solid. MS (ES+ve): [M+H]⁺ at m/z 268 (C₁₅H₁₃N₃O₂ requires [M+H]⁺ at m/z 268).

### Description 42: 8-{[(2,6-dimethylphenyl)methyl]oxy}-2-methyl-6-(2-oxo-1(2H)-pyridinyl)imidazo[1,2-a]pyridine-3-carbaldehyde

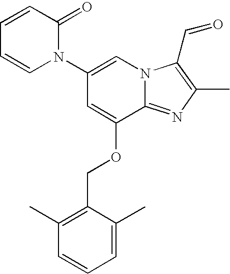

To a solution of 1-(8-{[(2,6-dimethylphenyl)methyl]oxy}-3-ethenyl-2-methylimidazo[1,2-a]pyridin-6-yl)-2(1H)-pyridinone (example 105, 310 mg, 0.8 mmol) in tetrahydrofuran (10 ml) were added sodium periodate (428 mg, 2 mmol), osmium tetroxide (4% in water, 780 μl, 0.12 mmol) and water (0.25 ml) and the resulting suspension was stirred at room temperature for 2 h. Sodium periodate (100 mg, 0.5 mmol) was added and the resulting mixture stirred for another 30 min. The precipitate was filtered off and washed with methanol and most of the solvent was removed in vacuo. The residue was partitioned between ethyl acetate and a saturated aqueous sodium bicarbonate solution. The insoluble material was filtered off, dissolved in dichloromethane, dried (MgSO₄) and concentrated in vacuo to give the title compound. The ethyl acetate and the saturated aqueous sodium bicarbonate solution were separated. The aqueous phase was extracted with ethyl acetate and the combined organic phases were washed with a saturated aqueous sodium bicarbonate solution, dried (MgSO₄) and concentrated in vacuo to give the title compound. The two crops were combined to give 310 mg (100%) of the title compound which was used in the next step without further purification. MS (ES+ve): [M+H]⁺ at m/z 388 (C₂₃H₂₁N₃O₃ requires [M+H]⁺ at m/z 388).

### Description 43. 1-(3-[(Dimethylamino)methyl]-8-{[(2,6-dimethylphenyl)methyl]oxy}-2-methylimidazo[1,2-a]pyridin-6-yl)-2(1H)-pyridinone

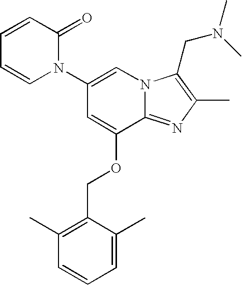

A mixture of 1-(8-{[(2,6-dimethylphenyl)methyl]oxy}-2-methylimidazo[1,2-a]pyridin-6-yl)-2(1H)-pyridinone (Description 38, 100 mg, 0.28 mmol), paraformaldehyde (10 mg, 0.31 mmol) and dimethylamine hydrochloride (25 mg, 0.31 mmol) in methanol (2 ml) was heated at reflux for 90 minutes. A second portion of paraformaldehyde (10 mg, 0.31 mmol) and dimethylamine hydrochloride (25 mg, 0.31 mmol) were added and heating continued for 60 minutes. The mixture was allowed to cool and was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane to afford the title compound MS (ES+ve): [M+H]⁺ at m/z 417 (C₂₅H₂₈N₄O₂ requires [M+H]⁺ at m/z 417).

### Description 44. [8-{[(2,6-Dimethylphenyl)methyl]oxy}-2-methyl-6-(2-oxo-1(2H)-pyridinyl)imidazo[1,2-a]pyridin-3-yl]-N,N,N-trimethylmethanaminium iodide

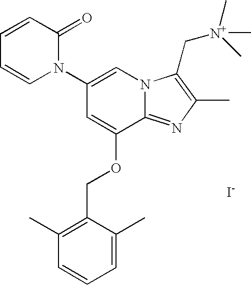

A mixture of 1-(3-[(dimethylamino)methyl]-8-{[(2,6-dimethylphenyl)methyl]oxy}-2-methylimidazo[1,2-a]pyridin-6-yl)-2(1H)-pyridinone (Description 43, 86 mg, 0.21 mmol) and iodomethane (15 μl, 0.23 mmol) in ethanol (2 ml) was stirred at room temperature for 18 hours. The resulting solid was collected by filtration and washed with diethyl ether to afford the title compound. Proton NMR (d6-DMSO) δ 8.70 (H, s), 7.80 (H, m), 7.61 (H, m), 7.25-7.12 (4H, m), 6.57 (H, m), 6.43 (H, m), 5.24 (2H, s), 4.91 (2H, s), 3.07 (9H, s), 2.43 (3H, s), 2.34 (6H, s).

### Description 45 N-[(2,6-Dimethylphenyl)methyl]-2,3-dimethyl-6-(4,4,5,5-tetramethyl-1,3,2-dioxaborolan-2-yl)imidazo[1,2-a]pyridin-8-amine

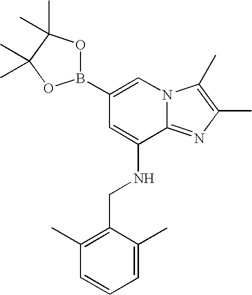

To a solution of 6-bromo-N-[(2,6-dimethylphenyl)methyl]-2,3-dimethylimidazo[1,2-a]pyridin-8-amine (200 mg, 0.56 mmol; WO98/37080) in dioxane (5 ml) was added diboron pinacol ester (168 mg, 0.66 mmol), 1,1′-bis(diphenylphosphino)ferrocene (16 mg, 0.029 mmol) and potassium acetate (164 mg, 1.7 mmol). After degassing the reaction mixture with argon, [1,1′-bis(diphenylphosphino)ferrocene]dichloropalladium (24 mg, 0.029 mmol) was added and the mixture heated to reflux for 4 hours. After cooling, the reaction mixture was diluted with ethyl acetate and water. The organic layer was dried (MgSO₄) and the solvent evaporated to yield the crude boronate ester. MS (ES+ve): [M+H]⁺ at m/z 406 (C₂₄H₃₂BN₃O₅ requires [M+H]⁺ at m/z 406).

### Description 46 2,3-Dimethyl-8-({[4-(methyloxy)phenyl]methyl}oxy)-6-(4,4,5,5-tetramethyl-1,3,2-dioxaborolan-2-yl)imidazo[1,2-a]pyridine

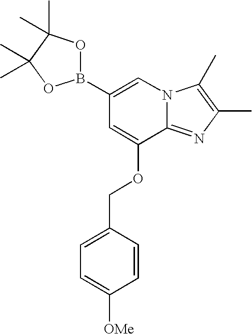

To a solution of 6-bromo-2,3-dimethyl-8-({[4-(methyloxy)phenyl]methyl}oxy)imidazo[1,2-a]pyridine (500 mg, 1.39 mmol; Description 21) in dioxane (20 ml) was added diboron pinacol ester (420 mg, 1.65 mmol), 1,1′-bis(diphenylphosphino)ferrocene (40 mg, 0.07 mmol) and potassium acetate (410 mg, 4.18 mmol). After degassing the reaction mixture with argon, [1,1′-bis(diphenylphosphino)ferrocene]dichloropalladium (60 mg, 0.07 mmol) was added and the mixture heated to reflux for 16 hours. After cooling, the reaction mixture was diluted with ethyl acetate and water. The organic layer was dried (MgSO₄) and the solvent evaporated to yield the crude boronate ester. MS (ES+ve): [M+H]⁺ at m/z 327 (boronic acid C₁₇H₁₉BN₂O₄ requires [M+H]⁺ at m/z 327).

### Description 47 2-[2,3-Dimethyl-8-({[4-(methyloxy)phenyl]methyl}oxy)imidazo[1,2-a]pyridin-6-yl]-3(2H)-pyridazinone

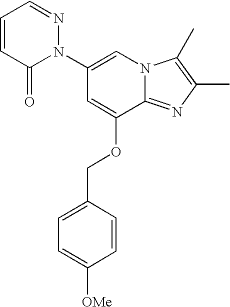

To a solution of 2,3-dimethyl-8-({[4-(methyloxy)phenyl]methyl}oxy)-6-(4,4,5,5-tetramethyl-1,3,2-dioxaborolan-2-yl)imidazo[1,2-a]pyridine (˜1.39 mmol; crude product from Description 46) in dichloromethane (20 ml) was added 3(2H)-pyridazinone (400 mg, 4.17 mmol), copper acetate (252 mg, 1.39 mmol), pyridine (112 ul, 1.39 mmol), triethylamine (193 ul, 1.39 mmol) and 4 Angstrom powdered molecular sieves (300 mg). The reaction mixture was stirred at room temperature for 10 days. The mixture was loaded onto an Isolute® SCX cartridge, eluting with methanol, then 2M NH₃ in methanol. After evaporation, the crude product was further purified by chromatography on silica gel (ethyl acetate/methanol) to give the title compound. MS (ES+ve): [M+H]⁺ at m/z 377 (C₂₁H₂₀N₄O₃ requires [M+H]⁺ at m/z 377).

### Description 48 2-(8-Hydroxy-2,3-dimethylimidazo[1,2-a]pyridin-6-yl)-3(2H)-pyridazinone

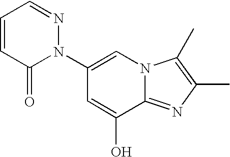

Trifluoroacetic acid (3 mL) was added to a solution of 2-[2,3-dimethyl-8-({[4-(methyloxy)phenyl]methyl}oxy)imidazo[1,2-a]pyridin-6-yl]-3(2H)-pyridazinone (255 mg, 0.68 mmol; Description 47) in dichloromethane (3 mL). The reaction mixture was stirred at room temperature for 30 minutes and then the solvent evaporated. The mixture was loaded onto an Isolute® SCX cartridge, eluting with methanol, then 2M NH₃ in methanol which, after evaporation, gave the title compound. MS (ES+ve): [M+H]⁺ at m/z 257 (C₁₃H₁₂N₄O₂ requires [M+H]⁺ at m/z 257).

Alternatively, the same compound can be synthesized according to the following procedure. A mixture of 6-bromo-2,3-dimethyl-8-({[4-(methyloxy)phenyl]methyl}oxy)imidazo[1,2-a]pyridine (4.02 g, 11 mmol; Description 21), 3(2H)-pyridazinone (1.28 g, 13 mmol), copper(I)iodide (423 mg, 2.2 mmol), potassium carbonate (3.07 g, 22 mmol) and N-methylpyrrolidinone (10 ml) was heated in an Initiator™ Microwave Synthesizer at 160° C. for 8 hours. The cooled reaction mixture was loaded onto an Isolute® SCX cartridge, washed with methanol and then eluted with 1M NH₃/MeOH. The solvent was evaporated and the residue purified by silica gel chromatography (methanol/dichloromethane) to give a mixture of the title compound and 2-[2,3-dimethyl-8-({[4-(methyloxy)phenyl]methyl}oxy)imidazo[1,2-a]pyridin-6-yl]-3(2H)-pyridazinone. The mixture was dissolved in dichloromethane (3 ml), treated with trifluoroacetic acid (3 ml) and stirred at room temperature overnight. The solvent was evaporated and the residue triturated with methanol then filtered. The filtrate was evaporated to give the title compound. MS (ES+ve): [M+H]⁺ at m/z 257 (C₁₃H₁₂N₄O₂ requires [M+H]⁺ at m/z 257).

### Description 49 Ethyl 1-(8-{[(2,6-Dimethylphenyl)methyl]amino}-2,3-dimethylimidazo[1,2-a]pyridin-6-yl)-1H-pyrazole-4-carboxylate

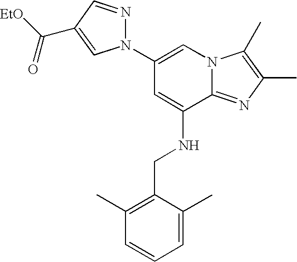

A mixture of 6-bromo-N-[(2,6-dimethylphenyl)methyl]-2,3-dimethylimidazo[1,2-a]pyridin-8-amine (320 mg, 0.89 mmol; WO98/37080), ethyl 1H-pyrazole-4-carboxylate (251 mg, 1.8 mmol), copper(I) iodide (51 mg, 0.27 mmol), trans-1,2-diaminocyclohexane (31 mg, 0.27 mmol), potassium phosphate (682 mg, 3.2 mmol) and dioxane (0.5 mL) was heated in an Initiator™ Microwave Synthesizer at 160° C. for 18 hours. The cooled mixture was partitioned between ethyl acetate and water. The organic phase was washed with water, then brine, and then evaporated. The residue was purified by chromatography on silica gel (ethyl acetate/hexane) to give the title compound. MS (ES+ve): [M+H]⁺ at m/z 418 (C₂₄H₂₇N₅O₂ requires [M+H]⁺ at m/z 418).

### Description 50. Phenylmethyl 1-(8-{[(2,6-dimethylphenyl)methyl]amino}-2,3-dimethylimidazo[1,2-a]pyridin-6-yl)-6-oxo-1,6-dihydro-3-pyridinecarboxylate

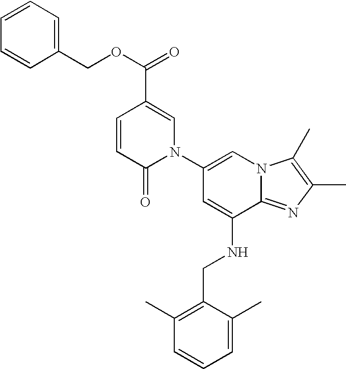

A mixture of 6-bromo-N-[(2,6-dimethylphenyl)methyl]-2,3-dimethylimidazo[1,2-a]pyridin-8-amine (300 mg, 0.837 mmol; WO98/37080), phenylmethyl 6-hydroxy-3-pyridinecarboxylate (192 mg, 0.837 mmol), copper(I) iodide (33 mg, 0.173 mmol), potassium phosphate (354 mg, 1.67 mol), N,N′-dimethylethylenediamine (30 mg, 0.34 mmol) and dioxane (4 ml) was heated in an Initiator™ Microwave Synthesizer at 160° C. for 12 hours. The cooled reaction mixture was partitioned between ethyl acetate and dilute aqueous ammonia, The organic phase was washed with water, then brine, dried (MgSO₄) and evaporated. Purification by chromatography on silica gel (ethyl acetate/toluene) gave the title compound as a pale yellow solid.

### Description 51. 1-(8-{[(2,6-Dimethylphenyl)methyl]amino}-2,3-dimethylimidazo[1,2-a]pyridin-6-yl)-6-oxo-1,6-dihydro-3-pyridinecarboxylic acid hydrochloride

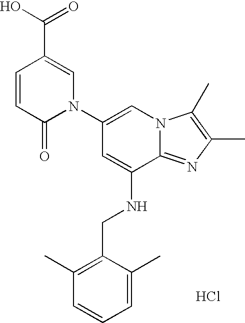

To a solution of phenylmethyl 1-(8-{[(2,6-dimethylphenyl)methyl]amino}-2,3-dimethylimidazo[1,2-a]pyridin-6-yl)-6-oxo-1,6-dihydro-3-pyridinecarboxylate (125.6 mg, 0.248 mmol; Description 50) in methanol (10 ml) was added water (3 ml) and 2N NaOH (2 ml). The mixture was heated under reflux for 2 hours. The cooled reaction mixture was diluted with water, and then acidified with 2N HCl. The mixture was loaded onto a 10 g tC18 Sep-Pak® cartridge and eluted with a gradient up to methanol/water/2N HCl (65:35:0.5) to give the title compound as a cream solid. MS (ES+ve): [M+H]⁺ at m/z 417 (C₂₄H₂₄N₄O₃ requires [M+H]⁺ at m/z 417).

### Description 52: 8-bromo-2,3-dimethyl-6-(1H-1,2,4-triazol-1-yl)imidazo[1,2-a]pyridine

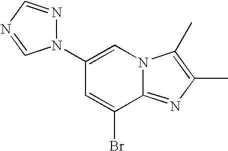

A mixture of 6,8-dibromo-2,3-dimethylimidazo[1,2-a]pyridine (Description 1; 2.77 g, 9.12 mmol), 1,2,4-triazole (0.623 g, 9.12 mmol), copper(I) iodide (89 mg, 0.467 mmol), cesium carbonate (6.24 g, 19.15 mmol) and trans N,N′ dimethyl-1,2-cyclohexanediamine (194 mg, 1.36 mmol) in dimethylformamide (10 mL) was heated in an Initiator™ Microwave Synthesizer at 120° C. for 16 hours. The cooled mixture was diluted with dichloromethane (15 mL) and filtered. The filtrate was evaporated and the residue was loaded onto an Isolute® SCX cartridge, eluting with methanol, then 2M NH₃ in methanol. Fractions containing the product were purified by silica gel chromatography. Elution with dichloromethane/methanol (1% and increasing the polarity to 6%) gave 6-bromo-2,3-dimethyl-8-(1H-1,2,4-triazol-1-yl)imidazo[1,2-a]pyridine and the title compound as a colourless foam MS (ES+ve): [M+H]⁺ at m/z 293 (C₁₁H₁₀BrN₇ requires [M+H]⁺ at m/z 293).

### Description 53. 6-bromo-N-[(2-methylphenyl)methyl]-2,3-dimethylimidazo[1,2-a]pyridin-8-amine

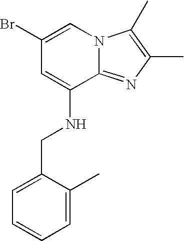

A mixture of 6-bromo-2,3-dimethylimidazo[1,2-a]pyridin-8-amine (300 mg, 1.25 mmol; WO98/37080), 1-(bromomethyl)-2-methylbenzene (0.20 ml, 1.5 mmol) and sodium carbonate (420 mg, 4.0 mmol) were stirred in dimethylformamide (10 ml) for 42 hours, the solvent was evaporated and the residue partitioned between ethyl acetate and water. The organic phase was washed with water, brine, dried (MgSO₄) and after evaporation the residue was purified by chromatography on silica gel (ethyl acetate/hexane) and evaporated to give the title compound. MS (ES+ve): [M+H]⁺ at m/z 344 and 346 (C₁₇H₁₈BrN₃ requires [M+H]⁺ at m/z 344 and 346).

### Description 54. 2,3-dimethyl-6-(1H-1,2,4-triazol-1-yl)imidazo[1,2-a]pyridin-8-amine

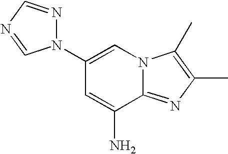

A mixture of 6-bromo-2,3-dimethylimidazo[1,2-a]pyridin-8-amine (WO05/077949, 4.0 g, 16.7 mmol) in NMP (10 ml) was added copper (I) iodide (317 mg, 1.67 mmol), potassium carbonate (4.6 g, 33.4 mmol), 1,2,4-triazole (2.3 g, 33.4 mmol) N,N′-dimethyl-1,2-ethanediamine (0.117 mL, 1.67 mmol) was heated in an Initiator™ Microwave Synthesizer at 200° C. for 1 hour. The reaction mixture was loaded onto an Isolute® SCX cartridge, eluted with methanol, then 1M NH₃ in methanol which gave, after evaporation, the product which was further purified by chromatography on silica gel (methanol/dichloromethane) and evaporated to give the title compound. MS (ES+ve): [M+H]⁺ at m/z 229 (C₁₁H₁₂N₆ requires [M+H]⁺ at m/z 229).

### Description 55. 1-(8-bromo-2,3-dimethylimidazo[1,2-a]pyridin-6-yl)-2(1H)-pyridinone

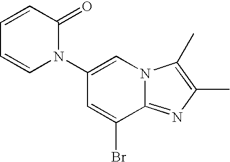

6,8-dibromo-2,3-dimethylimidazo[1,2-a]pyridine (3.72 g, 12.2 mmol, Description 1), 2-hydroxypyridine (2.32 g, 24.4 mmol), cesium carbonate (8.35 g, 25.6 mmol), copper(I) iodide (115 mg, 0.6 mmol) and (1S,2S)-N,N-dimethyl-1,2-cyclohexanediamine (255 mg, 1.8 mmol) were combined in dimethylformamide (120 mL) and stirred at 120° C. for 72 hours. The mixture was loaded onto an Isolute® SCX cartridge, washing with methanol and then eluted with 2M NH₃/MeOH. Solvent was removed in vacuo from appropriate fractions and the resulting mixture was purified on silica eluting with ethyl acetate. Evaporation followed by trituration with diethyl ether gave the title compound. MS (ES+ve): [M+H]⁺ at m/z 318/320 (C₁₄H₁₂N₃OBr requires [M+H]⁺ at m/z 318/320).

### Description 56. 6-bromo-N-[(2-ethylphenyl)methyl]-2,3-dimethylimidazo[1,2-a]pyridin-8-amine

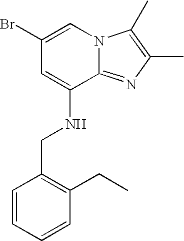

A mixture of 6-bromo-2,3-dimethylimidazo[1,2-a]pyridin-8-amine (WO05/077949; 94 mg, 0.39 mmol), sodium carbonate (132 mg, 1.25 mmol), (2-ethylphenyl)methyl methanesulfonate (crude mixture, 1.17 mmol) and potassium iodide (20 mg, 0.12 mmol) in anhydrous dimethylformamide (5 mL) was stirred at ambient temperature for 48 hours. The mixture was partitioned between ethyl acetate and water, the organic layer evaporated and purified by MDAP. Fractions containing the product were combined and evaporated. The product was re-dissolved into ethyl acetate, washed with sodium bicarbonate and water and the combined organic solvents were evaporated to give the title compound. MS (ES+ve): [M+H]⁺ at m/z 358/360 (C₁₈H₂₀N₃Br requires [M+H]⁺ at m/z 358/360).

### EXAMPLE 1

### 8-{[(2,6-Dimethylphenyl)methyl]oxy}-2,3-dimethyl-6-phenylimidazo[1,2-a]pyridine

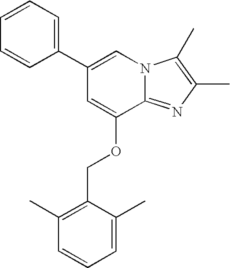

A mixture of 6-bromo-8-{[(2,6-dimethylphenyl)methyl]oxy}-2,3-dimethylimidazo[1,2-a]pyridine (50 mg, 0.139 mmol; Description 2), phenylboronic acid (21 mg, 0.172 mmol), [1,1′-bis(diphenylphosphino)ferrocene]dichloropalladium (2 mg, 0.0038 mmol), 2M aqueous sodium carbonate (0.2 mL) and dimethylformamide (0.3 mL) was heated in an Initiator™ Microwave Synthesizer at 100° C. for 30 minutes. The cooled mixture was partitioned between ethyl acetate and water. The organic phase was washed with water, then brine, dried (MgSO₄) and evaporated. Purification by chromatography on silica gel (ethyl acetate/hexane) gave the title compound. ¹H NMR (DMSO-d₆) δ 2.26 (s, 3H), 2.38 (s, 6H), 2.43 (s, 3H), 5.34 (s, 2H), 7.12 (m, 3H), 7.21 (m, 1H), 7.36 (m, 1H), 7.51 (m, 2H), 7.83 (dt, 2H), 8.06 (d, 1H); MS (ES+ve): [M+H]⁺ at m/z 357 (C₂₄H₂₄N₂O requires [M+H]⁺ at m/z 357).

### EXAMPLE 2

### 8-{[(2,6-Dimethylphenyl)methyl]oxy}-2,3-dimethyl-6-(3-pyridinyl)imidazo[1,2-a]pyridine

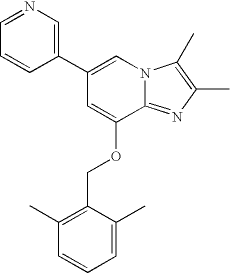

A mixture of 6-bromo-8-{[(2,6-dimethylphenyl)methyl]oxy}-2,3-dimethylimidazo[1,2-a]pyridine (50 mg, 0.139 mmol; Description 2), 3-pyridinylboronic acid (44 mg, 0.358 mmol), [1,1′-bis(diphenylphosphino)ferrocene]dichloropalladium (3 mg, 0.0057 mmol), 2M aqueous sodium carbonate (0.2 mL) and dimethylformamide (0.3 mL) was heated in an Initiator™ Microwave Synthesizer at 100° C. for 1 hour. The cooled mixture was partitioned between ethyl acetate and water. The organic phase was washed with water, then brine, dried (MgSO₄) and evaporated. Purification by chromatography on silica gel (ethyl acetate/2M ammonia in methanol) gave the title compound. MS (ES+ve): [M+H]⁺ at m/z 358 (C₂₃H₂₃N₃O requires [M+H]⁺ at m/z 358).

### EXAMPLE 3

### 8-{[(2,6-Dimethylphenyl)methyl]oxy}-2,3-dimethyl-6-[2-(methyloxy)-3-pyridinyl]imidazo[1,2-a]pyridine

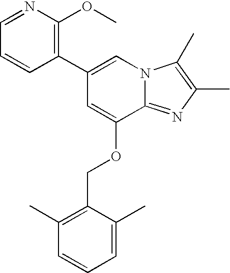

A mixture of 6-bromo-8-{[(2,6-dimethylphenyl)methyl]oxy}-2,3-dimethylimidazo[1,2-a]pyridine (50 mg, 0.139 mmol; Description 2), [2-(methyloxy)-3-pyridinyl]boronic acid (30 mg, 0.196 mmol), [1,1′-bis(diphenylphosphino)ferrocene]dichloropalladium (2 mg, 0.0038 mmol), 2M aqueous sodium carbonate (0.2 mL) and dimethylformamide (0.3 mL) was heated in an Initiator™ Microwave Synthesizer at 100° C. for 1 hour. The cooled mixture was partitioned between ethyl acetate and water. The organic phase was washed with water, then brine, dried (MgSO₄) and evaporated. Purification was by chromatography on silica gel (ethyl acetate). After chromatography the residual gum was triturated from diethyl ether/hexane to give the title compound. MS (ES+ve): [M+H]⁺ at m/z 388 (C₂₄H₂₅N₃O₂ requires [M+H]⁺ at m/z 388).

### EXAMPLE 4

### 8-{[(2,6-Dimethylphenyl)methyl]oxy}-6-(2-furanyl)-2,3-dimethylimidazo[1,2-a]pyridine

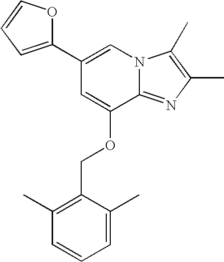

A mixture of 6-bromo-8-{[(2,6-dimethylphenyl)methyl]oxy}-2,3-dimethylimidazo[1,2-a]pyridine (50 mg, 0.139 mmol; Description 2), 2-furanylboronic acid (25 mg, 0.223 mmol), [1,1′-bis(diphenylphosphino)ferrocene]dichloropalladium (2 mg, 0.0038 mmol), 2M aqueous sodium carbonate (0.2 mL) and dimethylformamide (0.3 mL) was heated in an Initiator™ Microwave Synthesizer at 100° C. for 1 hour. The cooled mixture was partitioned between ethyl acetate and water. The organic phase was washed with water, then brine, dried (MgSO₄) and evaporated. Purification was by chromatography on silica gel (ethyl acetate/toluene) to give the title compound. MS (ES+ve): [M+H]⁺ at m/z 347 (C₂₂H₂₂N₂O₂ requires [M+H]⁺ at m/z 347).

### EXAMPLE 5

### N-[(2-Ethyl-6-methylphenyl)methyl[-2,3-dimethyl-6-(3-methyl-1,2,4-oxadiazol-5-yl)imidazo[1,2-a]pyridin-8-amine hydrochloride

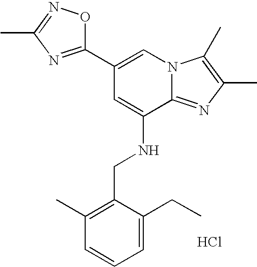

To a solution of sodium 8-{[(2-ethyl-6-methylphenyl)methyl]amino}-2,3-dimethylimidazo[1,2-a]pyridine-6-carboxylate (50 mg, 0.139 mmol; Description 8) in dimethylformamide (2 mL) was added N-hydroxyacetamidine (12 mg, 0.16 mmol), 1-hydroxybenzotriazole (22 mg, 0.16 mmol) and 1-(3-dimethylaminopropyl)-3-ethylcarbodiimide hydrochloride (34 mg, 0.18 mmol). The reaction mixture was stirred at room temperature for 20 hours. The mixture was applied to an Isolute® SCX cartridge, elution with methanol, then 2M NH₃ in methanol gave N-{[(8-{[(2-ethyl-6-methylphenyl)methyl]amino}-2,3-dimethylimidazo[1,2-a]pyridin-6-yl)carbonyl]oxy}ethanimidamide (58 mg); MS (ES+ve): [M+H]⁺ at m/z 394 (C₂₂H₂₇N₅O₂ requires [M+H]⁺ at m/z 394). Toluene (5 mL) was added to the intermediate and the mixture heated under Dean and Stark conditions for 4 hours. After cooling the solvent was evaporated, the residue was dissolved in methanol, and then applied to an Isolute® PE-AX cartridge. Elution with methanol gave the crude product. Elution with 10% acetic acid in methanol allowed the recovery of 8-{[(2-ethyl-6-methylphenyl)methyl]amino}-2,3-dimethylimidazo[1,2-a]pyridine-6-carboxylic acid. The crude product was further purified by chromatography on silica gel (ethyl acetate/hexane). The purified product (12 mg) was dissolved in dichloromethane (3 mL), and then 1M HCl in diethyl ether (0.320 mL) was added. After 4 hours the solvents were evaporated, and the residue was dissolved in water, then freeze dried to give the title compound. MS (ES+ve): [M+H]⁺ at m/z 376 (C₂₂H₂₅N₅O requires [M+H]⁺ at m/z 376).

### EXAMPLE 6

### N-[(2-Ethyl-6-methylphenyl)methyl]-2,3-dimethyl-6-(5-methyl-1,3,4-oxadiazol-2-yl)imidazo[1,2-a]pyridin-8-amine hydrochloride

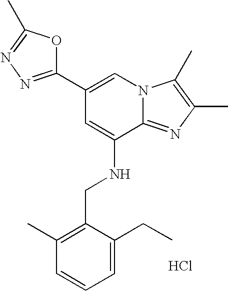

A suspension of 8-{[(2-ethyl-6-methylphenyl)methyl]amino}-2,3-dimethylimidazo[1,2-a]pyridine-6-carbohydrazide (128 mg, 0.36 mmol—Description 9) in triethyl orthoacetate (2 mL) was stirred at 140° C. for 2 h. The solution was cooled and the resulting solid collected by filtration. The solid was dissolved in dichloromethane (2 mL), and then 1M HCl in diethyl ether (1 mL) was added. After 2 hours the solvents were evaporated to give the title compound. MS (ES+ve): [M+H]⁺ at m/z 376 (C₂₂H₂₅N₅O requires [M+H]⁺ at m/z 376).

### EXAMPLE 7

### N-[(2,6-Dimethylphenyl)methyl]-2,3-dimethyl-6-phenylimidazo[1,2-a]pyridin-8-amine

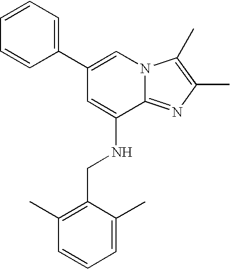

A mixture of 6-bromo-N-[(2,6-dimethylphenyl)methyl]-2,3-dimethylimidazo[1,2-a]pyridin-8-amine (49 mg, 0.137 mmol; WO 98/37080), phenylboronic acid (33 mg, 0.274 mmol), tetrakis(triphenylphosphine)palladium (9 mg, 0.0082 mmol), potassium tert-butoxide (167 mg, 1.37 mmol), 1,2-dimethoxyethane (1 mL) and water (0.3 mL) was heated in an Initiator™ Microwave Synthesizer at 100° C. for 1 hour. The cooled mixture was subjected to MDAP purification. Fractions containing the product were loaded onto an Isolute® SCX cartridge, elution with methanol, then 1M NH₃ in methanol gave the title compound. ¹H NMR (MeOD-d₄) δ 2.29 (s, 3H), 2.40 (s+s, 9H), 4.45 (s, 2H), 6.61 (s, 1H), 7.09 (m, 3H), 7.36 (m, 1H), 7.46 (m, 2H), 7.60 (m, 1H), 7.69 (m, 2H); MS (ES+ve): [M+H]⁺ at m/z 356 (C₂₄H₂₅N₃ requires [M+H]⁺ at m/z 356).

### EXAMPLE 8

### N-[(2,6-Dimethylphenyl)methyl]-2,3-dimethyl-6-(3-methylphenyl)imidazo[1,2-a]pyridin-8-amine formate

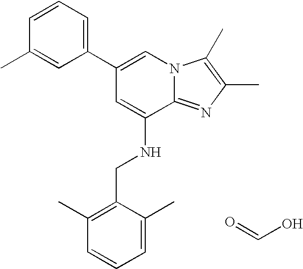

A mixture of 6-bromo-N-[(2,6-dimethylphenyl)methyl]-2,3-dimethylimidazo[1,2-a]pyridin-8-amine (139 mg, 0.388 mmol; WO 98/37080), (3-methylphenyl)boronic acid (106 mg, 0.779 mmol), tetrakis(triphenylphosphine)palladium (27 mg, 0.0246 mmol), potassium tert-butoxide (475 mg, 3.90 mmol), 1,2-dimethoxyethane (1.2 mL) and water (0.4 mL) was heated in an Initiator™ Microwave Synthesizer at 100° C. for 45 minutes. The solvent was evaporated under reduced pressure and the residue partitioned between dichloromethane and water. The organic layer was evaporated, then redissolved in methanol and loaded onto an Isolute® SCX cartridge. The cartridge was eluted with methanol, then 1M NH₃ in methanol. The product was further purified by MDAP to give the title compound. MS (ES+ve): [M+H]⁺ at m/z 370 (C₂₅H₂₇N₃ requires [M+H]⁺ at m/z 370).

### EXAMPLE 9

### N-[(2,6-Dimethylphenyl)methyl]-2,3-dimethyl-6-[2-(methyloxy)phenyl]imidazo[1,2-a]pyridin-8-amine formate

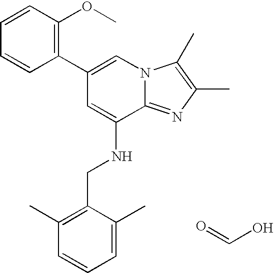

A mixture of 6-bromo-N-[(2,6-dimethylphenyl)methyl]-2,3-dimethylimidazo[1,2-a]pyridin-8-amine (105 mg, 0.293 mmol; WO 98/37080), [2-(methyloxy)phenyl]boronic acid (89 mg, 0.586 mmol), tetrakis(triphenylphosphine)palladium (20 mg, 0.018 mmol), potassium tert-butoxide (358 mg, 2.94 mmol), 1,2-dimethoxyethane (1.2 mL) and water (0.4 mL) was heated in an Initiator™ Microwave Synthesizer at 100° C. for 45 minutes. The solvent was evaporated under reduced pressure and the residue partitioned between dichloromethane and water. The organic layer was evaporated, then redissolved in methanol and loaded onto an Isolute® SCX cartridge. The cartridge was eluted with methanol, then 1M NH₃ in methanol. The product was further purified by MDAP to give the title compound. MS (ES+ve): [M+H]⁺ at m/z 386 (C₂₅H₂₇N₃ O requires [M+H]⁺ at m/z 386).

### EXAMPLE 10

### 6-(3,5-Dimethyl-4-isoxazolyl)-N-[(2,6-dimethylphenyl)methyl]-2,3-dimethylimidazo[1,2-a]pyridin-8-amine hydrochloride

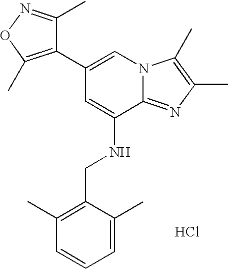

A mixture of 6-bromo-N-[(2,6-dimethylphenyl)methyl]-2,3-dimethylimidazo[1,2-a]pyridin-8-amine (116 mg, 0.324 mmol; WO 98/37080), (3,5-dimethyl-4-isoxazolyl)boronic acid (137 mg, 0.972 mmol), tetrakis(triphenylphosphine)palladium (22 mg, 0.02 mmol), potassium tert-butoxide (396 mg, 3.25 mmol), 1,2-dimethoxyethane (3 mL) and water (1 mL) was heated in an Initiator™ Microwave Synthesizer at 100° C. for 50 minutes. The reaction mixture was loaded onto an Isolute® SCX cartridge, elution with methanol, then 1M NH₃ in methanol gave the impure product that was further purified by chromatography on silica gel (dichloromethane/methanol). The purified solid was dissolved in dichloromethane (1 mL), and then 1M HCl in diethyl ether (1 mL) was added. The solvents were evaporated to give the title compound. MS (ES+ve): [M+H]⁺ at m/z 375 (C₂₃H₂₆N₄ O requires [M+H]⁺ at m/z 375).

### EXAMPLE 11

### N-[(2,6-Dimethylphenyl)methyl]-2,3-dimethyl-6-(3-pyridinyl)imidazo[1,2-a]pyridin-8-amine hydrochloride

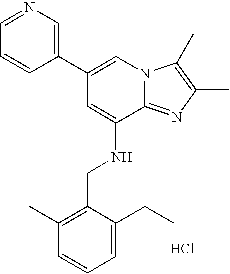

A mixture of 6-bromo-N-[(2,6-dimethylphenyl)methyl]-2,3-dimethylimidazo[1,2-a]pyridin-8-amine (344 mg, 0.96 mmol; WO 98/37080), 3-pyridinylboronic acid (300 mg, 2.44 mmol), [1,1′-bis(diphenylphosphino)ferrocene]dichloropalladium (20 mg, 0.038 mmol), 2M aqueous sodium carbonate (1.5 ml) and dimethylformamide (2.2 ml) was heated in an Initiator™ Microwave Synthesizer at 100° C. for 1 hour. The cooled mixture was partitioned between ethyl acetate and water. The organic phase was washed with water, then brine, dried (MgSO₄) and evaporated. Purification by chromatography on silica gel (ethyl acetate) gave the free base of the title compound as a gum. This was dissolved in methanol (5 ml) and water (1 ml), then 2M aqueous HCl (2 ml) was added. After stirring for 10 minutes the solvents were evaporated. The residue was redissolved in methanol and evaporated to give the title compound as a buff solid (87.7 mg). MS (ES+ve): [M+H]⁺ at m/z 357 (C₂₃H₂₄N₄ requires [M+H]⁺ at m/z 357).

### EXAMPLE 12

### N-[(2,6-Dimethylphenyl)methyl]-2,3-dimethyl-6-(4-pyridinyl)imidazo[1,2-a]pyridin-8-amine hydrochloride

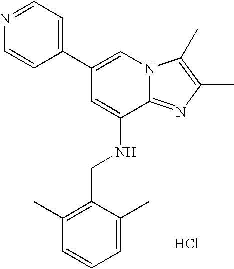

A mixture of 6-bromo-N-[(2,6-dimethylphenyl)methyl]-2,3-dimethylimidazo[1,2-a]pyridin-8-amine (70 mg, 0.20 mmol; WO 98/37080), 4-pyridinylboronic acid (48 mg, 0.40 mmol), [1,1′-bis(diphenylphosphino)ferrocene]dichloropalladium (5 mg, 0.0095 mmol), 2M aqueous sodium carbonate (1 mL) and dimethylformamide (2 mL) was heated in an Initiator™ Microwave Synthesizer at 100° C. for 1 hour. The cooled reaction mixture was loaded onto an Isolute® SCX cartridge, elution with methanol, then 2M NH₃ in methanol gave the impure product that was further purified by chromatography on silica gel (ethyl acetate). The purified solid was dissolved in methanol (2 mL), and then 1M HCl in diethyl ether (1 mL) was added. After stirring for 1 hour, the solvents were evaporated. The residue was dissolved in water and freeze dried to give the title compound as a yellow solid. (MS (ES+ve): [M+H]⁺ at m/z 357 (C₂₃H₂₄N₄ requires [M+H]⁺ at m/z 357).

### EXAMPLE 13

### N-[(2,6-Dimethylphenyl)methyl]-6-(2-furanyl)-2,3-dimethylimidazo[1,2-a]pyridin-8-amine hydrochloride

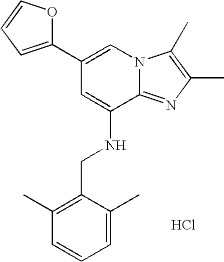

A mixture of 6-bromo-N-[(2,6-dimethylphenyl)methyl]-2,3-dimethylimidazo[1,2-a]pyridin-8-amine (50 mg, 0.14 mmol; WO 98/37080), 2-furanylboronic acid (25 mg, 0.22 mmol), [1,1′-bis(diphenylphosphino)ferrocene]dichloropalladium (5 mg, 0.0095 mmol), 2M aqueous sodium carbonate (1 mL) and dimethylformamide (2 mL) was heated in an Initiator™ Microwave Synthesizer at 100° C. for 1 hour. The cooled reaction mixture was loaded onto an Isolute® SCX cartridge, elution with methanol, then 2M NH₃ in methanol gave the impure product that was further purified by chromatography on silica gel (ethyl acetate/hexane). The purified solid was dissolved in methanol (2 mL), and then 1M HCl in diethyl ether (1 mL) was added. After stirring for 1 hour, the solvents were evaporated. The residue was dissolved in water and freeze dried to give the title compound as an off-white solid.

(MS (ES+ve): [M+H]⁺ at m/z 346 (C₂₂H₂₃N₃O requires [M+H]⁺ at m/z 346).

### EXAMPLE 14

### N-[(2,6-Dimethylphenyl)methyl]-2,3-dimethyl-6-(1H-pyrazol-1-yl)imidazo[1,2-a]pyridin-8-amine hydrochloride

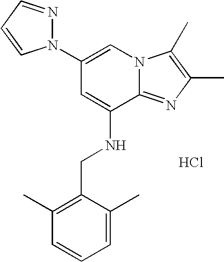

A mixture of 6-bromo-N-[(2,6-dimethylphenyl)methyl]-2,3-dimethylimidazo[1,2-a]pyridin-8-amine (400 mg, 1.12 mmol; WO 98/37080), 1H-pyrazole (152 mg, 2.23 mmol), copper(I) iodide (64 mg, 0.34 mmol), trans-1,2-cyclohexanediamine (40 μL, 0.34 mmol), potassium phosphate (853 mg, 4.02 mmol) and dry dioxane (16 mL) was heated at reflux for 3 days. The cooled mixture was diluted with methanol and filtered. The filtrate was then loaded onto an Isolute® SCX cartridge, and elution with methanol, then 2M NH₃ in methanol gave the impure product which was further purified by chromatography on silica gel (ethyl acetate/hexane). The purified solid was dissolved in dichloromethane (2 mL), and then 1M HCl in diethyl ether (1 mL) was added. After stirring for 30 minutes, the solvents were evaporated to give the title compound as a solid 145 mg (34%). MS (ES+ve): [M+H]⁺ at m/z 346 (C₂₁H₂₃N₅ requires [M+H]⁺ at m/z 346).

### EXAMPLE 15

### 8-{[(2,6-Dimethylphenyl)methyl]oxy}-2,3-dimethyl-6-(2-pyridinyl)imidazo[1,2-a]pyridine hydrochloride

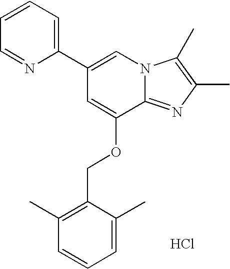

A mixture of 6-bromo-8-{[(2,6-dimethylphenyl)methyl]oxy}-2,3-dimethylimidazo[1,2-a]pyridine (100 mg, 0.279 mmol; Description 2), 2-pyridineboronic acid N-phenyldiethanolamine ester (149 mg, 0.557 mmol), tri(o-tolyl)phosphine (17 mg, 0.056 mmol), palladium(II) acetate (3 mg, 0.014 mmol), copper(I) iodide (21 mg, 0.111 mmol), potassium carbonate (77 mg, 0.557 mol), and tetrahydrofuran (1 mL) was heated in an Initiator™ Microwave Synthesizer at 100° C. for 3 hours. The cooled mixture was partitioned between ethyl acetate and water. The organic phase was washed with water, then brine, dried (MgSO₄) and evaporated. The residue was purified by chromatography on silica gel (ethyl acetate). The product was dissolved in methanol/water/2N HCl (2.5:50:2 mL) and then loaded onto a 10 g tC18 Sep-Pak® cartridge and eluted with a gradient up to methanol/water/2N HCl (70:30:0.5). Evaporation gave the title compound as a white solid. MS (ES+ve): [M+H]⁺ at m/z 358 (C₂₃H₂₃N₃O requires [M+H]⁺ at m/z 358).

### EXAMPLE 16

### N-[(2,6-Dimethylphenyl)methyl]-2,3-dimethyl-6-(2-pyridinyl)imidazo[1,2-a]pyridin-8-amine hydrochloride

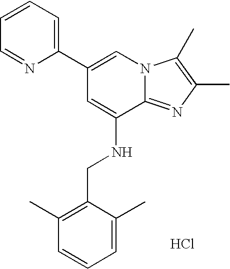

A mixture of 6-bromo-N-[(2,6-dimethylphenyl)methyl]-2,3-dimethylimidazo[1,2-a]pyridin-8-amine (100 mg, 0.279 mmol; WO 98/37080), 2-pyridineboronic acid N-phenyldiethanolamine ester (149 mg, 0.557 mmol), tri(o-tolyl)phosphine (17 mg, 0.056 mmol), palladium(II) acetate (3 mg, 0.014 mmol), copper(I) iodide (21 mg, 0.111 mmol), potassium carbonate (77 mg, 0.557 mol), and tetrahydrofuran (1 mL) was heated in an Initiator™ Microwave Synthesizer at 100° C. for 3 hours. The cooled mixture was partitioned between ethyl acetate and water. The organic phase was washed with water, then brine, dried (MgSO₄) and evaporated. The residue was purified by chromatography on silica gel (ethyl acetate/hexane). The purified solid was dissolved in dichloromethane (2 mL), and then 1M HCl in diethyl ether (1 mL) was added. After stirring for 2 hours, the solvents were evaporated to give the title compound as a brown solid. MS (ES+ve): [M+H]⁺ at m/z 357 (C₂₃H₂₄N₄ requires [M+H]⁺ at m/z 357).

### EXAMPLE 17

### N-[(2-Ethyl-6-methylphenyl)methyl]-2,3-dimethyl-6-(1H-1,2,4-triazol-3-yl)imidazo[1,2-a]pyridin-8-amine hydrochloride

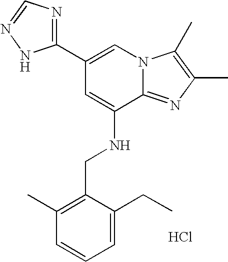

A mixture of N-[(dimethylamino)methylidene]-8-{[(2-ethyl-6-methylphenyl)methyl]amino}-2,3-dimethylimidazo[1,2-a]pyridine-6-carboxamide (40.2 mg, 0.103 mmol; Description 10), hydrazine hydrate (0.01 mL, 0.20 mmol) and acetic acid (1.6 mL) was heated at 90° C. for 2 hours. After cooling, the mixture was evaporated to dryness. The residue was purified by chromatography on silica gel (ethyl acetate/methanol). The purified solid was dissolved in methanol (3 mL), 2N HCl was added. After stirring for 5 minutes, the mixture was evaporated to give the title compound as a white solid. MS (ES+ve): [M+H]⁺ at m/z 361 (C₂₁H₂₄N₆ requires [M+H]⁺ at m/z 361).

### EXAMPLE 18

### N-[(2,6-Dimethylphenyl)methyl]-2,3-dimethyl-6-[2-(methyloxy)-3-pyridinyl]imidazo[1,2-a]pyridin-8-amine

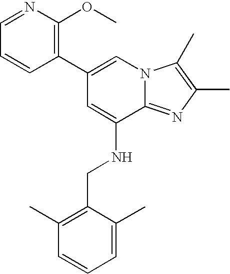

A mixture of 6-bromo-N-[(2,6-dimethylphenyl)methyl]-2,3-dimethylimidazo[1,2-a]pyridin-8-amine (200 mg, 0.558 mmol; WO 98/37080), [2-(methyloxy)-3-pyridinyl]boronic acid (120 mg, 0.785 mmol), [1,1′-bis(diphenylphosphino)ferrocene]dichloropalladium (8 mg, 0.0152 mmol), 2M aqueous sodium carbonate (0.6 mL) and dimethylformamide (1 mL) was heated in an Initiator™ Microwave Synthesizer at 100° C. for 1 hour. The cooled mixture was partitioned between ethyl acetate and water. The organic phase was washed with water, then brine, dried (MgSO₄) and evaporated. Purification by chromatography on silica gel (ethyl acetate/hexane) gave the title compound as a buff solid. MS (ES+ve): [M+H]⁺ at m/z 387 (C₂₄H₂₆N₄O requires [M+H]⁺ at m/z 387).

### EXAMPLE 19

### N-[(2-Ethyl-6-methylphenyl)methyl]-2,3-dimethyl-6-(1H-tetrazol-5-yl)imidazo[1,2-a]pyridin-8-amine hydrochloride

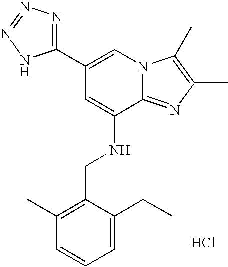

A mixture of 8-{[(2-ethyl-6-methylphenyl)methyl]amino}-2,3-dimethylimidazo[1,2-a]pyridine-6-carbonitrile (114 mg, 0.358 mmol; Description 11), sodium azide (72 mg, 1.11 mmol) and ammonium chloride (59 mg, 1.10 mmol) in dimethylformamide (2 mL) was heated at 130° C. for 5 hours. The cooled mixture was added to a mixture of water (40 mL), methanol (10 mL) and 2N HCl (3 mL), and then loaded onto a 10 g tC18 Sep-Pak® cartridge and eluted with a gradient up to methanol/water/2N HCl (80:20:0.5). Evaporation gave the title compound as a colourless solid. MS (ES+ve): [M+H]⁺ at m/z 362 (C₂₀H₂₃N₇ requires [M+H]⁺ at m/z 362).

### EXAMPLE 20

### N-[(2,6-Dimethylphenyl)methyl]-6-(1H-imidazol-1-yl)-2,3-dimethylimidazo[1,2-a]pyridin-8-amine hydrochloride

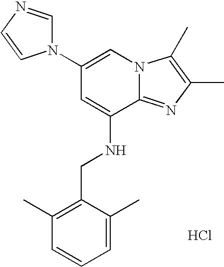

A mixture of 6-bromo-N-[(2,6-dimethylphenyl)methyl]-2,3-dimethylimidazo[1,2-a]pyridin-8-amine (50 mg, 0.14 mmol; WO 98/37080), imidazole (95 mg, 1.4 mmol), copper(I) iodide (2.7 mg, 0.014 mmol), trans-1,2-diaminocyclohexane (0.0068 mL, 0.057 mmol), cesium carbonate (90 mg, 0.28 mol) and N-methylpyrrolidinone (0.5 mL) was heated in an Initiator™ Microwave Synthesizer at 180° C. for 6 hours. The cooled mixture was partitioned between ethyl acetate and water. The organic phase was washed with water, then brine, dried (K₂CO₃) and evaporated. The residue was dissolved in methanol/water/2N HCl (3:30:3 mL) and then loaded onto a 10 g tC18 Sep-Pak® cartridge and eluted with a gradient up to methanol/water/2N HCl (35:65:0.5). Evaporation gave the title compound as a buff solid. MS (ES+ve): [M+H]⁺ at m/z 346 (C₂₁H₂₃N₅ requires [M+H]⁺ at m/z 346).

### EXAMPLE 21

### N-[(2,6-Dimethylphenyl)methyl]-2,3-dimethyl-6-(2-methyl-1H-imidazol-1-yl)imidazo[1,2-a]pyridin-8-amine hydrochloride

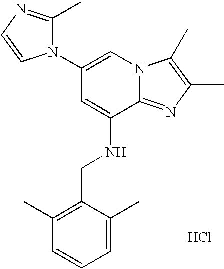

A mixture of 6-bromo-N-[(2,6-dimethylphenyl)methyl]-2,3-dimethylimidazo[1,2-a]pyridin-8-amine (116 mg, 0.325 mmol; WO 98/37080), 2-methylimidazole (264 mg, 3.22 mmol), copper(I) iodide (6.3 mg, 0.033 mmol), trans-1,2-diaminocyclohexane (0.0158 mL, 0.132 mmol), cesium carbonate (209 mg, 0.65 mol) and N-methylpyrrolidinone (1 mL) was heated in an Initiator™ Microwave Synthesizer at 180° C. for 8 hours. The cooled mixture was partitioned between ethyl acetate and water. The organic phase was washed with water, then evaporated. The residue was dissolved in methanol/water/2N HCl (3:30:3 mL) and then loaded onto a 10 g tC18 Sep-Pak® cartridge and eluted with a gradient up to methanol/water/2N HCl (35:65:0.5). Evaporation gave the title compound as a buff solid. MS (ES+ve): [M+H]⁺ at m/z 360 (C₂₂H₂₅N₅ requires [M+H]⁺ at m/z 360).

### EXAMPLES 22 AND 23

### N-[(2,6-Dimethylphenyl)methyl]-2,3-dimethyl-6-(5-methyl-1H-imidazol-1-yl)imidazo[1,2-a]pyridin-8-amine hydrochloride and N-[(2,6-dimethylphenyl)methyl-2,3-dimethyl-6-(4-methyl-1H-imidazol-1-yl)imidazo[1,2-a]pyridin-8-amine hydrochloride

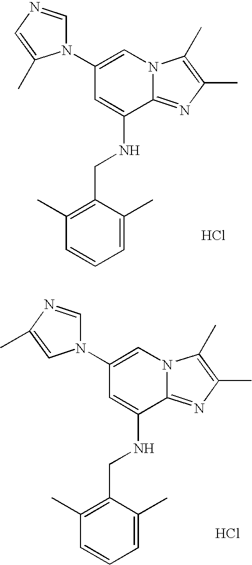

A mixture of 6-bromo-N-[(2,6-dimethylphenyl)methyl]-2,3-dimethylimidazo[1,2-a]pyridin-8-amine (100 mg, 0.279 mmol; WO 98/37080), 4(5)-methylimidazole (228 mg, 2.78 mmol), copper(I) iodide (5.4 mg, 0.028 mmol), trans-1,2-diaminocyclohexane (0.0136 mL, 0.114 mmol), cesium carbonate (180 mg, 0.56 mol) and N-methylpyrrolidinone (1 mL) was heated in an Initiator™ Microwave Synthesizer at 180° C. for 10 hours. The cooled mixture was partitioned between ethyl acetate and water. The organic phase was washed with water, and then evaporated. The residue was dissolved in methanol/water/2N HCl (3:30:3 mL) and then loaded onto a 10 g tC18 Sep-Pak® cartridge and eluted with a gradient up to methanol/water/2N HCl (35:65:0.5). Fractions containing the first component were rechromatographed to give N-[(2,6-dimethylphenyl)methyl]-2,3-dimethyl-6-(5-methyl-1H-imidazol-1-yl)imidazo[1,2-a]pyridin-8-amine hydrochloride (12.9 mg): MS (ES+ve): [M+H]⁺ at m/z 360 (C₂₂H₂₅N₅ requires [M+H]⁺ at m/z 360). Fractions containing the second components from each cartridge were combined and evaporated to give N-[(2,6-dimethylphenyl)methyl]-2,3-dimethyl-6-(4-methyl-1H-imidazol-1-yl)imidazo[1,2-a]pyridin-8-amine hydrochloride (54 mg): MS (ES+ve): [M+H]⁺ at m/z 360 (C₂₂H₂₅N₅ requires [M+H]⁺ at m/z 360).

### EXAMPLE 24

### 3-(8-{[(2,6-Dimethylphenyl)methyl]amino}-2,3-dimethylimidazo[1,2-a]pyridin-6-yl)-2(1H)-pyridinone hydrochloride

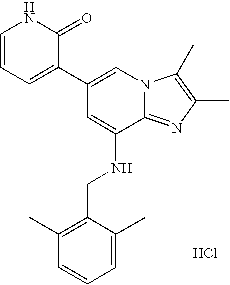

To a solution of N-[(2,6-dimethylphenyl)methyl]-2,3-dimethyl-6-[2-(methyloxy)-3-pyridinyl]imidazo[1,2-a]pyridin-8-amine (66 mg, 0.171 mmol; Example 18) in acetonitrile (2 mL) was added sodium iodide (64 mg, 0.427 mmol) and chlorotrimethylsilane (0.216 mL, 1.69 mmol). The mixture was stirred at room temperature for 1 hour, then 45° C. for 30 minutes, then 60° C. for 1 hour. After cooling 20% ammonium hydroxide solution (1 mL) was added and the mixture was stirred at room temperature for 10 minutes, and then evaporated to dryness. The residue was dissolved in methanol/water/2N HCl (6:15:0.5 mL) and then loaded onto a 10 g tC18 Sep-Pak® cartridge and eluted with a gradient up to methanol/water/2N HCl (80:20:0.5) to give the title compound as a buff solid. MS (ES+ve): [M+H]⁺ at m/z 371 (C₂₃H₂₄N₄O requires [M+H]⁺ at m/z 371).

### EXAMPLE 25a

### 1-(8-{[(2,6-Dimethylphenyl)methyl]amino}-2,3-dimethylimidazo[1,2-a]pyridin-6-yl)-2(1H)-pyridinone hydrochloride

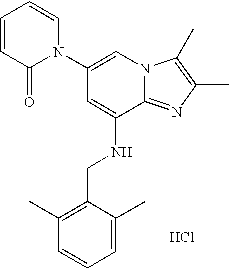

A mixture of 6-bromo-N-[(2,6-dimethylphenyl)methyl]-2,3-dimethylimidazo[1,2-a]pyridin-8-amine (300 mg, 0.837 mmol; WO 98/37080), 2-hydroxypyridine (159 mg, 1.67 mmol), copper(I) iodide (32 mg, 0.17 mmol), potassium carbonate (231 mg, 1.67 mol) and dimethylformamide (4 mL) was heated in an Initiator™ Microwave Synthesizer at 150° C. for 15 hours. The cooled mixture was partitioned between ethyl acetate/water and ammonia and extracted once more with ethyl acetate. The organic phase was washed with water, then brine, dried (MgSO₄) and evaporated. The residue was purified by chromatography on silica gel (ethyl acetate/methanol). The product was dissolved in methanol (5 mL), water (2 mL) and 2N HCl (1 mL) were added. After stirring for 5 minutes, the solution was evaporated to dryness, methanol added and evaporated a second time to give the title compound as a foam. MS (ES+ve): [M+H]⁺ at m/z 373 (C₂₃H₂₄N₄O requires [M+H]⁺ at m/z 373).

### EXAMPLE 25b

### 1-(8-{[(2,6-Dimethylphenyl)methyl]amino}-2,3-dimethylimidazo[1,2-a]pyridin-6-yl)-2(1H)-pyridinone

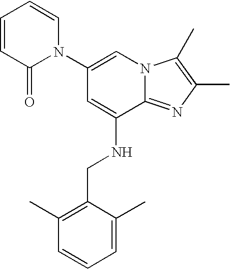

Alternatively a mixture of 6-bromo-N-[(2,6-dimethylphenyl)methyl]-2,3-dimethylimidazo[1,2-a]pyridin-8-amine (100 g, 279 mmol; WO 98/37080), 2-hydroxypyridine (80 g, 837 mmol), copper(I) iodide (16 g, 84 mmol), cesium carbonate (190 g, 586 mmol), N.N′-dimethyl-1,2-ethanediamine (8.9 mL, 84 mmol) in NMP (200 mL) was heated at 160° C. for 5 h. The mixture was allowed to cool to room temperature, diluted with dichloromethane (1 litre), stirred for 30 mins, then filtered through Celite. The filtrate was washed with water (2 Litres; layers allowed to separate overnight), followed by further washings with water (9×1 Litre). After the final wash a layer of emulsion was present. This was separated from the DCM and filtered through Celite. The pad was washed with DCM (300 mls) and the filtrate separated. The DCM layer from this was added to the original DCM portion and dried (Na₂SO₄). The solvent was evaporated to give a dark gum which was triturated under diethyl ether (300 mL) and filtered. This was washed with diethylether and dried in a vacuum oven at 40° C. overnight. The crude solid was dissolved in dichloromethane (150 mL) and purified by column chromatography on silica gel. Elution with ethyl acetate/2M NH₃ in methanol 200:1, evaporation and further trituration with diethylether followed by filtration and further drying in a vacuum oven at 40° C. for 2 days gave the title compound as a buff-coloured solid. MS (ES+ve): [M+H]⁺ at m/z 373 (C₂₃H₂₄N₄O requires [M+H]⁺ at m/z 373).

Proton NMR (CDCl₃; 400 MHz) δ7.45 (1H (m) Ar—H), δ7.35(1H,(d) Ar—H), δ 7.12 (1H, (m) Ar—H), δ7.04 (2H, (m) Ar—H), δ6.70 (1H, (dd) Ar—H), δ6.30 (1H, (t) Ar—H), δ6.17 (1H, (d) Ar—H), δ4.97 (1H (broad t), NH), δ4.33 (2H, (d) CH₂), δ2.37 (6H, (s) CH₃), δ2.34 (6H, (s) CH₃).

### EXAMPLE 25c

### 1-(8-{1[(2,6-Dimethylphenyl)methyl]amino}-2,3-dimethylimidazo[1,2-a]pyridin-6-yl)-2(1H)-pyridinone hydrochloride

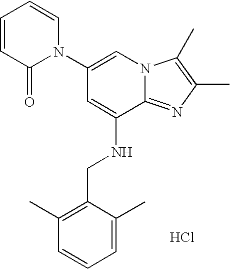

Alternatively, a mixture of 6-bromo-N-[(2,6-dimethylphenyl)methyl]-2,3-dimethylimidazo[1,2-a]pyridin-8-amine (5 g, 0.0140 mol; WO 98/37080), 2-pyridone (4 g, 0.042 mol), copper(I) iodide (0.795 g, 4.17 mmol), cesium carbonate (9.55 g, 0.0293 mol), N.N′-dimethyl-1,2-ethanediamine (365 mg, 4.17 mmol) in N-methylpyrrolidinone (100 mL) was heated at 160° C. for 18 h. The mixture was allowed to cool to room temperature, diluted with ethyl acetate (400 mL) then filtered through Celite. The filtrate was washed (5×300 mL) with brine/water 1:1 (an initial filtration through Celite was required during the first wash). The organic phase was dried (Na₂SO₄) and evaporated. The residue was purified by column chromatography on silica gel. Elution with ethyl acetate gave an almost colourless foam. The HCl salt was prepared from the above (3.57 g) by the addition of 1M HCl in diethylether (25 mL) to a solution of free base in ether (80 mL) containing methanol (20 mL). The solvent was evaporated to give a buff-coloured solid. MS (ES+ve): [M+H]⁺ at m/z 373 (C₂₃H₂₄N₄O requires [M+H]⁺ at m/z 373).

### EXAMPLE 26

### 1-(8-{[(2,6-Dimethylphenyl)methyl]amino}-2,3-dimethylimidazo[1,2-a]pyridin-6-yl)-4(1H)-pyridinone hydrochloride

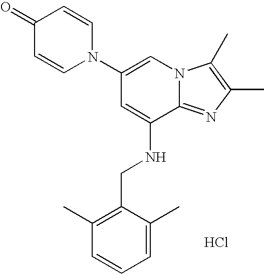

A mixture of 6-bromo-N-[(2,6-dimethylphenyl)methyl]-2,3-dimethylimidazo[1,2-a]pyridin-8-amine (200 mg, 0.56 mmol; WO 98/37080), 4-hydroxypyridine (106 mg, 1.12 mmol), copper(I) iodide (21 mg, 0.11 mmol), potassium carbonate (154 mg, 1.12 mol) and dimethylformamide (3 mL) was heated in an Initiator™ Microwave Synthesizer at 150° C. for 6 hours. The cooled mixture was partitioned between ethyl acetate and water. The organic phase was washed with water, then brine, dried (MgSO₄) and evaporated. The residue was purified by chromatography on silica gel (ethyl acetate/methanol). The product was dissolved in methanol/water/2N HCl (4.5:25:0.5 mL) and then loaded onto a 10 g tC18 Sep-Pak® cartridge and eluted with a gradient up to methanol/water/2N HCl (40:60:0.5) to give the title compound as a buff solid. MS (ES+ve): [M+H]⁺ at m/z 373 (C₂₃H₂₄N₄O requires [M+H]⁺ at m/z 373).

### EXAMPLE 27

### 1-(8-{[(2,6-Dimethylphenyl)methyl]amino}-2,3-dimethylimidazo[1,2-a]pyridin-6-yl)-4-methyl-2(1H)-pyridinone hydrochloride

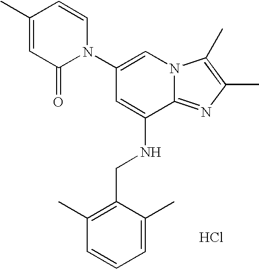

A mixture of 6-bromo-N-[(2,6-dimethylphenyl)methyl]-2,3-dimethylimidazo[1,2-a]pyridin-8-amine (100 mg, 0.28 mmol; WO 98/37080), 2-hydroxy-4-methylpyridine (61 mg, 0.56 mmol), copper(I) iodide (10.6 mg, 0.056 mmol), potassium carbonate (77 mg, 0.56 mol) and dimethylformamide (2 mL) was heated in an Initiator™ Microwave Synthesizer at 150° C. for 12 hours. Methanol (10 mL) and water (1 mL) were added to the cooled reaction mixture, which was then applied to an Isolute® SCX cartridge. Elution with methanol, then 1M NH₃ in methanol gave, after evaporation, the product which was further purified by chromatography on silica gel (ethyl acetate/methanol). The product was dissolved in methanol (5 mL), water (1 mL) and 2N HCl (0.2 mL) were added. After stirring for 5 minutes, the solution was evaporated to give the title compound as a buff solid. MS (ES+ve): [M+H]⁺ at m/z 387 (C₂₄H₂₆N₄O requires [M+H]⁺ at m/z 387).

### EXAMPLE 28

### 5-Chloro-1-(8-{[(2,6-dimethylphenyl)methyl]amino}-2,3-dimethylimidazo[1,2-a]pyridin-6-yl)-2(1H)-pyridinone hydrochloride

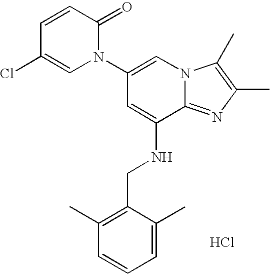

A mixture of 6-bromo-N-[(2,6-dimethylphenyl)methyl]-2,3-dimethylimidazo[1,2-a]pyridin-8-amine (100 mg, 0.28 mmol; WO 98/37080), 5-chloro-2-hydroxypyridine (73 mg, 0.56 mmol), copper(I) iodide (10.6 mg, 0.056 mmol), potassium carbonate (77 mg, 0.56 mol) and N-methylpyrrolidinone (2 ml) was heated in an Initiator™ Microwave Synthesizer at 195° C. for 5 hours. Methanol (10 ml) and water (1 ml) were added to the cooled reaction mixture, which was then applied to an Isolute® SCX cartridge. Elution with methanol, then 1M NH₃ in methanol gave, after evaporation, the product which was further purified by chromatography on silica gel (ethyl acetate/methanol). The product was further purified by chromatography on a 10 g tC18 Sep-Pak® cartridge, elution with a gradient up to methanol/water/2N HCl (40:60:0.5) gave the title compound as a buff solid. MS (ES+ve): [M+H]⁺ at m/z 407 (C₂₃H₂₃³⁵Cl N₄O requires [M+H]⁺ at m/z 407).

### EXAMPLE 29

### N-[(2,6-Dimethylphenyl)methyl]-6-(4-fluorophenyl)-2,3-dimethylimidazo[1,2-a]pyridin-8-amine

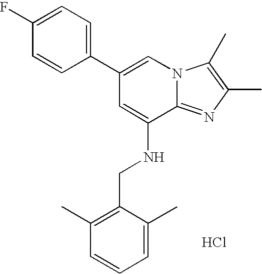

A mixture of 6-bromo-N-[(2,6-dimethylphenyl)methyl]-2,3-dimethylimidazo[1,2-a]pyridin-8-amine (94 mg, 0.263 mmol; WO 98/37080), 4-fluorophenylboronic acid (110 mg, 0.786 mmol), tetrakis(triphenylphosphine)palladium (18 mg, 0.016 mmol), potassium tert-butoxide (321 mg, 2.63 mmol), 1,2-dimethoxyethane (3 mL) and water (1 mL) was heated in an Initiator™ Microwave Synthesizer at 100° C. for 1 hour. The cooled reaction mixture was loaded onto an Isolute® SCX cartridge, elution with methanol, then 1M NH₃ in methanol gave the product which was further purified by MDAP. This product was dissolved in dichloromethane (2 mL), and then 1M HCl in diethyl ether (0.8 mL) was added. After 2 hours the solvents were evaporated to give the title compound; MS (ES+ve): [M+H]⁺ at m/z 373 (C₂₄H₂₄FN₃ requires [M+H]⁺ at m/z 373).

### EXAMPLE 30

### 5-(8-{[(2,6-Dimethylphenyl)methyl]amino}-2,3-dimethylimidazo[1,2-a]pyridin-6-yl)-2-furancarboxamide hydrochloride

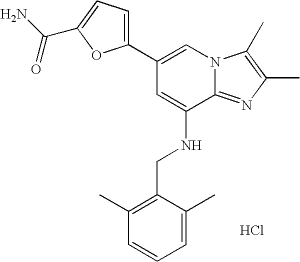

To a mixture of 5-(8-{[(2,6-dimethylphenyl)methyl]amino}-2,3-dimethylimidazo[1,2-a]pyridin-6-yl)-2-furancarboxylic acid (42 mg, 0.108 mmol; Description 13), 1-hydroxybenzotriazole ammonium salt (25 mg, 0.162 mmol; Description 14) and N-ethylmorpholine (25 mg, 0.216 mmol) in dimethylformamide (5 mL), was added 1-(3-dimethylaminopropyl)-3-ethylcarbodiimide hydrochloride (25 mg, 0.13 mmol). The mixture was stirred at room temperature for 16 hours, and then partitioned between ethyl acetate and water. The organic phase was concentrated and the residue was purified by chromatography on silica gel (dichloromethane/methanol) to give the product. This product was dissolved in dichloromethane (2 mL), and then 1M HCl in diethyl ether (1 mL) was added. After 30 minutes the solvents were evaporated to give the title compound. MS (ES+ve): [M+H]⁺ at m/z 389 (C₂₃H₂₄N₄O₂ requires [M+H]⁺ at m/z 389).

### EXAMPLE 31

### 6-(8-{[(2,6-Dimethylphenyl)methyl]amino}-2,3-dimethylimidazo[1,2-a]pyridin-6-yl)-2-pyridinecarboxamide hydrochloride

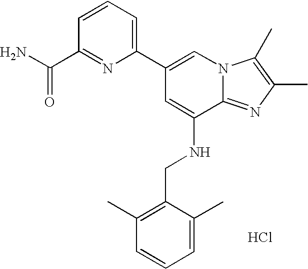

To a solution of methyl 6-(8-{[(2,6-dimethylphenyl)methyl]amino}-2,3-dimethylimidazo[1,2-a]pyridin-6-yl)-2-pyridinecarboxylate (92 mg, 0.22 mmol; Description 15) in tetrahydrofuran (5 mL) was added an aqueous solution of lithium hydroxide (0.5N, 5 mL). The mixture was stirred at room temperature for 16 hours. The mixture was acidified by the addition of 5N HCl, and then evaporated to give a mixture of 6-(8-{[(2,6-dimethylphenyl)methyl]amino}-2,3-dimethylimidazo[1,2-a]pyridin-6-yl)-2-pyridinecarboxylic acid and lithium chloride. To this mixture were added dimethylformamide (3 mL), 1-hydroxybenzotriazole ammonium salt (43 mg, 0.28 mmol; Description 14), N-ethylmorpholine (43 mg, 0.37 mmol) and 1-(3-dimethylaminopropyl)-3-ethylcarbodiimide hydrochloride (43 mg, 0.22 mmol). The mixture was stirred at room temperature for 16 hours. The reaction mixture was applied to an Isolute® PE-AX cartridge and eluted first with methanol, then with 5% formic acid in methanol (the latter to recover unreacted 6-(8-{[(2,6-dimethylphenyl)methyl]amino}-2,3-dimethylimidazo[1,2-a]pyridin-6-yl)-2-pyridinecarboxylic acid. The methanol washings were concentrated and the residue was purified by chromatography on silica gel (ethyl acetate/hexane) to give the product. This product was dissolved in dichloromethane (2 mL), and then 1M HCl in diethyl ether (1 mL) was added. After 30 minutes the solvents were evaporated to give the title compound. MS (ES+ve): [M+H]⁺at m/z 400 (C₂₄H₂₅N₅O requires [M+H]⁺ at m/z 400).

### EXAMPLE 32

### 1-(8-{[(2,6-Dimethylphenyl)methyl]amino}-2,3-dimethylimidazo[1,2-a]pyridin-6-yl)-1H-pyrazole-4-carboxamide hydrochloride

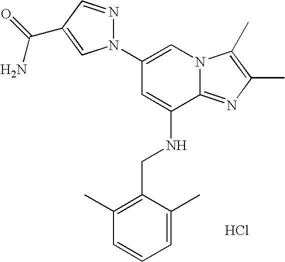

A mixture of 6-bromo-N-[(2,6-dimethylphenyl)methyl]-2,3-dimethylimidazo[1,2-a]pyridin-8-amine (74 mg, 0.207 mmol; WO 98/37080), 1H-Pyrazole-4-carboxamide (Description 16) (46 mg, 0.414 mmol), copper(I) iodide (12 mg, 0.062 mmol), trans-1,2-diaminocyclohexane (0.0074 mL, 0.062 mmol), potassium phosphate (158 mg, 0.746 mol) and dioxane (5 mL) was heated under reflux for 5 days. The cooled mixture was partitioned between ethyl acetate and water. The organic phase was concentrated and the residue purified by MDAP. The product was dissolved in dichloromethane (2 mL), and then 1M HCl in diethyl ether (1 mL) was added. After 30 minutes the solvents were evaporated to give the title compound. MS (ES+ve): [M+H]⁺ at m/z 389 (C₂₂H₂₄N₆O requires [M+H]⁺ at m/z 389).

### EXAMPLE 33

### N-[(2,6-Dimethylphenyl)methyl]-2,3-dimethyl-6-(1H-1,2,3-triazol-4-yl)imidazo[1,2-a]pyridin-8-amine hydrochloride

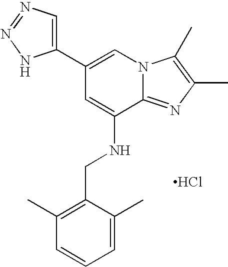

Trimethylsilyl azide (158 mg, 1.37 mmol) was added to a solution of N-[(2,6-dimethylphenyl)methyl]-6-ethynyl-2,3-dimethylimidazo[1,2-a]pyridin-8-amine (278 mg, 0.92 mmol; Description 17) in dimethylformamide/methanol (9:1, 5 mL) containing copper(I) iodide (4 mg, 0.02 mmol). The reaction mixture was stirred at 100° C. for 16 hours. The cooled mixture was loaded onto an Isolute(g) SCX cartridge, eluting with methanol, then 2M NH₃ in methanol. Fractions containing the product were concentrated and subjected to MDAP purification. The product was dissolved in methanol (5 mL) and treated with 1M HCl in diethyl ether (1 mL). After stirring for 10 minutes, the solvents were evaporated to yield the title compound. MS (ES+ve): [M+H]⁺ at m/z 347 (C₂₀H₂₂N₆ requires [M+H]⁺ at m/z 347).

### EXAMPLE 34

### 6-(5-Amino-2-pyridinyl)-N-[(2,6-dimethylphenyl)methyl]-2,3-dimethylimidazo[1,2-a]pyridin-8-amine

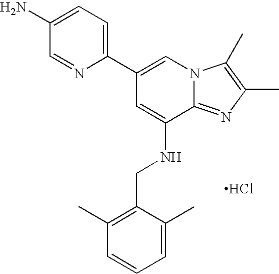

To a solution of N-[(2,6-dimethylphenyl)methyl]-2,3-dimethyl-6-(5-nitro-2-pyridinyl)imidazo[1,2-a]pyridin-8-amine (50 mg, 0.12 mmol; Description 18) in ethanol (2.3 mL) and ethyl acetate (2.3 mL) was added tin(II) chloride hydrate (140 mg, 0.62 mmol). The reaction mixture was heated at reflux for 24 hours, cooled and then treated with dilute aqueous sodium hydrogen carbonate solution. The resulting precipitate was removed by filtration through celite which was washed with ethyl acetate. The filtrate was washed with water and brine, dried (MgSO₄) and evaporated to yield the free base of the title compound. This was dissolved in dichloromethane (2 mL) and treated with 1M HCl in diethyl ether (1 mL). After stirring for 10 minutes, the solvents were evaporated to yield the title compound. MS (ES+ve): [M+H]⁺ at m/z 372 (C₂₃H₂₅N₅ requires [M+H]⁺ at m/z 372).

The following compounds were prepared by reaction of 6-bromo-N-[(2,6-dimethylphenyl)methyl]-2,3-dimethylimidazo[1,2-a]pyridin-8-amine (WO 98/37080) with the appropriate boronic acid or boronic ester (as indicated) in an analogous manner to that described for example 11, altering reaction temperatures, times, solvents and drying agents as appropriate:

| | | | Characterisation |
|---|---|---|---|
| | | | MS(ES + ve) |
| Example | | Boronic acid/ | m/z[M + H]⁺ |
| No. | Structure/Name of product | ester | observed. |
| 35 | 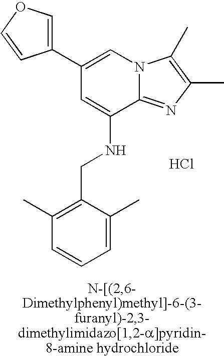 | 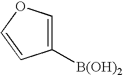 | 346 |
| 36 | 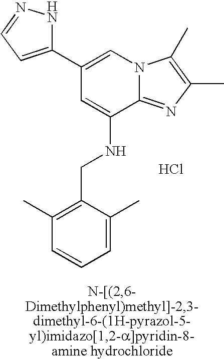 | 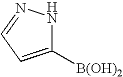 | 346 |
| 37 | 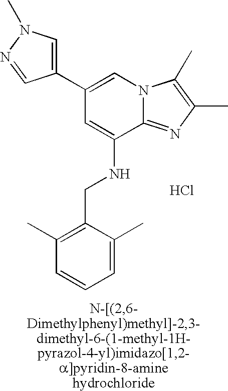 | 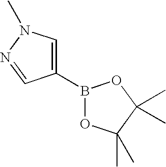 | 360 |
| 38 | 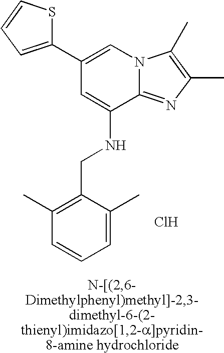 | 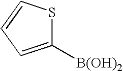 | 362 |

### EXAMPLE 39a

### N-[(2,6-Dimethylphenyl)methyl]-2,3-dimethyl-6-(1H-1,2,4-triazol-1-yl)imidazo[1,2-a]pyridin-8-amine

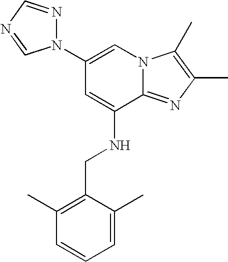

6-bromo-N-[(2,6-dimethylphenyl)methyl]-2,3-dimethylimidazo[1,2-a]pyridin-8-amine (50 g, 139 mmol; WO 98/37080), was added portionwise to a mixture of 1H-1,2,4-triazole (29 g, 420 mmol), copper(I) iodide (7.95 g, 42 mmol), cesium carbonate (91 g, 279 mol) and N.N′-dimethyl-1,2-ethanediamine (3.68 g, 42 mmol) in NMP (250 mL) at 160-165° C. The mixture was stirred at 168° C. for 8 h, cooled to room temperature, diluted with dichloromethane (1 litre) and filtered through Hyflo. The filtrate was washed with water (6×500 ml) and dried (Na₂SO₄). The solvent was evaporated to give a dark oil which was dissolved in DCM (50 ml) and purified by column chromatography on silica gel. Elution with hexane/ethyl acetate 1:1 gave a pale yellow solid which was triturated under pentane (25 ml) and filtered to give the title compound as a buff-coloured solid. MS (ES+ve): [M+H]⁺ at m/z 347 (C₂₀H₂₂N₆ requires [M+H]⁺ at m/z 347).

Proton NMR (CDCl₃; 400 MHz) 68.56. (1H (s) Ar—H), δ8.15(1H,(s) Ar—H), δ 7.67 (1H, (d) Ar—H), δ7.15 (1H, (m) Ar—H), δ7.06 (2H, (m) Ar—H), δ6.43 (1H, (d) Ar—H), δ5.09 (1H (broad t), NH), δ4.41 (2H, (d) CH₂); δ2.40 (3H, (s) CH₃), δ2.39 (6H, (s) 2×CH₃), δ2.36 (3H, (s) CH₃).

### EXAMPLE 39b

### N-[(2,6-Dimethylphenyl)methyl]-2,3-dimethyl-6-(1H-1,2,4-triazol-1-yl)imidazo[1,2-a]pyridin-8-amine hydrochloride

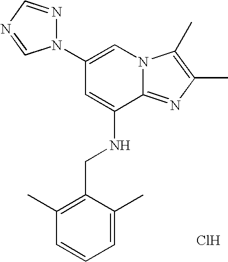

Alternatively 6-bromo-N-[(2,6-dimethylphenyl)methyl]-2,3-dimethylimidazo[1,2-a]pyridin-8-amine (2 g, 5.58 mmol; WO98/37080), was added to a mixture of 1H-1,2,4-triazole (1.16 g, 16.76 mmol), copper(I) iodide (319 mg, 1.67 mmol), cesium carbonate (3.82 g, 11.7 mol) and N.N′-dimethyl-1,2-ethanediamine (147 mg, 1.67 mmol) in N-methylpyrrolidinone (50 mL). The mixture was stirred at 180° C. for 48 h, cooled to room temperature, diluted with ethyl acetate (100 mL) and filtered. The filtrate was washed with water/brine (3×100 mL) and the organic phase dried (Na₂SO₄). The solvent was filtered and evaporated. The residue was purified by column chromatography on silica gel. Elution with 25%-75% ethyl acetate in hexane gave a pale brown solid.

The HCl salt was prepared by dissolving the free base (998 mg) in DCM (50 mL) and adding 1M HCl in ethylacetate (10 mL). This was then evaporated and triturated under ethyl acetate (50 mL) to give a buff coloured solid (1.147 g) MS (ES+ve): [M+H]⁺ at m/z 347 (C₂₀H₂₂N₆ requires [M+H]⁺ at m/z 347).

The following compounds were prepared from 6-bromo-N-[(2,6-dimethylphenyl)methyl]-2,3-dimethylimidazo[1,2-a]pyridin-8-amine (WO98/37080), by reacting with the appropriate heteroaryl reagent in an analogous manner to that described for example 28, altering reaction temperatures, times, solvents and drying agents as appropriate:

| | | | Characterisation |
|---|---|---|---|
| | | | MS(ES + ve) |
| Example | | Heteroaryl | m/z[M + H]⁺ |
| No. | Structure/Name of product | reagent | observed |
| 39c | 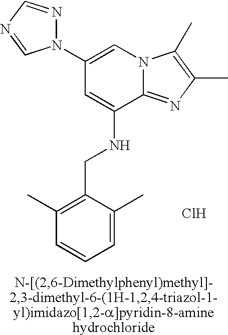 | 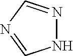 | 347 |
| 40 | 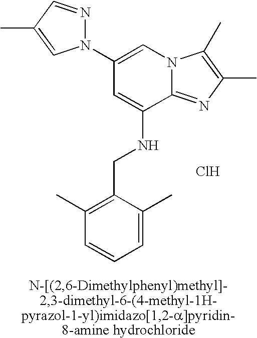 | 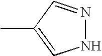 | 360 |
| 41 | 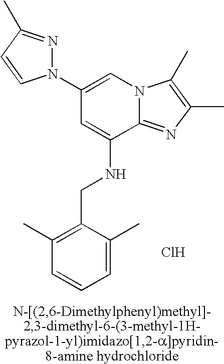 | 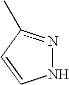 | 360 |
| 42 | 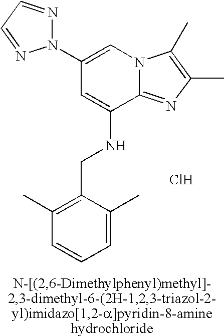 | 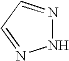 | 347 |

### EXAMPLE 43

### N-[(2-Chloro-4-fluorophenyl)methyl]-2,3-dimethyl-6-(1H-pyrazol-1-yl)imidazo[1,2-a]pyridin-8-amine

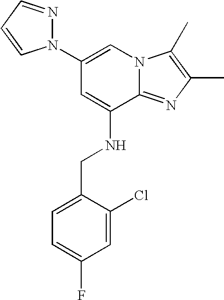

A mixture of 2,3-dimethyl-6-(1H-pyrazol-1-yl)imidazo[1,2-a]pyridin-8-amine (Description 20, 100 mg, 0.44 mmol), 1-(bromomethyl)-2-chloro-4-fluorobenzene (295 mg, 1.32 mmol), sodium carbonate (148 mg, 1.40 mmol) and dimethylformamide (5 mL) were stirred at 60° C. for 16 hours. The mixture was partitioned between ethyl acetate and water. The organic phase was washed with water, dried over magnesium sulphate and evaporated. The product was purified by chromatography on silica gel and eluted with 0-50% ethyl acetate in hexane. The solvent was then evaporated to give the title compound. MS (ES+ve): [M+H]⁺ at m/z 370/372 (C₁₉H₁₇FN₅Cl requires [M+H]⁺ at m/z 370/372).

The following compounds were prepared from 2,3-dimethyl-6-(1H-pyrazol-1-yl)imidazo[1,2-a]pyridin-8-amine (Description 20) by reaction with the appropriate alkyl halide in a manner analogous to that of example 43, altering reaction temperatures, times, solvents and drying agents as appropriate:

| | | | Characterisation |
|---|---|---|---|
| | | | MS(ES + ve) |
| Example | | | m/z[M + H]⁺ |
| No: | Structure/Name of product | Alkyl halide | observed |
| 44 | 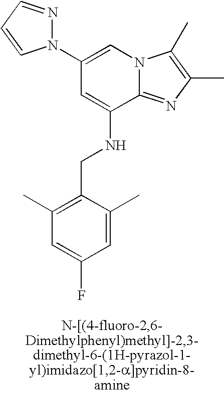 | 2-(bromomethyl)-5-fluoro-1,3-dimethylbenzene | 364 |
| 45 | 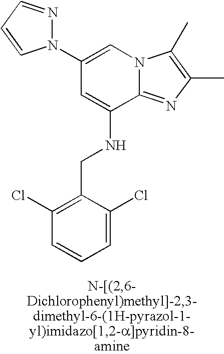 | 2-(bromomethyl)-1,3-dichlorobenzene | 386/388/390 |
| 46 | 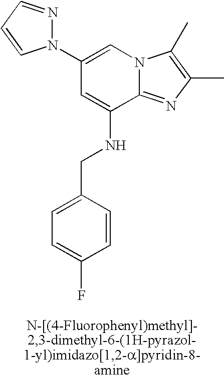 | 1-(bromomethyl)-4-fluorobenzene | 336 |
| 47 | 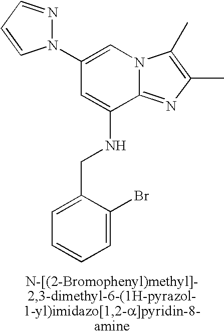 | 1-bromo-2-(bromomethyl)benzene | 396/398 |
| 48 | 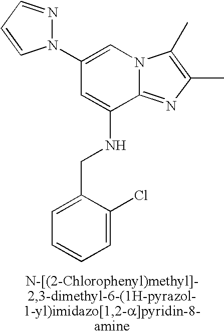 | 1-(bromomethyl)-2-chlorobenzene | 352/254 |
| 49 | 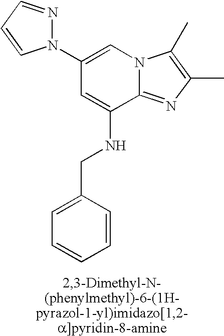 | (bromomethyl)benzene | 318 |
| 50 | 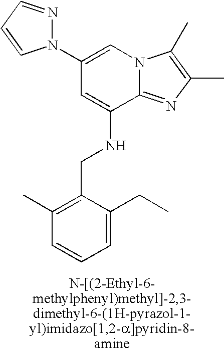 | 2-(bromomethyl)-1-ethyl-3-methylbenzene(Description 6) | 360 |
| 51 | 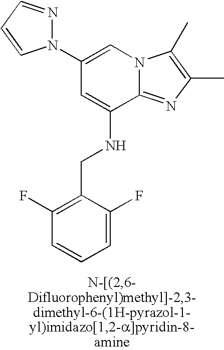 | 2-(bromomethyl)-1,3-difluorobenzene | 354 |

### EXAMPLE 52

### 1-(8-{[(2,6-Difluorophenyl)methyl]amino}-2,3-dimethylimidazo[1,2-a]pyridin-6-yl)-2(1H)-pyridinone hydrochloride

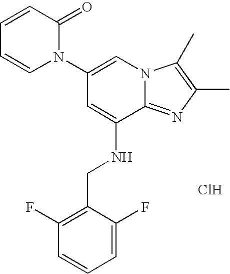

A mixture of 1-(8-amino-2,3-dimethylimidazo[1,2-a]pyridin-6-yl)-2(1H)-pyridinone (Description 19, 100 mg, 0.39 mmol), 2-(bromomethyl)-1,3-difluorobenzene (161 mg, 0.78 mmol), sodium carbonate (120 mg, 1.25 mmol) and dimethylformamide (5 mL) were stirred at room temperature for 48 hours. The mixture was partitioned between ethyl acetate and water. The organic phase was washed with water, dried over magnesium sulphate and evaporated. The product was purified by MDAP. Fractions containing the product were combined and evaporated. The product was re-dissolved into ethyl acetate, washed with sodium bicarbonate and water and all solvents evaporated to give the free base of the title compound. The purified product was then treated with 1M hydrochloric acid in diethyl ether (1 mL) and mixed thoroughly, after which all solvents were evaporated to give the title compound. MS (ES+ve): [M+H]⁺ at m/z 381 (C₂₁H₁₈F₂N₄O requires [M+H]⁺ at m/z 381).

The following compounds were prepared from 1-(8-amino-2,3-dimethylimidazo[1,2-a]pyridin-6-yl)-2(1H)-pyridinone (Description 19) by reaction with the appropriate alkyl halide in a manner analogous to that of example 52, altering reaction temperatures, times, solvents and drying agents as appropriate. HCl salts were prepared as indicated, in an appropriate manner, for examples 53 to 56.

| | | | Characterisation |
|---|---|---|---|
| | | | MS(ES + ve) |
| | | | m/z[M + H]⁺ |
| Example | Structure/Compound Name | Alkyl halide | observed |
| 53 | 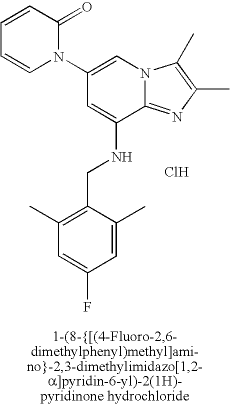 | 2-(Bromomethyl)-5-fluoro-1,3-dimethylbenzene | 391 |
| 54 | 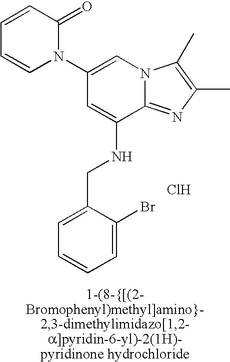 | 1-Bromo-2-(bromomethyl)ben-zene | 423 |
| 55 | 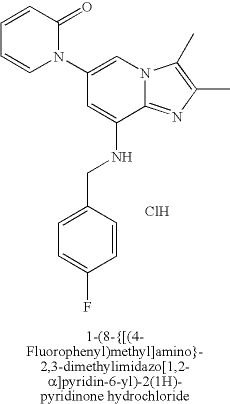 | 1-(Bromomethyl)-4-fluorobenzene | 363 |
| 56 | 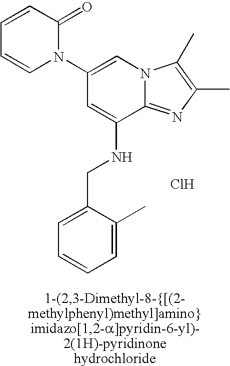 | 1-(Bromomethyl)-2-methylbenzene | 359 |
| 57 | 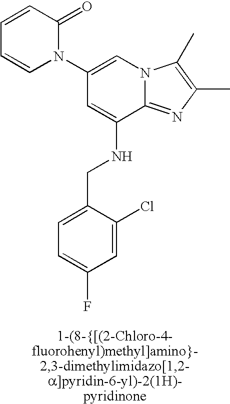 | 1-(Bromomethyl)-2-chloro-4-fluorobenzene | 397/399 |
| 58 | 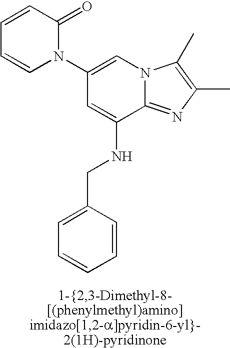 | (Bromomethyl)-benzene | 345 |
| 59 | 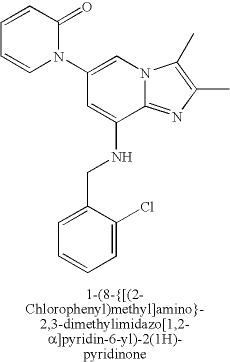 | 1-(Bromomethyl)-2-chlorobenzene | 379/381 |
| 60 | 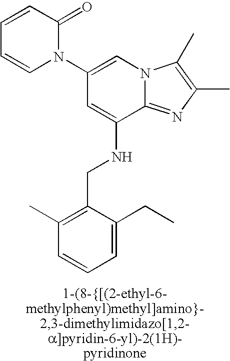 | 2-(bromomethyl)-1-ethyl-3-methylbenzene(Description 6) | m/z 387(C₂₄H₂₆N₄O)requires [M + H]⁺at m/z 387 |

### EXAMPLE 61

### 1-(8-{[(4-Fluorophenyl)methyl]oxy}-2,3-dimethylimidazo[1,2-a]pyridin-6-yl)-2(1H)-pyridinone

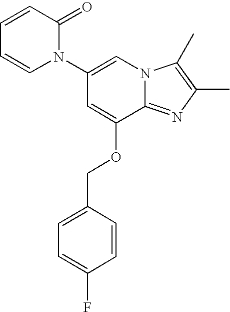

Sodium hydride (9 mg of a 60% dispersion in mineral oil, 0.22 mmol) was added to a solution of 1-(8-hydroxy-2,3-dimethylimidazo[1,2-a]pyridin-6-yl)-2(1H)-pyridinone (50 mg, 0.20 mmol Description 23) in dimethylformamide (3 mL) and the mixture stirred for 90 minutes. 1-(Bromomethyl)-4-fluorobenzene (50 μL, 0.40 mmol) was added and the mixture allowed to react at room temperature overnight. The mixture was applied to an Isolute® SCX cartridge and eluted with methanol followed by 2M NH₃ in methanol. The basic fractions were combined and evaporated under reduced pressure. This residue was purified by column chromatography on silica eluting with 2-98 2M NH₃ in methanol-dichloromethane to afford the title compound MS (ES+ve): [M+H]⁺ at m/z 364 (C₂₁H₁₈FN₃O₂ requires [M+H]⁺ at m/z 364).

### EXAMPLE 62

### 1-{2,3-Dimethyl-8-[(phenylmethyl)oxy]imidazo[1,2-a]pyridin-6-yl}-2(1H)-pyridinone

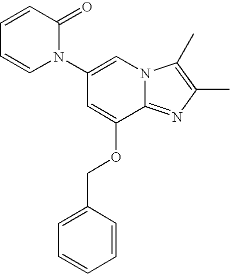

Sodium hydride (9 mg of a 60% dispersion in mineral oil, 0.22 mmol) was added to a solution of 1-(8-hydroxy-2,3-dimethylimidazo[1,2-a]pyridin-6-yl)-2(1H)-pyridinone (50 mg, 0.20 mmol Description 23) in DMF (3 mL) and the mixture stirred for 2 hours. Bromomethylbenzene (26 μl, 0.22 mmol) was added and the mixture allowed to react at room temperature for 3 hours. The mixture was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane to afford the title compound MS (ES+ve): [M+H]⁺ at m/z 346 (C₂₁H₁₉N₃O₂ requires [M+H]⁺ at m/z 346).

Examples 63-66 were prepared using an analogous method to that used to to prepare Example 62 using 1-(8-hydroxy-2,3-dimethylimidazo[1,2-a]pyridin-6-yl)-2(1H)-pyridinone (Description 23), the appropriate alkyl halide shown in the table below and altering reaction temperatures, times, solvents and drying agents as appropriate:

| | | | Characterisation |
|---|---|---|---|
| | | | MS(ES + ve) |
| Example | | | m/z[M + H]⁺ |
| No | Structure/Compound Name | Alkyl Halide | observed |
| 63 | 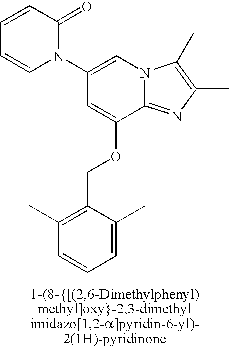 | 2-(Chloromethyl)-1,3-dimethylbenzene | 374 |
| 64 | 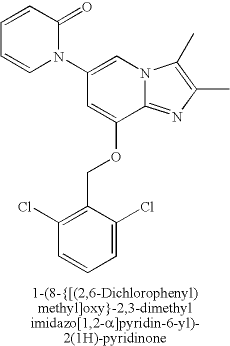 | 2-(Bromomethyl)-1,3-dichlorobenzene | 414 |
| 65 | 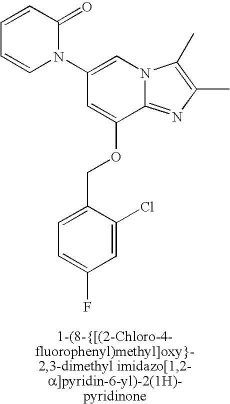 | 1-(Bromomethyl)-2-chloro-4-fluorobenzene | 398 |
| 66 | 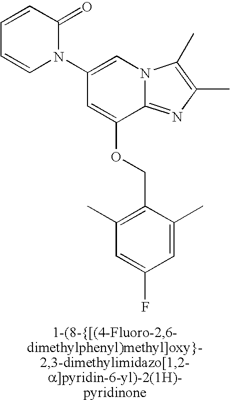 | 2-(Bromomethyl)-5-fluoro-1,3-dimethylbenzene | 392 |

### EXAMPLE 67

### 1-[8-{[(2,6-Dimethylphenyl)methyl]amino}-3-(hydroxymethyl)-2-methylimidazo[1,2-a]pyridin-6-yl]-2(1H)-pyridinone

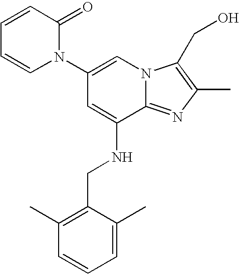

A solution of [8-{[(2,6-dimethylphenyl)methyl]amino}-2-methyl-6-(2-oxo-1(2H)-pyridinyl)imidazo[1,2-a]pyridin-3-yl]-N,N,N-trimethylmethanaminium iodide (50 mg, 0.09 mmol Description 28) in water (1 ml) and tetrahydrofuran (1 ml) was treated with 2M sodium hydroxide solution (1 drop) and heated at reflux for 18 hours. The mixture was allowed to cool and was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane and then by mass directed auto prep to afford the title compound MS (ES+ve): [M+H]⁺ at m/z 389 (C₂₃H₂₄N₄O₂ requires [M+H]⁺ at m/z 389).

### EXAMPLE 68

### [8-{[(2,6-dimethylphenyl)methyl]amino}-2-methyl-6-(1H-1,2,4-triazol-1-yl)imidazo[1,2-a]pyridin-3-yl]methanol

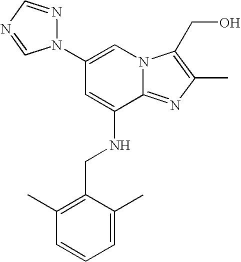

Methyl iodide (0.1 mL) was added to a stirred solution of 3-[(dimethylamino)methyl]-N-[(2,6-dimethylphenyl)methyl]-2-methyl-6-(1H-1,2,4-triazol-1-yl)imidazo[1,2-a]pyridin-8-amine (Description 30; 257 mg, 0.660 mmol) in ethanol (5 mL) and the mixture stirred at room temperature under argon for 16 h resulting in the precipitation of a colourless solid. The solvent was evaporated and the residue triturated under ether (2×5 mL). The solvent was removed by decantation and the residue dried. A portion of this material (113 mg) was heated at 80° C. in a mixture of THF (3 mL), water (0.5 mL) and sodium hydroxide (1M; 3 drops) for 16 h. The cooled mixture was partitioned between ethyl acetate (3×10 ml) and brine (10 ml) and the combined organic extracts were dried (Na₂SO₄). The solvent was evaporated and the residue purified by chromatography on silica. Elution with dichloromethane/methanolic ammonia (2% and increasing the polarity to 10%) gave the title compound as a colourless solid. MS (ES+ve): [M+H]⁺ at m/z 363 (C₂₀H₂₂N₆O requires [M+H]⁺ at m/z 363).

### EXAMPLE 69

### 1-[8-{[(2,6-dimethylphenyl)methyl]amino}-3-(hydroxymethyl)-2-methylimidazo[1,2-a]pyridin-6-yl]-4-methyl-2(1H)-pyridinone hydrochloride

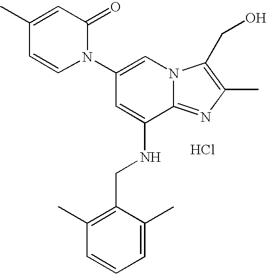

A mixture of [8-{[(2,6-dimethylphenyl)methyl]amino}-2-methyl-6-(4-methyl-2-oxo-1(2H)-pyridinyl)imidazo[1,2-a]pyridin-3-yl]-N,N,N-trimethylmethanaminium iodide (Description 33, 143 mg, 0.25 mmol) and 2M sodium hydroxide (2 drops) was refluxed in tetrahydrofuran (5 ml) and water (1 ml) for 24 hours, allowed to cool, partitioned between ethyl acetate and water. The organic layer was washed with water, brine, dried (MgSO₄), evaporated and the residue purified by chromatography on silica gel (2M methanolic ammonia/dichloromethane) and evaporated. This was redissolved in dichloromethane (DCM) and treated with 1M HCl in diethyl ether (0.05 ml, 0.05 mmol) and evaporated to give the title compound. MS (ES+ve): [M+H]⁺ at m/z 403 (C₂₄H₂₆N₄O₂ requires [M+H]⁺ at m/z 403).

### EXAMPLE 70

### 1-[8-{[(2,6-dimethylphenyl)methyl]oxy}-3-(hydroxymethyl)-2-methylimidazo[1,2-a]pyridin-6-yl]-2(1H)-pyridinone

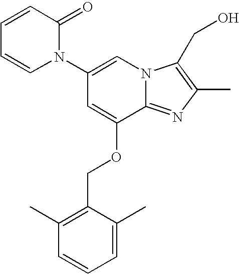

To a solution of 8-{[(2,6-dimethylphenyl)methyl]oxy}-2-methyl-6-(2-oxo-1(2H)-pyridinyl)imidazo[1,2-a]pyridine-3-carbaldehyde (Description 42, 320 mg, 0.8 mmol) in methanol (5 ml) at 0° C. was added sodium borohydride (40 mg, 1.1 mmol) and the resulting mixture was stirred for 30 min at this temperature. Most of the solvent was removed in vacuo and the residue was partitioned between ethyl acetate and a saturated aqueous sodium bicarbonate solution. The two layers were separated and the aqueous phase was extracted with ethyl acetate. The combined organic phases were washed with a saturated aqueous sodium bicarbonate solution, dried (MgSO₄) and concentrated in vacuo. Purification of the residue by flash chromatography on silica gel (dichloromethane/methanol: 99/1 to 95/5) gave the title compound as a white foam. MS (ES+ve): [M+H]⁺ at m/z 390 (C₂₃H₂₁N₃O₃ requires [M+H]⁺ at m/z 390).

### EXAMPLE 71

### [8-{[(2,6-Dimethylphenyl)methyl]oxy}-2-methyl-6-(2-oxo-1(2H)-pyridinyl)imidazo[1,2-a]pyridin-3-yl]acetonitrile

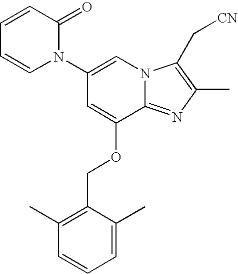

A mixture of [8-{[(2,6-dimethylphenyl)methyl]oxy}-2-methyl-6-(2-oxo-1(2H)-pyridinyl)imidazo[1,2-a]pyridin-3-yl]-N,N,N-trimethylmethanaminium iodide (Description 44, 240 mg, 0.43 mmol) and sodium cyanide (26 mg, 0.52 mmol) in dimethylformamide (4 ml) was heated at 100° C. for 2 hours. The mixture was allowed to cool and was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane to afford the title compound MS (ES+ve): [M+H]⁺ at m/z 399 (C₂₄H₂₂N₄O₂ requires [M+H]⁺ at m/z 399).

### EXAMPLE 72

### 2-(8-{[(2,6-Dimethylphenyl)methyl]amino}-2,3-dimethylimidazo[1,2-a]pyridin-6-yl)-3(2H)-pyridazinone hydrochloride

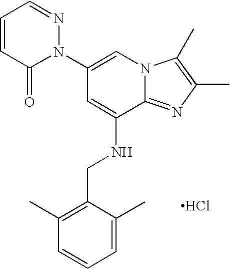

To a solution of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜0.56 mmol; crude product from Description 45) in dichloromethane (5 ml) was added 3(2H)-pyridazinone (150 mg, 1.56 mmol), copper acetate (102 mg, 0.56 mmol), pyridine (46 ul, 0.56 mmol), triethylamine (78 ul, 0.56 mmol) and 4 Angstrom powdered molecular sieves (150 mg). The reaction mixture was stirred at room temperature for 4 hours and then a further quantity of 3(2H)-pyridazinone (100 mg) was added. The mixture was stirred for a further 2 days. The mixture was loaded onto an Isolute® SCX cartridge, eluting with methanol, then 2M NH₃ in methanol. Purification by chromatography on silica gel (ethyl acetate/hexane) gave the free base of the title compound. The product was combined with that of two similar reactions and then dissolved in methanol (5 ml) and treated with 1M HCl in diethyl ether (1 ml). After stirring for 10 minutes, the solvents were evaporated and the resulting solid triturated with diethyl ether to yield the title compound. MS (ES+ve): [M+H]⁺ at m/z 374 (C₂₂H₂₃N₅ O requires [M+H]⁺ at m/z 374).

### EXAMPLE 73

### 1-(8-{[(2,6-Dimethylphenyl)methyl]amino}-2,3-dimethylimidazo[1,2-a]pyridin-6-yl)-4-methyl-1,2-dihydro-3H-pyrazol-3-one hydrochloride

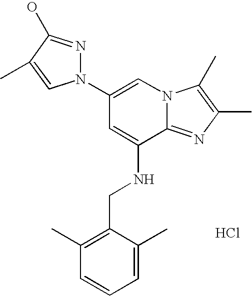

A mixture of 6-bromo-N-[(2,6-dimethylphenyl)methyl]-2,3-dimethylimidazo[1,2-a]pyridin-8-amine (200 mg, 0.56 mmol; WO 98/37080), 4-methyl-2-pyrazolin-5-one (108 mg, 1.1 mmol), copper(I) iodide (9 mg, 0.047 mmol), potassium carbonate (156 mg, 1.1 mol) and N-methylpyrrolidinone (4 mL) was heated in an Initiator™ Microwave Synthesizer at 195° C. for 3 hours. The resulting reaction mixture was applied to an Isolute® SCX cartridge. Elution with methanol, then 2M NH₃ in methanol gave, after evaporation, the crude product which was further purified by triturating with dichloromethane. The product was dissolved in methanol (2 mL) and then 1MHCl in diethyl ether (1 mL) was added. After stirring for 5 minutes, the solvents were evaporated. The product was re-suspended in methanol/diethyl ether and the resulting solid isolated by filtration to give the title compound. MS (ES+ve): [M+H]⁺ at m/z 376 (C₂₂H₂₅ N₅O requires [M+H]⁺ at m/z 376).

### EXAMPLE 74

### 5-(8-{[(2,6-Dimethylphenyl)methyl]amino}-2,3-dimethylimidazo[1,2-a]pyridin-6-yl)-2,4(1H,3H)-pyrimidinedione hydrochloride

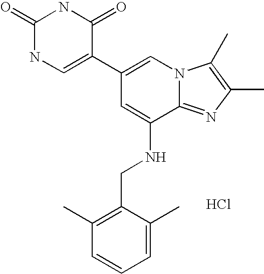

A mixture of 6-bromo-N-[(2,6-dimethylphenyl)methyl]-2,3-dimethylimidazo[1,2-a]pyridin-8-amine (200 mg, 0.56 mmol; WO 98/37080) and 2,4-dioxo-1,2,3,4-tetrahydro-5-pyrimidinyl boronic acid (174 mg, 1.11 mmol) in 2M aqueous sodium carbonate (1 mL) and dimethylformamide (2 mL) was degassed with argon and then treated with, [1,1′-bis(diphenylphosphino)ferrocene]dichloropalladium (5 mg, 0.01 mmol). The reaction was heated in an Initiator™ Microwave Synthesizer at 100° C. for 3 hours. The resulting mixture was applied to an Isolute® SCX cartridge. Elution with methanol, then 2M NH₃ in methanol gave, after evaporation, the crude product which was further purified by silica gel chromatography eluting with methanol/ethyl acetate mixtures. The product was dissolved in methanol (3 mL) and then 1MHCl in diethyl ether (1 mL) was added. After stirring for 5 minutes, the solvents were evaporated to yield the title compound. MS (ES+ve): [M+H]⁺ at m/z 390 (C₂₂H₂₃N₅O₂ requires [M+H]⁺ at m/z 390).

### EXAMPLE 75

### N-[(2,6-Dimethylphenyl)methyl]-2,3-dimethyl-6-[2-(methyloxy)-5-pyrimidinyl]imidazo[1,2-a]pyridin-8-amine

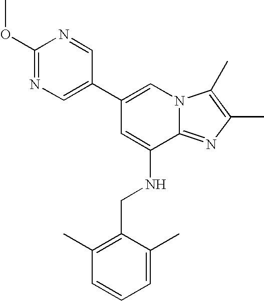

A mixture of 6-bromo-N-[(2,6-dimethylphenyl)methyl]-2,3-dimethylimidazo[1,2-a]pyridin-8-amine (500 mg, 1.39 mmol; WO 98/37080) and 2-methoxypyrimidine-5-boronic acid (430 mg, 2.79 mmol) in 2M aqueous sodium carbonate (3 mL) and dimethylformamide (6 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 2 hours. The resulting reaction mixture was applied to an Isolute® SCX cartridge. Elution with methanol, then 2M and 7M NH₃ in methanol gave, after evaporation, the crude product which was further purified by silica gel chromatography eluting with ethyl acetate/hexane mixtures to yield the title compound. MS (ES+ve): [M+H]⁺ at m/z 388 (C₂₃H₂₅N₅O requires [M+H]⁺ at m/z 388).

### EXAMPLE 76

### 5-(8-{[(2,6-Dimethylphenyl)methyl]amino}-2,3-dimethylimidazo[1,2-a]pyridin-6-yl)-2(1H)-pyrimidinone hydrochloride

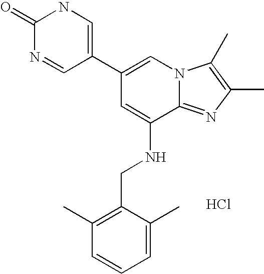

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜0.7 mmol; crude product from Description 45) and 5-bromo-2-hydroxypyrimidine (150 mg, 0.86 mmol) in 2M aqueous sodium carbonate (1.2 mL) and dimethylformamide (2.5 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 2 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/methanol mixtures. The product was further purified by MDAP and then applied to an Isolute® SCX cartridge. Elution with methanol, then 2M NH₃ in methanol gave, after evaporation, the title compound as a free base. The product was dissolved in methanol (5 mL) and then 1M HCl in diethyl ether (1 mL) was added. After stirring for 10 minutes, the solvents were evaporated and then the resulting solid was triturated with diethyl ether to yield the title compound. MS (ES+ve): [M+H]⁺ at m/z 374 (C₂₂H₂₃N₅O requires [M+H]⁺ at m/z 374).

### EXAMPLE 77

### 5-(8-{[(2,6-Dimethylphenyl)methyl]amino}-2,3-dimethylimidazo[1,2-a]pyridin-6-yl)-6-methyl-2,4(1H,3H)-pyrimidinedione hydrochloride

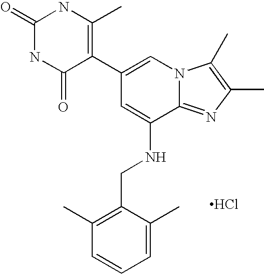

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜0.7 mmol; crude product from Description 45) and 5-bromo-6-methyluracil (170 mg, 0.83 mmol) in 2M aqueous sodium carbonate (1.2 mL) and dimethylformamide (2.5 mL) was degassed with argon and then treated with [1,1 ′-bis(diphenylphosphino)ferrocene]dichloropalladium (10 mg, 0.01 mmol). The mixture was heated in an Initiator™ Microwave Synthesizer at 100° C. for 2 hours. A further aliquot of [1,1′-bis(diphenylphosphino)ferrocene]dichloropalladium (10 mg, 0.01 mmol) was added and the mixture heated in the microwave at 100° C. for a further 2 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/methanol mixtures. The product was dissolved in methanol (3 mL) and then 1M HCl in diethyl ether (0.5 mL) was added. After stirring for 10 minutes, the solvents were evaporated to yield the title compound. MS (ES+ve): [M+H]⁺ at m/z 404 (C₂₃H₂₅N₅O₂ requires [M+H]⁺ at m/z 404).

### EXAMPLE 78

### 5-(8-{[(2,6-Dimethylphenyl)methyl]amino}-2,3-dimethylimidazo[1,2-a]pyridin-6-yl)-1-methyl-2,4(1H,3H)-pyrimidinedione hydrochloride

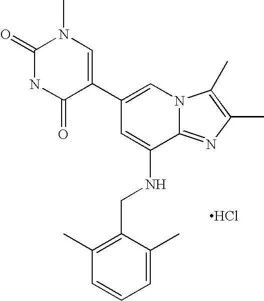

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜1.4 mmol; crude product from Description 45) and 5-bromo-1-methyl-2,4(1H,3H)-pyrimidinedione (251 mg, 1.2 mmol; O. Tee et al., J. Org. Chem., 45, 5, 1980, 830) in 2M aqueous sodium carbonate (1.5 mL) and dimethylformamide (3 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 2 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/hexane mixtures. The product was dissolved in methanol (5 mL) and then 1M HCl in diethyl ether (0.5 mL) was added. After stirring for 10 minutes, the solvents were evaporated to yield the title compound. MS (ES+ve): [M+H]⁺ at m/z 404 (C₂₃H₂₅N₅O₂ requires [M+H]⁺ at m/z 404).

### EXAMPLE 79

### 5-(8-{[(2,6-Dimethylphenyl)methyl]amino}-2,3-dimethylimidazo[1,2-a]pyridin-6-yl)-13-dimethyl-2,4(1H,3H)-pyrimidinedione hydrochloride

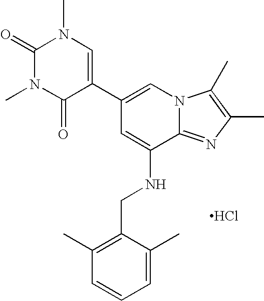

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜1.4 mmol; crude product from Description 45) and 5-bromo-1,3-dimethyluracil (310 mg, 1.4 mmol) in 2M aqueous sodium carbonate (1.5 mL) and dimethylformamide (3 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 2 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/hexane mixtures. The product was dissolved in methanol (5 mL) and then 1M HCl in diethyl ether (0.5 mL) was added. After stirring for 10 minutes, the solvents were evaporated to yield the title compound. MS (ES+ve): [M+H]⁺ at m/z 418 (C₂₄H₂₇N₅O₂ requires [M+H]⁺ at m/z 418).

### EXAMPLE 80

### 2-(8-{[(2,6-Dimethylphenyl)methyl]oxy}-2,3-dimethylimidazo[1,2-a]pyridin-6-yl)-3(2H)-pyridazinone

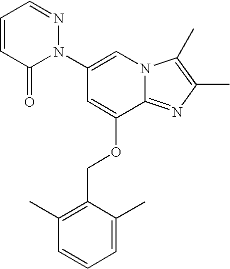

To a solution of 2-(8-hydroxy-2,3-dimethylimidazo[1,2-a]pyridin-6-yl)-3(2H)-pyridazinone (175 mg, 0.68 mmol; Description 48) in dimethylformamide (5 mL) was added potassium carbonate (190 mg, 1.38 mmol) and 2,6-dimethylbenzyl bromide (170 mg, 0.85 mmol). The mixture was stirred at room temperature for 2 hours under argon and then loaded onto an Isolute® SCX cartridge. Elution with methanol, then 2M NH₃ in methanol gave, after evaporation, the crude product which was purified by silica gel chromatography eluting with ethyl acetate to yield the title compound. MS (ES+ve): [M+H]⁺ at m/z 375 (C₂₂H₂₂N₄ O₂ requires [M+H]⁺ at m/z 375).

### EXAMPLE 81

### 6-(8-{[(2,6-Dimethylphenyl)methyl]amino}-2,3-dimethylimidazo[1,2-a]pyridin-6-yl)-2(1H)-pyrazinone hydrochloride

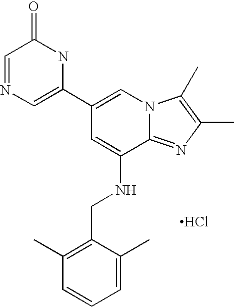

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜1.4 mmol; crude product from Description 45) and 6-chloro-2(1H)-pyrazinone {Cheeseman et al., J. Chem. Soc. C, 1971, 2977} (200 mg, 1.5 mmol) in 2M aqueous sodium carbonate (1.5 mL) and dimethylformamide (3 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 3 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/hexane mixtures. The product was dissolved in methanol (5 mL) and then 1M HCl in diethyl ether (0.5 mL) was added. After stirring for 5 minutes, the solvents were evaporated to yield the title compound. MS (ES+ve): [M+H]⁺ at m/z 374 (C₂₂H₂₃N₅O requires [M+H]⁺ at m/z 374).

### EXAMPLE 82

### N-[(2,6-Dimethylphenyl)methyl]-2,3-dimethyl-6-[4-(methyloxy)-3-pyridinyl]imidazo[1,2-a]pyridin-8-amine

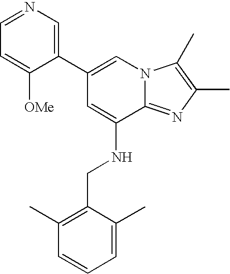

A mixture of 6-bromo-N-[(2,6-dimethylphenyl)methyl]-2,3-dimethylimidazo[1,2-a]pyridin-8-amine (500 mg, 1.4 mmol; WO 98/37080) and 4-methoxy-3-pyridylboronic acid hydrate (420 mg, 2.7 mmol) in 2M aqueous sodium carbonate (1.5 mL) and dimethylformamide (3 mL) was degassed with argon and then treated with, [1,1′-bis(diphenylphosphino)ferrocene]dichloropalladium (10 mg, 0.01 mmol). The reaction was heated in an Initiator™ Microwave Synthesizer at 100° C. for 1 hour. The resulting mixture was applied to an Isolute® SCX cartridge. Elution with methanol, then 2M NH₃ in methanol gave, after evaporation, the crude product which was further purified by silica gel chromatography eluting with ethyl acetate/hexane mixtures to yield the title compound. MS (ES+ve): [M+H]⁺ at m/z 387 (C₂₄H₂₆N₄O requires [M+H]⁺ at m/z 387).

### EXAMPLE 83

### 2-(8-{[(2,6-Dimethylphenyl)methyl]amino}-2,3-dimethylimidazo[1,2-a]pyridin-6-yl)-4(1H)-pyridinone hydrochloride

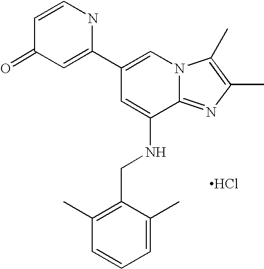

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜1.4 mmol; crude product from Description 45) and 2-bromo-4-hydroxypyridine (250 mg, 1.4 mmol) in 2M aqueous sodium carbonate (1.5 mL) and dimethylformamide (3 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 2 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/methanol mixtures. The product was further purified by MDAP and then applied to an Isolute® SCX cartridge. Elution with methanol, then 2M NH₃ in methanol gave, after evaporation, the title compound as a free base. The product was dissolved in methanol (3 mL) and then 1M HCl in diethyl ether (0.5 mL) was added. After stirring for 5 minutes, the solvents were evaporated to yield the title compound. MS (ES+ve): [M+H]⁺ at m/z 373 (C₂₃H₂₄N₄O requires [M+H]⁺ at m/z 373).

### EXAMPLE 84

### 1-(8-{[(2,6-Dimethylphenyl)methyl]amino}-2,3-dimethylimidazo[1,2-a]pyridin-6-yl)-2-oxo-1,2-dihydro-4-pyridinecarboxamide

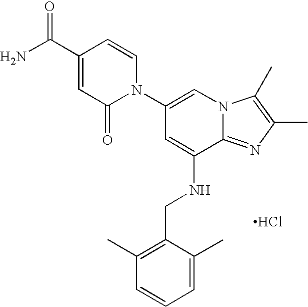

A mixture of 6-bromo-N-[(2,6-dimethylphenyl)methyl]-2,3-dimethylimidazo[1,2-a]pyridin-8-amine (500 mg, 1.4 mmol; WO 98/37080), 2-hydroxyisonicotinonitrile (335 mg, 2.8 mmol), copper(I) iodide (53 mg, 0.27 mmol), potassium carbonate (386 mg, 2.8 mol) and N-methylpyrrolidinone (4 mL) was heated in an Initiator™ Microwave Synthesizer at 150° C. for 16 hours. The resulting reaction mixture was applied to an Isolute® SCX cartridge. Elution with methanol, then 2M NH₃ in methanol gave, after evaporation, the crude product which was further purified by MDAP to yield the free base of the title compound. The product was dissolved in methanol (3 mL) and treated with 1M HCl in diethyl ether (0.5 mL). After stirring for 5 minutes, the solvents were evaporated to give the title compound. MS (ES+ve): [M+H]⁺ at m/z 416 (C₂₄H₂₅ N₅O₂ requires [M+H]⁺ at m/z 416).

### EXAMPLE 85

### 1-(8-{[(2,6-Dimethylphenyl)methyl]amino}-2,3-dimethylimidazo[1,2-a]pyridin-6-yl)-2-oxo-1,2-dihydro-4-pyridinecarbonitrile hydrochloride

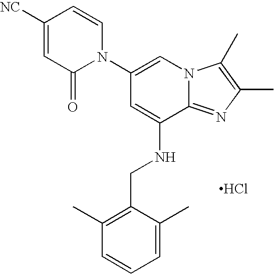

A mixture of 6-bromo-N-[(2,6-dimethylphenyl)methyl]-2,3-dimethylimidazo[1,2-a]pyridin-8-amine (500 mg, 1.4 mmol; WO 98/37080), 2-hydroxyisonicotinonitrile (202 mg, 1.7 mmol), copper(I) iodide (55 mg, 0.28 mmol), potassium phosphate (593 mg, 2.8 mol) and N,N′-dimethylethylenediamine (50 mg, 0.56 mmol) in dioxane(4 mL) was degassed with argon and then heated in an Initiator™ Microwave Synthesizer at 160° C. for 12 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/hexane mixtures. The product was further purified by MDAP and then applied to an Isolute® SCX cartridge. Elution with methanol, then 2M NH₃ in methanol gave, after evaporation, the title compound as a free base. The product was dissolved in methanol (3 mL) and then 1M HCl in diethyl ether (0.5 mL) was added. After stirring for 5 minutes, the solvents were evaporated to yield the title compound. MS (ES+ve): [M+H]⁺ at m/z 398 (C₂₄H₂₃N₅O requires [M+H]⁺ at m/z 398).

### EXAMPLE 86

### 3-(8-{[(2,6-Dimethylphenyl)methyl]amino}-2,3-dimethylimidazo[1,2-a]pyridin-6-yl)-4(1H)-pyridinone hydrochloride

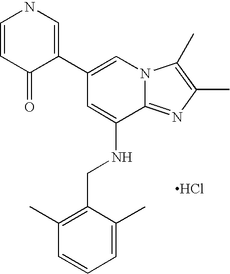

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜1.4 mmol; crude product from Description 45) and 3-bromo-4-hydroxypyridine (250 mg, 1.4 mmol) in 2M aqueous sodium carbonate (1.5 mL) and dimethylformamide (3 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 2 hours. The resulting reaction mixture was applied to an Isolute® SCX cartridge. Elution with methanol, then 2M NH₃ in methanol gave, after evaporation, the crude product which was purified by MDAP. Combined fractions were applied to an Isolute® SCX cartridge. Elution with methanol, then 2M NH₃ in methanol gave, after evaporation, the title compound as a free base. The product was dissolved in methanol (3 mL) and then 1M HCl in diethyl ether (0.5 mL) was added. After stirring for 5 minutes, the solvents were evaporated to yield the title compound. MS (ES+ve): [M+H]⁺ at m/z 373 (C₂₃H₂₄N₄O requires [M+H]⁺ at m/z 373).

### EXAMPLE 87

### 4-(8-{1(2,6-Dimethylphenyl)methyl]amino}-2,3-dimethylimidazo[1,2-a]pyridin-6-yl)-2(1H)-pyridinone hydrochloride

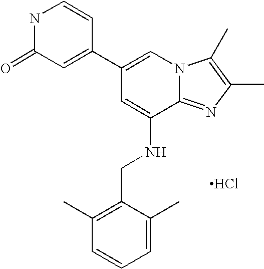

A mixture of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜1.4 mmol; crude product from Description 45) and 4-chloro-2-hydroxypyridine (180 mg, 1.4 mmol) in 2M aqueous sodium carbonate (1.5 mL) and dimethylformamide (3 mL) was degassed with argon and then treated with [1,1′-bis(diphenylphosphino)ferrocene]dichloropalladium (10 mg, 0.01 mmol). The mixture was heated in an Initiator™ Microwave Synthesizer at 100° C. for 2 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/methanol mixtures. The product was further purified by trituration with diethyl ether. The product was dissolved in methanol (3 mL) and then 1M HCl in diethyl ether (0.5 mL) was added. After stirring for 5 minutes, the solvents were evaporated and the resulting product triturated with diethyl ether to yield the title compound. MS (ES+ve): [M+H]⁺ at m/z 373 (C₂₃H₂₄N₄O requires [M+H]⁺ at m/z 373).

### EXAMPLE 88

### 1-(8-{[(2,6-Dimethylphenyl)methyl]amino}-2,3-dimethylimidazo[1,2-a]pyridin-6-yl)-1H-1,2,4-triazole-3-carboxamide hydrochloride

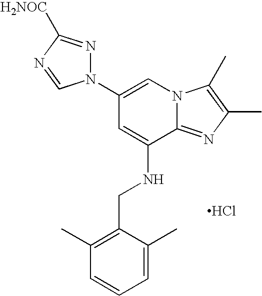

A mixture of 6-bromo-N-[(2,6-dimethylphenyl)methyl]-2,3-dimethylimidazo[1,2-a]pyridin-8-amine (500 mg, 1.4 mmol; WO 98/37080), 3-cyano-1,2,4-triazole (263 mg, 2.8 mmol), copper(I) iodide (27 mg, 0.14 mmol), potassium carbonate (463 mg, 3.4 mol) and N-methylpyrrolidinone (4 mL) was heated in an Initiator™ Microwave Synthesizer at 195° C. for 16 hours. The resulting reaction mixture was applied to an Isolute® SCX cartridge. Elution with methanol, then 2M NH₃ in methanol gave, after evaporation, the crude product which was purified by MDAP. The partially purified product was applied to an Isolute® SCX cartridge. Elution with methanol, then 2M NH₃ in methanol gave, after evaporation, the product which was further purified by silica gel chromatography eluting with ethyl acetate/hexane mixtures. The product was dissolved in methanol (3 mL) and then 1M HCl in diethyl ether (0.5 mL) was added. After stirring for 5 minutes, the solvents were evaporated to give the title compound. MS (ES+ve): [M+H]⁺ at m/z 390 (C₂₁H₂₃ N₇O requires [M+H]⁺ at m/z 390).

### EXAMPLE 89

### 1-(8-{[(2,6-Dimethylphenyl)methyl]amino}-2,3-dimethylimidazo[1,2-a]pyridin-6-yl)-6-oxo-1,6-dihydro-3-pyridinecarbonitrile hydrochloride

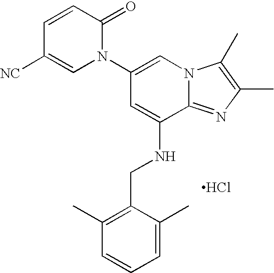

A mixture of 6-bromo-N-[(2,6-dimethylphenyl)methyl]-2,3-dimethylimidazo[1,2-a]pyridin-8-amine (500 mg, 1.4 mmol; WO 98/37080), 6-oxo-1,6-dihydro-3-pyridinecarbonitrile (260 mg, 2.1 mmol; DE 597974), copper(I) iodide (55 mg, 0.28 mmol), potassium phosphate (593 mg, 2.8 mol) and N,N′-dimethylethylenediamine (60 μL, 0.56 mmol) in dioxane (4 mL) was degassed with argon and then heated in an Initiator™ Microwave Synthesizer at 160° C. for 12 hours. The resulting reaction mixture was applied to an Isolute® SCX cartridge. Elution with methanol, then 2M NH₃ in methanol gave, after evaporation, the crude product which was purified by silica gel chromatography eluting with ethyl acetate/hexane mixtures. The product was dissolved in methanol (3 mL) and then 1M HCl in diethyl ether (1 mL) was added. After stirring for 5 minutes, the solvents were evaporated and the resulting solid was triturated with diethyl ether to yield the title compound. MS (ES+ve): [M+H]⁺ at m/z 398 (C₂₄H₂₃N₅O requires [M+H]⁺ at m/z 398).

### EXAMPLE 90

### [1-(8-{[(2,6-Dimethylphenyl)methyl]amino}-2,3-dimethylimidazo[1,2-a]pyridin-6-yl)-1H-pyrazol-4-yl]methanol hydrochloride

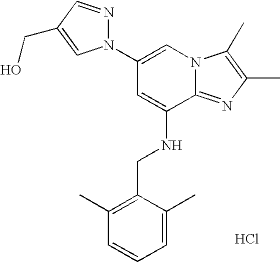

A mixture of ethyl 1-(8-{[(2,6-dimethylphenyl)methyl]amino}-2,3-dimethylimidazo[1,2-a]pyridin-6-yl)-1H-pyrazole-4-carboxylate (57 mg, 0.14 mmol; Description 49), 2M lithium borohydride in tetrahydrofuran (137 μL, 0.27 mmol) and dry tetrahydrofuran (3 ml) were heated to reflux under an argon atmosphere for 21 hours. The mixture was allowed to cool to room temperature then partitioned between dichloromethane and water. The organic phase was washed with water, then brine, and then evaporated. The residue was purified by silica gel chromatography (ethyl acetate/hexane). The residue was dissolved in dichloromethane (1 ml) and treated with 1M HCl in diethyl ether (1 ml). The solvent was evaporated to give the title compound. MS (ES+ve): [M+H]⁺ at m/z 376 (C₂₂H₂₅N₅O requires [M+H]⁺ at m/z 376).

### EXAMPLE 91

### 2-(8-{[(2-Ethyl-6-methylphenyl)methyl]oxy}-2,3-dimethylimidazo[1,2-a]pyridin-6-yl)-3(2H)-pyridazinone hydrochloride

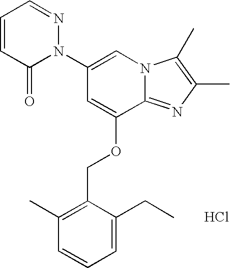

A mixture of 2-(8-hydroxy-2,3-dimethylimidazo[1,2-a]pyridin-6-yl)-3(2H)-pyridazinone (183 mg, 0.71 mmol; Description 48), 2-(bromomethyl)-1-ethyl-3-methylbenzene (183 mg, 0.86 mmol), potassium iodide (47 mg, 0.28 mmol) sodium carbonate (273 mg, 2.6 mmol) and dimethyl formamide (7 ml) was stirred at room temperature for 16 hours. A second portion of 2-(bromomethyl)-1-ethyl-3-methylbenzene (183 mg, 0.86 mmol) was added and the mixture stirred at room temperature for a further 24 hours. The mixture was partitioned between ethyl acetate and water, the organic layer was separated and evaporated. The residue was purified by silica gel chromatography (ethyl acetate). The residue was dissolved in dichloromethane (1 ml) and treated with 1M HCl in diethyl ether (1 ml). The solvent was evaporated to give the title compound. MS (ES+ve): [M+H]⁺ at m/z 389 (C₂₃H₂₄N₄O₂ requires [M+H]⁺ at m/z 389).

### EXAMPLE 92

### 6-(8-{[(2,6-Dimethylphenyl)methyl]amino}-2,3-dimethylimidazo[1,2-a]pyridin-6-yl)-2(1H)-pyridinone hydrochloride

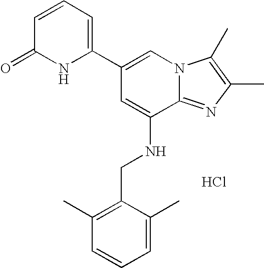

To a solution of N-[(2,6-dimethylphenyl)methyl]-2,3-dimethyl-6-(4,4,5,5-tetramethyl-1,3,2-dioxaborolan-2-yl)imidazo[1,2-a]pyridin-8-amine (˜0.58 mmol; Description 45) in dimethylformamide (1.5 ml) was added [1,1′-bis(diphenylphosphino)ferrocene]dichloropalladium (24 mg, 0.029 mmol), 6-bromo-2(1H)-pyridinone (91 mg, 0.52 mmol), 2M sodium carbonate (0.75 ml, 1.5 mmol) and the reaction mixture heated in an Initiator™ Microwave Synthesizer at 200° C. for 2 minutes. The reaction was allowed to cool, dimethylformamide (1.0 ml) added and the reaction mixture heated in an Initiator™ Microwave Synthesizer at 100° C. for 2 hours. The cooled mixture was partitioned between ethyl acetate and water, washed with water, then brine, dried (MgSO₄) and evaporated. The residue was purified by chromatography on silica gel (ethyl acetate/hexane). The purified solid was dissolved in a mixture of dichloromethane (1 ml) and methanol (1 ml) and 1M HCl in diethyl ether added. The solution was mixed thoroughly and evaporated to give the title compound. MS (ES+ve): [M+H]⁺ at m/z 373 (C₂₃H₂₄N₄O requires [M+H]⁺ at m/z 373).

### EXAMPLE 93

### N-[(2-Ethyl-6-methylphenyl)methyl]-2,3-dimethyl-6-(4-methyl-1,3-oxazol-2-yl)imidazo[1,2-a]pyridin-8-amine hydrochloride

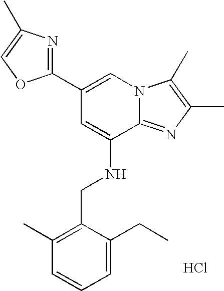

A mixture of 8-{[(2-ethyl-6-methylphenyl)methyl]amino}-2,3-dimethylimidazo[1,2-a]pyridine-6-carboxamide (100 mg, 0.30 mmol; WO 99/55706) and 1-bromo-2,2-dimethoxypropane (1.1 g, 6.0 mmol) was stirred in an oil bath at 130° C. for 2 h. After cooling to room temperature, the reaction mixture was partitioned between ethyl acetate and water. The aqueous phase was extracted with a further portion of ethyl acetate, and the combined organic phases were washed with water, then brine, dried (MgSO₄) and evaporated. The residue was purified by chromatography on silica gel (ethyl acetate/hexane). The product was dissolved in methanol (2 ml), water (0.5 ml) and 2N HCl (0.1 ml) were added. After stirring for 5 minutes, the mixture was evaporated to give the title compound as a buff solid. MS (ES+ve): [M+H]⁺ at m/z 375 (C₂₃H₂₆N₄O requires [M+H]⁺ at m/z 375).

### EXAMPLE 94

### N-[(2,6-Dimethylphenyl)methyl]-2,3-dimethyl-6-[6-(methyloxy)-3-pyridinyl]imidazo[1,2-a]pyridin-8-amine

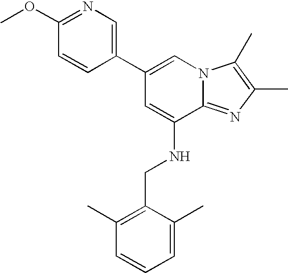

A mixture of 6-bromo-N-[(2,6-dimethylphenyl)methyl]-2,3-dimethylimidazo[1,2-a]pyridin-8-amine (100 mg, 0.28 mmol; WO 98/37080), [6-(methyloxy)-3-pyridinyl]boronic acid (60 mg, 0.39 mmol), [1,1′-bis(diphenylphosphino)ferrocene]dichloropalladium (4 mg, 0.0076 mmol), 2M aqueous sodium carbonate (0.7 ml) and dimethylformamide (1.3 ml) was heated in an Initiator™ Microwave Synthesizer at 100° C. for 30 minutes. The reaction was repeated under the same conditions with 6-bromo-N-[(2,6-dimethylphenyl)methyl]-2,3-dimethylimidazo[1,2-a]pyridin-8-amine (200 mg, 0.56 mmol; WO 98/37080), [6-(methyloxy)-3-pyridinyl]boronic acid (120 mg, 0. 0.785 mmol), [1,1′-bis(diphenylphosphino)ferrocene]dichloropalladium (8 mg, 0.0152 mmol), 2M aqueous sodium carbonate (1 ml) and dimethylformamide (2 ml). The cooled mixtures were combined and partitioned between ethyl acetate and water. The organic phase was washed with water, then brine, dried (MgSO₄) and evaporated. Purification by chromatography on silica gel (ethyl acetate/hexane) gave the title compound as a buff solid. MS (ES+ve): [M+H]⁺ at m/z 387 (C₂₄H₂₆N₄O requires [M+H]⁺ at m/z 387).

### EXAMPLE 95

### 5-(8-{[(2,6-Dimethylphenyl)methyl]amino}-2,3-dimethylimidazo[1,2-a]pyridin-6-yl)-2(1H)-pyridinone hydrochloride

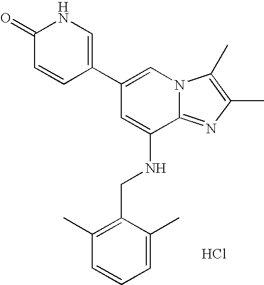

A mixture of N-[(2,6-dimethylphenyl)methyl]-2,3-dimethyl-6-[6-(methyloxy)-3-pyridinyl]imidazo[1,2-a]pyridin-8-amine (106.8 mg, 0.276 mmol; Example 94) and 5N HCl (5 ml) was heated under reflux overnight. The cooled reaction mixture was evaporated to dryness. The residue was dissolved in methanol and water and loaded onto an Isolute® SCX cartridge, elution with methanol, then 1M NH₃ in methanol gave 5-(8-amino-2,3-dimethylimidazo[1,2-a]pyridin-6-yl)-2(1H)-pyridinone; MS (ES+ve): [M+H]⁺ at m/z 255 (C₁₄H₁₄N₄O requires [M+H]⁺ at m/z 255). This intermediate (60 mg, 0.23 mmol) was dissolved in dimethylformamide (3 ml), 2,6-dimethylbenzyl bromide (45 mg, 0.23 mmol) and sodium carbonate (48 mg, 0.45 mmol) were added and the mixture stirred at room temperature overnight. The reaction mixture was partitioned between ethyl acetate and water. And further extracted with ethyl acetate. The organic phase was washed with water, then brine, dried (MgSO₄) and evaporated. The residue was purified by chromatography on silica gel (ethyl acetate/methanol). The product was dissolved in methanol/water/2N HCl (15:35:2 ml) and then loaded onto a 10 g tC18 Sep-Pak® cartridge and eluted with a gradient up to methanol/water/2N HCl (65:35:0.5). Evaporation gave the title compound as a white solid. MS (ES+ve): [M+H]⁺ at m/z 373 (C₂₃H₂₄N₄O requires [M+H]⁺ at m/z 373).

### EXAMPLES 96 AND 97

### N-[(2-Ethyl-6-methylphenyl)methyl]-2,3-dimethyl-6-(1-methyl-1H-1,2,4-triazol-5-yl)imidazo[1,2-a]pyridin-8-amine hydrochloride and N-[(2-ethyl-6-methylphenyl)methyl]-2,3-dimethyl-6-(1-methyl-1H-1,2,4-triazol-3-yl)imidazo[1,2-a]pyridin-8-amine hydrochloride

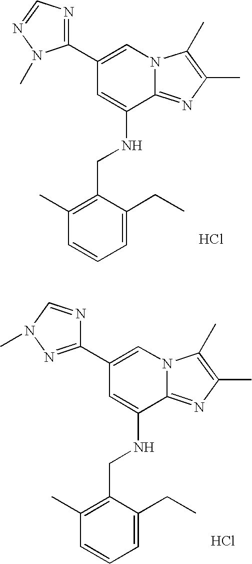

A mixture of N-[(dimethylamino)methylidene]-8-{[(2-ethyl-6-methylphenyl)methyl]amino}-2,3-dimethylimidazo[1,2-a]pyridine-6-carboxamide (185 mg, 0.473 mmol; Description 10), methylhydrazine (0.0506 mL, 0.951 mmol) and acetic acid (6 ml) was heated at 45° C. for 1 hour. After cooling, the mixture was evaporated to dryness. The residue was dissolved in methanol and loaded onto an Isolute® SCX cartridge, elution with methanol, then 1M NH₃ in methanol gave the product as a mixture of isomers. The isomers were separated by chromatography on silica gel (ethyl acetate/methanol). The earlier eluting isomer was dissolved in methanol/water/2N HCl (15:35:2 ml) and then loaded onto a 10 g tC18 Sep-Pak® cartridge and eluted with a gradient up to methanol/water/2N HCl (60:40:0.5). Evaporation gave N-[(2-ethyl-6-methylphenyl)methyl]-2,3-dimethyl-6-(1-methyl-1H-1,2,4-triazol-5-yl)imidazo[1,2-a]pyridin-8-amine hydrochloride as a white solid. MS (ES+ve): [M+H]⁺ at m/z 375 (C₂₂H₂₆N₆ requires [M+H]⁺ at m/z 375). The later eluting isomer was dissolved in methanol/water/2N HCl (15:35:2 ml) and then loaded onto a 10 g tC18 Sep-Pak® cartridge and eluted with a gradient up to methanol/water/2N HCl (65:35:0.5). Evaporation gave N-[(2-ethyl-6-methylphenyl)methyl]-2,3-dimethyl-6-(1-methyl-1H-1,2,4-triazol-3-yl)imidazo[1,2-a]pyridin-8-amine hydrochloride as a white solid. MS (ES+ve): [M+H]⁺ at m/z 375 (C₂₂H₂₆N₆ requires [M+H]⁺ at m/z 375).

### EXAMPLE 98

### 1-(8-{[(2,6-Dimethylphenyl)methyl]amino}-2,3-dimethylimidazo[1,2-a]pyridin-6-yl)-6-oxo-1,6-dihydro-3-pyridinecarboxamide hydrochloride

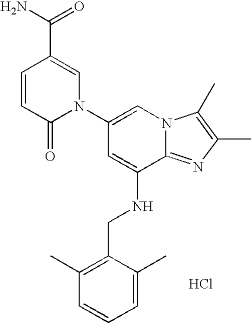

To a mixture of 1-(8-{[(2,6-dimethylphenyl)methyl]amino}-2,3-dimethylimidazo[1,2-a]pyridin-6-yl)-6-oxo-1,6-dihydro-3-pyridinecarboxylic acid hydrochloride (53 mg, 0.117 mmol; Description 51), 1-hydroxybenzotriazole ammonium salt (27 mg, 0.176 mmol; Description 14) and N-ethylmorpholine (0.061 ml, 0.479 mmol) in dimethylformamide (2 mL), was added 1-(3-dimethylaminopropyl)-3-ethylcarbodiimide hydrochloride (28 mg, 0.146 mmol). The mixture was stirred at room temperature for 2 hours, and then diluted with water. The mixture was acidified with 2N HCl and then loaded onto a 10 g tC18 Sep-Pak® cartridge and eluted with a gradient up to methanol/water/2N HCl (60:40:0.5) to give the title compound as a white solid. MS (ES+ve): [M+H]⁺ at m/z 416 (C₂₄H₂₅N₅O₂ requires [M+H]⁺ at m/z 416).

### EXAMPLE 99

### 1-(8-{[(2,6-dimethylphenyl)methyl]amino}-2,3-dimethylimidazo[1,2-a]pyridin-6-yl)-2-oxo-1,2-dihydro-3-pyridinecarbonitrile hydrochloride

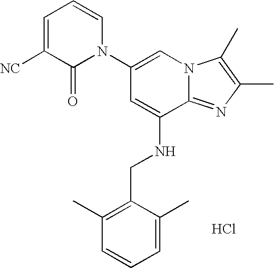

A mixture of 6-bromo-N-[(2,6-dimethylphenyl)methyl]-2,3-dimethylimidazo[1,2-a]pyridin-8-amine (300 mg, 0.837 mmol; WO 98/37080), 3-cyano-2-hydroxypyridine (120 mg, 1.0 mmol), copper(I) iodide (33 mg, 0.173 mmol), potassium phosphate (354 mg, 1.67 mol), N,N′-dimethylethylenediamine (30 mg, 0.34 mmol) and dioxane (4 ml) was heated in an Initiator™ Microwave Synthesizer at 160° C. for 13 hours. The cooled reaction mixture was partitioned between ethyl acetate and water. The solid that was present was collected by filtration. The organic phase was washed with water, then brine, dried (MgSO₄) and evaporated. The residue was purified by chromatography on silica gel (ethyl acetate/toluene) and then combined with the previously isolated solid. This product was dissolved in methanol/water/2N HCl (20:40:2 ml) and then loaded onto a 10 g tC18 Sep-Pak® cartridge and eluted with a gradient up to methanol/water/2N HCl (75:25:0.5) to give the title compound as a solid. MS (ES+ve): [M+H]⁺ at m/z 398 (C₂₄H₂₃N₅O requires [M+H]⁺ at m/z 398).

### EXAMPLE 100

### 1-(8-{[(2,6-Dimethylphenyl)methyl]amino}-2,3-dimethylimidazo[1,2-a]pyridin-6-yl)-3-methyl-2(1H)-pyridinone hydrochloride

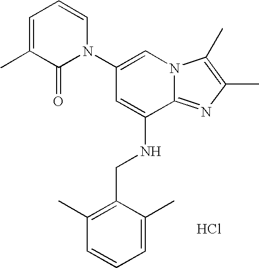

A mixture of 6-bromo-N-[(2,6-dimethylphenyl)methyl]-2,3-dimethylimidazo[1,2-a]pyridin-8-amine (100 mg, 0.28 mmol; WO 98/37080), 2-hydroxy-3-methylpyridine (61 mg, 0.56 mmol), copper(I) iodide (10.6 mg, 0.056 mmol), potassium carbonate (77 mg, 0.56 mol) and dimethylformamide (2 mL) was heated in an Initiator™ Microwave Synthesizer at 150° C. for 16 hours. The cooled mixture was partitioned between ethyl acetate (3×10 ml) and water (15 ml). The combined organic extracts were washed with brine (3×20 ml) and dried (Na₂SO₄). The solvent was evaporated and the residue purified by chromatography on silica. Elution with dichloromethane/methanol (0 to 10%) gave a pale yellow solid which was dissolved in dichloromethane (1 mL). 1M HCl in diethylether (0.5 ml) was added and the solvent evaporated. The residue was triturated under diethylether (1 mL) and filtered to give the title compound as a buff solid. MS (ES+ve): [M+H]⁺ at m/z 387 (C₂₄H₂₆N₄O requires [M+H]⁺ at m/z 387).

### EXAMPLE 101

### 1-(8-{[(2,6-Dimethylphenyl)methyl]amino}-2,3-dimethylimidazo[1,2-a]pyridin-6-yl)-5-methyl-2(1H)-pyridinone hydrochloride

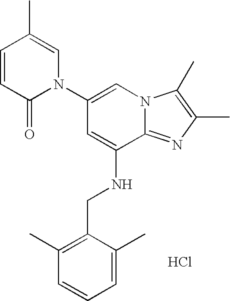

A mixture of 6-bromo-N-[(2,6-dimethylphenyl)methyl]-2,3-dimethylimidazo[1,2-a]pyridin-8-amine (150 mg, 0.419 mmol; WO 98/37080), 2-hydroxy-5-methylpyridine (100 mg, 0.838 mmol), copper(I) iodide (20 mg, 0.1 mmol), potassium carbonate (115 mg, 0.83 mol) and dimethylformamide (3 mL) was heated in an Initiator™ Microwave Synthesizer at 150° C. for 16 hours. The cooled mixture was applied to an Isolute® SCX cartridge. Elution with methanol, then 1M NH₃ in methanol gave, after evaporation, the product which was further purified by chromatography on silica gel. Elution with dichloromethane/methanol (0 to 10%) gave a pale yellow solid which was dissolved in methanol (2 mL), 1M HCl in diethylether (0.5 ml) was added and the solvent evaporated. The residue was triturated under diethylether (1 mL) and filtered to give the title compound as a pale yellow solid. MS (ES+ve): [M+H]⁺ at m/z 387 (C₂₄H₂₆N₄O requires [M+H]⁺ at m/z 387).

### EXAMPLES 102 AND 103

### [1-(8-{[(2,6-dimethylphenyl)methyl]amino}-2,3-dimethylimidazo[1,2-a]pyridin-6-yl)-1H-1,2,4-triazol-3-yl]methanol (A) and [1-(8-{[(2,6-dimethylphenyl)methyl]amino}-2,3-dimethylimidazo[1,2-a]pyridin-6-yl)-1H-1,2,4-triazol-5-yl]methanol (B)

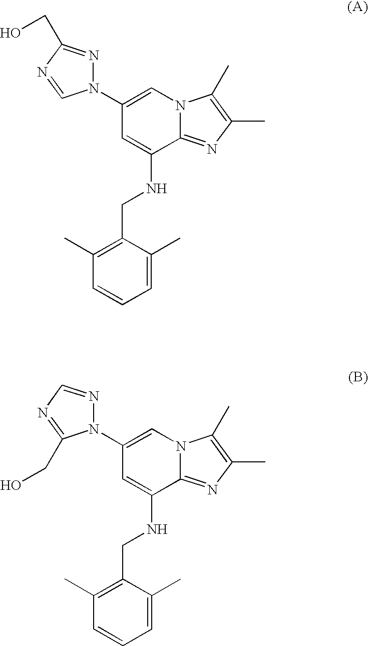

A mixture of 6-bromo-N-[(2,6-dimethylphenyl)methyl]-2,3-dimethylimidazo[1,2-a]pyridin-8-amine (200 mg, 0.559 mmol; WO 98/37080), 1H-1,2,4-triazol-3-ylmethanol (110 mg, 1.12 mmol), copper(I) iodide (32 mg, 0.167 mmol), N,N′-dimethylethylenediamine (15 mg, 0.167 mmol) and cesium carbonate (382 mg, 1.12 mmol) and N-methyl-2-pyrrolidone (2 mL) was heated in an Initiator™ Microwave Synthesizer at 150° C. for 16 hours. The cooled mixture was applied to an Isolute® SCX cartridge, eluting with methanol, then 1M NH₃ in methanol followed by further purification by chromatography on silica gel. Elution with dichloromethane/methanol (5%) gave the title compound (A) as a pale brown solid. MS (ES+ve): [M+H]⁺ at m/z 377 (C₂₁H₂₄N₆O requires [M+H]⁺ at m/z 377) and the title compound (B) as a pale brown solid MS (ES+ve): [M+H]⁺ at m/z 377 (C₂₁H₂₄N₆O requires [M+H]⁺ at m/z 377)

### EXAMPLE 104

### 1-(8-{[(2-Ethyl-6-methylphenyl)methyl]oxy}-2,3-dimethylimidazo[1,2-a]pyridin-6-yl)-2(1)-pyridinone

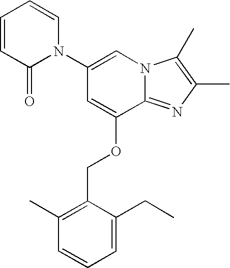

A mixture of 1-(8-hydroxy-2,3-dimethylimidazo[1,2-a]pyridin-6-yl)-2(1H)-pyridinone (50 mg, 0.2 mmol; Description 23), 2-ethyl-6-methylbenzyl bromide (62 mg, 0.29 mmol; Description 6), and potassium carbonate (40 mg, 0.28 mmol) in dimethylformamide (2 mL) was stirred at room temperature for 2 hours. The mixture was applied to an Isolute® SCX cartridge. Elution with methanol, then 1M NH₃ in methanol gave, after evaporation, the product which was further purified by chromatography on silica gel. Elution with dichloromethane/2M ammonia in methanol followed by evaporation gave the title compound. MS (ES+ve): [M+H]⁺ at m/z 388 (C₂₄H₂₅N₃O₂ requires [M+H]⁺ at m/z 388).

### EXAMPLE 105

### 1-(8-{[(2,6-dimethylphenyl)methyl]oxy}-3-ethenyl-2-methylimidazo[1,2-a]pyridin-6-yl)-2(1H)-pyridinone

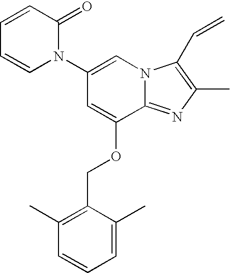

The title compound was obtained from Description 41 in an analogous manner to the process described for the synthesis of Description 38 from Description 37. MS (ES+ve): [M+H-2,6-di-Me-benzyl]⁺ at m/z 268 (C₂₄H₂₃N₃O₂ requires [M+H-2,6-di-Me-benzyl]⁺ at m/z 268).

### EXAMPLE 106

### N-[(1R)-2,3-dihydro-1H-inden-1-yl]-2,3-dimethyl-6-(1H-1,2,4-triazol-1-yl)imidazo[1,2-a]pyridin-8-amine

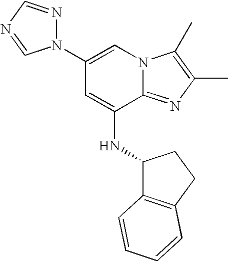

A mixture of 8-bromo-2,3-dimethyl-6-(1H-1,2,4-triazol-1-yl)imidazo[1,2-a]pyridine (Description 52; 133 mg, 0.455 mmol), (1R)-2,3-dihydro-1H-inden-1-amine (178 mg, 1.34 mmol), 2′-dicyclohexylphosphino-2-(N,N-dimethylamino)biphenyl (27 mg, 0.069 mmol), tris(dibenzylideneacetone)dipalladium (0) (21 mg, 0.023 mmol) and sodium t-butoxide (86 mg, 0.90 mmol) in dry dioxane (5 mL) was heated in an Initiator™ Microwave Synthesizer at 120° C. for 40 mins. Further portions of (1R)-2,3-dihydro-1H-inden-1-amine (178 mg, 1.34 mmol), 2′-dicyclohexylphosphino-2-(N,N-dimethylamino)biphenyl (27 mg, 0.069 mmol), tris(dibenzylideneacetone)dipalladium (0) (21 mg, 0.023 mmol) and sodium t-butoxide (86 mg, 0.90 mmol) were added and heating in the microwave continued for a further 40 mins. The solvent was evaporated and the residue loaded onto an Isolute® SCX cartridge, eluting with methanol, then 2M NH₃ in methanol. Fractions containing the product were purified by silica gel chromatography. Elution with dichloromethane/methanol (0% and increasing the polarity to 6%) gave the title compound as a brown oil. MS (ES+ve): [M+H]⁺ at m/z 345 (C₂₀H₂₀N₆ requires [M+H]⁺ at m/z 345).

The following compounds were prepared by reaction of 8-bromo-2,3-dimethyl-6-(1H-1,2,4-triazol-1-yl)imidazo[1,2-a]pyridine (Description 52) with the appropriate amine in an analogous manner to that described for Example 106 altering reaction temperatures, times, solvents and drying agents as appropriate. Examples 107 and 108 were prepared as the free base. Example 109 was converted to the HCl salt using an appropriate additional step.

| | | | Characterisation |
|---|---|---|---|
| | | | MS(ES + ve) |
| | Structure/Name of | | m/z[M + H]⁺ |
| Example No. | product | Amine | observed. |
| Example107. | 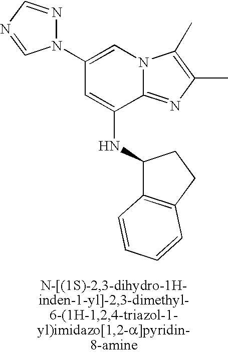 | (1S)-2,3-dihydro-1H-inden-1-amine | 345 |
| Example108. | 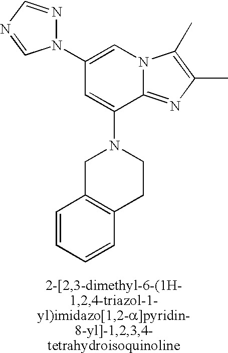 | 1,2,3,4-tetrahydro-isoquinoline | 345 |
| Example109. | 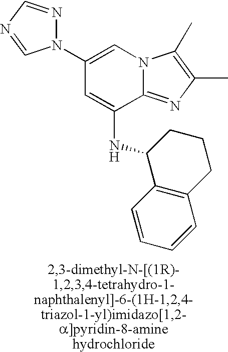 | (R)-1-aminotetralin | 359 |

### EXAMPLE 110

### 2,3-dimethyl-N-[(2-methylphenyl)methyl]-6-(1H-1,2,4-triazol-1-yl)imidazo[1,2-a]pyridin-8-amine hydrochloride

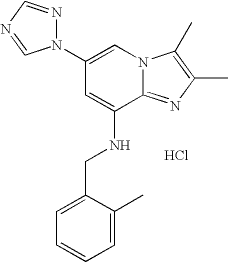

A mixture of 6-bromo-N-[(2-methylphenyl)methyl]-2,3-dimethylimidazo[1,2-a]pyridin-8-amine (Description 53; 261 mg, 0.76 mmol), copper (I) iodide (14 mg, 0.076 mmol), potassium carbonate (210 mg, 1.52 mmol), 1,2,4-triazole (105 mg, 1.52 mmol) and N-methylpyrrolidinone (4.5 ml) was heated in an Initiator™ Microwave Synthesizer at 195° C. for 16 hours. The reaction mixture was partitioned between ethyl acetate and water, the organic phase washed thoroughly with water, saturated lithium chloride solution and brine, dried (MgSO₄) and evaporated. The residue was purified by chromatography on silica gel (ethyl acetate/hexane) and evaporated, redissolved in dichloromethane and treated with 1M HCl in diethyl ether (0.5 ml) and evaporated to give the title compound. MS (ES+ve): [M+H]⁺ at m/z 333 (C₁₉H₂₀N₆ requires [M+H]⁺ at m/z 333).

### EXAMPLE 111

### N-[(2-chloro-4-fluorophenyl)methyl]-2,3-dimethyl-6-(1H-1,2,4-triazol-1-yl)imidazo[1,2-a]pyridin-8-amine

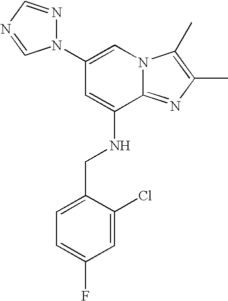

A mixture of 2,3-dimethyl-6-(1H-1,2,4-triazol-1-yl)imidazo[1,2-a]pyridin-8-amine (Description 54; 150 mg, 0.66 mmol), 1-(bromomethyl)-2-chloro-4-fluorobenzene (176 mg, 0.79 mmol) and sodium carbonate (220 mg, 2.1 mmol) were stirred in dimethylformamide (5 ml) for 48 hours, further 1-(bromomethyl)-2-chloro-4-fluorobenzene (147 mg, 0.66 mmol) was added and the reaction stirred for 18 hours and partitioned between ethyl acetate and water. The organic phase was washed with water, brine, dried (MgSO₄), evaporated, purified by chromatography on silica gel (ethyl acetate/hexane) and evaporated to give the title compound. MS (ES+ve): [M+H]⁺ at m/z 371 and 373 (C₁₈H₁₆ClFN₆ requires [M+H]⁺ at m/z 371 and 373).

The following compounds were prepared by reaction of 2,3-dimethyl-6-(1H-1,2,4-triazol-1-yl)imidazo[1,2-a]pyridin-8-amine (Description 54) with the appropriate benzyl bromide (as indicated) in an analogous manner to that described for Example 111 altering reaction temperatures, times, solvents and drying agents as appropriate:

| | | | Characterisation |
|---|---|---|---|
| | | | MS(ES + ve) |
| Example | | | m/z[M + H]⁺ |
| No. | Structure/Name of product | Benzyl bromide | observed. |
| 112 | 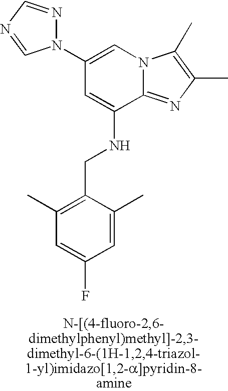 | 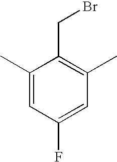 | 365 |
| 113 | 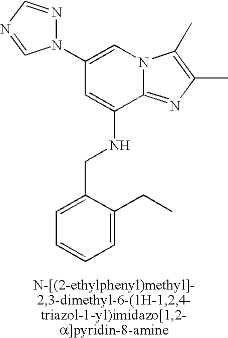 | 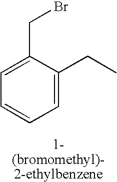 | 347 |
| 114 | 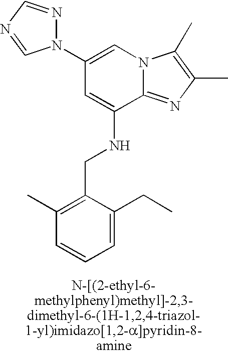 | 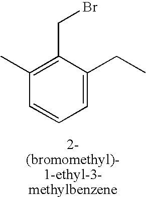 | 361 |
| 115 | 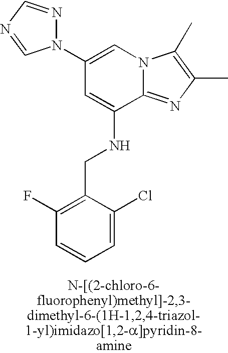 | 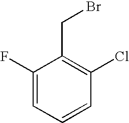 | 371, 373 |
| 116 | 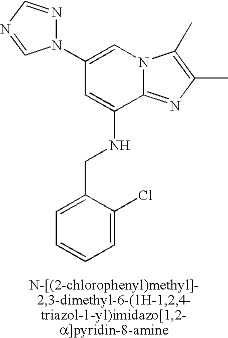 | 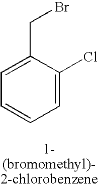 | 353 and 355 |

The following compounds were prepared from 1-(8-amino-2,3-dimethylimidazo[1,2-a]pyridin-6-yl)-2(1H)-pyridinone (Description 19) by reaction with the appropriate alkyl halide for 16 h in a manner analogous to that of example 52 altering reaction temperatures, times, solvents and drying agents as appropriate and omitting the salt-forming step:

| | | | Charater- |
|---|---|---|---|
| | | | isation |
| | | | MS(ES + ve) |
| | | | m/z[M + H]⁺ |
| | Structure/Compound Name | Alkyl halide | observed |
| 117 | 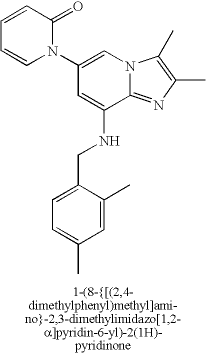 | 1-(bromomethyl)-2,4-dimethyl-benzene | 373 |
| 118 | 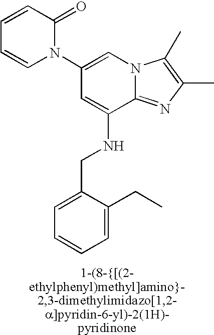 | 1-(bromomethyl)-2-ethylbenzene(3 eq) | 373 |
| 119 | 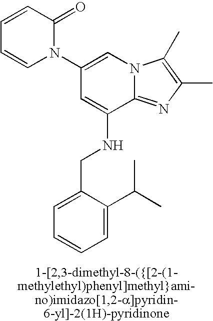 | 1-(bromomethyl)-2-(1-methyl-ethyl)benzene(Reactiontemp. 60° C.) | 387 |

### EXAMPLE 118

### (1-(8-{[(2-ethylphenyl)methyl]amino}-2,3-dimethylimidazo[1,2-a]pyridin-6-yl)-2(1H)-pyridinone) Can Alternatively be Synthesized According to the Following Method:

A mixture of 6-bromo-N-[(2-ethylphenyl)methyl]-2,3-dimethylimidazo[1,2-a]pyridin-8-amine (Description 56, 50 mg, 0.14 mmol), potassium carbonate (38 mg, 0.28 mmol), 2(1H)-pyridinone (27 mg, 0.28 mmol), copper iodide (5 mg, 0.028 mmol) in anhydrous N-methylpyrrolidinone (3 mL) was heated in an Initiator™ Microwave Synthesizer at 195° C. for 16 hours. The mixture was partitioned between ethyl acetate and water, the organic layer evaporated and purified by MDAP. Fractions containing the product were combined and evaporated. The product was re-dissolved into ethyl acetate, washed with sodium bicarbonate and water and all solvents evaporated to give the free base of the title compound. MS (ES+ve): [M+H]⁺ at m/z 373 (C₂₃H₂₄N₄O requires [M+H]⁺ at m/z 373).

### EXAMPLE 120

### 1-{8-[(1S)-2,3-dihydro-1H-inden-1-ylamino]-2,3-dimethylimidazo[1,2-a]pyridin-6-yl}-2(1H)-pyridinone hydrochloride

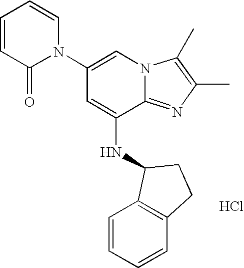

A mixture of 1-(8-bromo-2,3-dimethylimidazo[1,2-a]pyridin-6-yl)-2(1H)-pyridinone (Description 55; 30 mg, 0.09 mmol), (S)-1-aminoindan (31 mg, 0.24 mmol), sodium tert-butoxide (13 mg, 0.13 mmol), 2,2′-bis(diphenylphosphino)-1,1′-binaphthyl (BINAP) (6 mg, 0.01 mmol), tris(dibenzylideneacetone)dipalladium(0) (9 mg, 0.01 mmol), and toluene (3 ml) was heated to 100° C. for 16 hours. The cooled reaction mixture was loaded onto an Isolute® SCX cartridge, washed with methanol and then eluted with 1M NH₃/MeOH. The solvent was evaporated and the residue purified by silica gel chromatography (ethyl acetate). The solid was dissolved in dichloromethane (1 ml) and treated with 1M HCl in diethyl ether (1 ml). The solvent was evaporated to give the title compound. MS (ES+ve): [M+H]⁺ at m/z 371 (C₂₃H₂₂N₄O requires [M+H]⁺ at m/z 371).

### EXAMPLE 121

### (R,S)-1-[8-(2,3-dihydro-1H-inden-1-ylamino)-2,3-dimethylimidazo[1,2-a]pyridin-6-yl]-2(1H)-pyridinone hydrochloride

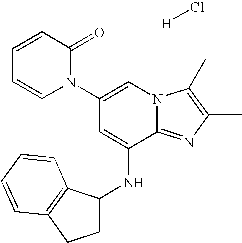

1-(8-amino-2,3-dimethylimidazo[1,2-a]pyridin-6-yl)-2(1H)-pyridinone (Description 19; 292 mg, 1.15 mmol, description 19), sodium carbonate (390 mg, 3.7 mmol), 1-bromo-2,3-dihydro-1H-indene (340 mg, 1.7 mmol; Journal of the American Chemical Society (2005), 127(30), 10482-10483) were combined in anhydrous dimethylformamide (20 mL) and stirred at 60° C. for 16 hours. The mixture was partitioned between ethyl acetate and water, the organic layer evaporated and the residue purified by MDAP. Fractions containing the product were combined and evaporated. The product was re-dissolved into ethyl acetate, washed with sodium bicarbonate and water and the solvent removed. Further purification was undertaken on silica eluting with ethyl acetate as eluant. The solvent was evaporated, additional purification by MDAP and evaporation, with the product was re-dissolved into ethyl acetate, washed with sodium bicarbonate and water and the solvent removed. The residue was dissolved in dichloromethane and 0.5 mL 1M HCl in diethylether was added. The solvent was evaporated to give the title compound.

MS (ES+ve): [M+H]⁺ at m/z 371 (C₂₃H₂₂N₄O requires [M+H]⁺ at m/z 371).

### EXAMPLE 122

### 1-{8-[(1S)-2,3-dihydro-1H-inden-1-ylamino]-2,3-dimethylimidazo[1,2-a]pyridin-6-yl}-2(1H)-pyridinone hydrochloride and

### EXAMPLE 123

### 1-{8-[(1R)-2,3-dihydro-1H-inden-1-ylamino]-2,3-dimethylimidazo[1,2-a]pyridin-6-yl}-2(1H)-pyridinone hydrochloride

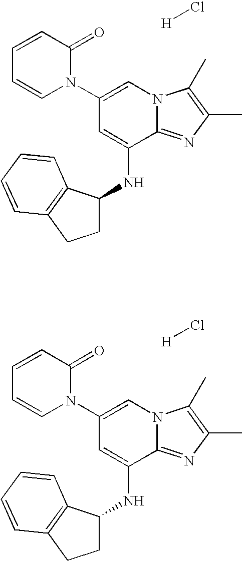

To 1-(8-bromo-2,3-dimethylimidazo[1,2-a]pyridin-6-yl)-2(1H)-pyridinone (Description 55; 431 mg, 1.35 mmol), 2,3-dihydro-1H-inden-1-amine (538 mg, 4.05 mmol), sodium tert-butoxide (181 mg, 1.90 mmol), BINAP (100 mg), Pd2(dba)3 (100 mg) and toluene (30 ml) were stirred at reflux under argon for 16 hours. The mixture was loaded onto an Isolute® SCX cartridge, washing with methanol and then eluted with 2M NH₃/MeOH. Solvent was removed in vacuo from appropriate fractions and the resulting mixture was purified on silica eluting with ethyl acetate. Evaporation gave the racemate which was resolved by chiral HPLC (H_Chiral 1100 UV prep 1 instrument on a Chiralcel OD 20 mm×250 mm 10 uM column eluting with 9:1 heptane:ethanol at 1 mL/min and collecting at 215 nM wavelength) to give the (S) enantiomer as the faster peak and the (R) enantiomer as the slower-running peak. Each enantiomer was then separately dissolved in dichloromethane and 1M HCl in diethylether added and the solvent removed to give the two title compounds.

MS (ES+ve): [M+H]⁺ at m/z 371 (C₂₃H₂₂N₄O requires [M+H]⁺ at m/z 371) for example 122;

MS (ES+ve): [M+H]⁺ at m/z 371 (C₂₃H₂₂N₄O requires [M+H]⁺ at m/z 371) for example 123.

### EXAMPLE 124

### 1-(8-{[(1S,2S)-2-hydroxy-2,3-dihydro-1H-inden-1-yl]amino}-2,3-dimethylimidazo[1,2-a]pyridin-6-yl)-2(1H)-pyridinone and

### EXAMPLE 125

### 1-(8-{[(1R,2R)-2-hydroxy-2,3-dihydro-1H-inden-1-yl]amino}-2,3-dimethylimidazo[1,2-a]pyridin-6-yl)-2(1H)-pyridinone

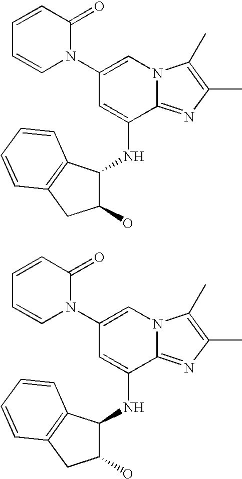

1-(8-Amino-2,3-dimethylimidazo[1,2-a]pyridin-6-yl)-2(1H)-pyridinone (200 mg, 0.79 mmol, description 19), triethylamine (160 mg, 1.58 mmol), 6,6a-dihydro-1aH-indeno[1,2-b]oxirene (956 mg, 7.24 mmol) were combined in 0.4 mL water and 8 mL methanol and stirred at 50° C. for 48 hours. The mixture was loaded onto an Isolute® SCX cartridge, washing with methanol and then eluted with 2M NH₃/MeOH. Solvent was removed in vacuo from appropriate fractions and the resulting mixture was purified on silica eluting with a 0-10% methanol in ethyl acetate gradient. Evaporation yielded the title compound as a mixture of the trans diastereomers.

MS (ES+ve): [M+H]⁺ at m/z 387 (C₂₃H₂₂N₄O₂ requires [M+H]⁺ at m/z 387) for example 124;

MS (ES+ve): [M+H]⁺ at m/z 387 (C₂₃H₂₂N₄O₂ requires [M+H]⁺ at m/z 387) for example 125.

The mixture was separated by HPLC on a H_Chiral 1100 UV prep 1 instrument on a chiralpak AD 20 mm ID×250 mm, 10 uM eluting with 1:1 heptane:ethanol at 17.0 mL/min with 215 nM wavelength detection to give the two trans diastereomers.

MS (ES+ve): [M+H]⁺ at m/z 387 (C₂₃H₂₂N₄O₂ requires [M+H]⁺ at m/z 387).

### EXAMPLE 126

### 1-[8-(3,4-dihydro-2(1H)-isoquinolinyl)-2,3-dimethylimidazo[1,2-a]pyridin-6-yl]-2(1H)-pyridinone hydrochloride

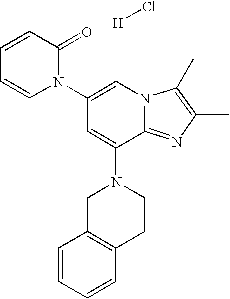

A mixture of 1-(8-bromo-2,3-dimethylimidazo[1,2-a]pyridin-6-yl)-2(1H)-pyridinone (Description 55; 170 mg, 0.38 mmol), sodium tert-butoxide (71 mg, 0.74 mmol), 1,2,3,4-tetrahydroisoquinoline (213 mg, 1.60 mmol), (±) BINAP (20 mg) and Pd₂(dba)₃ (20 mg) in anhydrous toluene (20 mL) were heated at reflux under argon for 16 hours. The mixture was applied to an Isolute® SCX cartridge and eluted with methanol followed by 2M NH₃ in methanol. The basic fractions were combined and evaporated under reduced pressure. This residue was purified by column chromatography on silica eluting ethyl acetate. This product was dissolved in dichloromethane (2 mL), and then 1M HCl in diethyl ether was added. The solvent was evaporated to give the title compound.

MS (ES+ve): [M+H]⁺ at m/z 371 (C₂₃H₂₂N₄O requires [M+H]⁺ at m/z 371).

The following compounds were prepared from 1-(8-bromo-2,3-dimethylimidazo[1,2-a]pyridin-6-yl)-2(1H)-pyridinone (Description 55) by reaction with the appropriate amine in a manner analogous to that of example 126 altering reaction temperatures, times, solvents and drying agents as appropriate. Examples 127 to 129 were prepared as the free base by omitting the salt-forming step of the method of example 126.

| | | | Character- |
|---|---|---|---|
| | | | isation |
| | | | MS(ES + ve) |
| | | | m/z[M + H]⁺ |
| | Structure/Compound Name | Amine | observed |
| 127 | 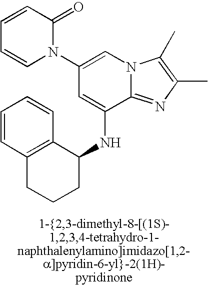 | (1S)-1,2,3,4-tetrahydro-1-naphthalen-amine | 385 |
| 128 | 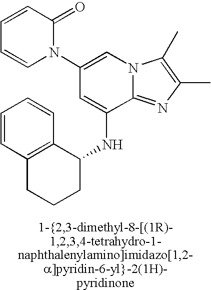 | (1R)-1,2,3,4-tetrahydro-1-naphthalen-amine | 385 |
| 129 | 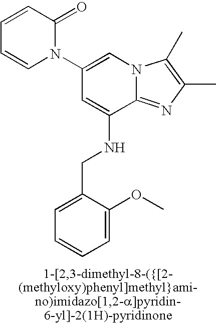 | 1-[2-(methyl-oxy)phenyl]methanamine | 375 |

### EXAMPLE 130

### 1-{8-{[(2,6-dimethylphenyl)methyl]oxy}-2-methyl-3-[(methyloxy)methyl]imidazo[1,2-a]pyridin-6-yl}-2(1)-pyridinone

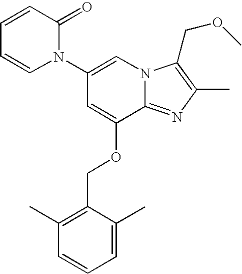

To a solution of 1-[8-{[(2,6-dimethylphenyl)methyl]oxy}-3-(hydroxymethyl)-2-methylimidazo[1,2-a]pyridin-6-yl]-2(1H)-pyridinone (example 70, 77 mg, 0.2 mmol) in dimethylformamide (1.5 ml) was added NaH (60% dispersion in mineral oil, 8.8 mg, 0.22 mmol) and the resulting mixture was stirred at room temperature under nitrogen for 15 min. Iodomethane (30 μl, 0.48 mmol) was added via syringe and the resulting mixture was stirred 20 min. Methanol (1 ml) was added and most of the solvent was removed in vacuo. The residue was dissolved in ethyl acetate and the organic phase was washed three times with a saturated aqueous sodium bicarbonate solution, dried (MgSO₄) and concentrated in vacuo. The residue was loaded onto an Isolute® SCX cartridge. The cartridge was eluted with methanol, then with a 1M solution of ammonia in methanol. The latter was concentrated in vacuo to give the title compound as a pale yellow foam. MS (ES+ve): [M+H]⁺ at m/z 404 (C₂₄H₂₅N₃O₃ requires [M+H]⁺ at m/z 404).

### EXAMPLE 131

### 1-(8-{[(1S,2S)-2-hydroxy-2,3-dihydro-1H-inden-1-yl]oxy}-2,3-dimethylimidazo[1,2-a]pyridin-6-yl)-2(1H)-pyridinone; and 1-(8-{[(1R,2R)-2-hydroxy-2,3-dihydro-1H-inden-1-yl]oxy}-2,3-dimethylimidazo[1,2-a]pyridin-6-yl)-2(1H)-pyridinone

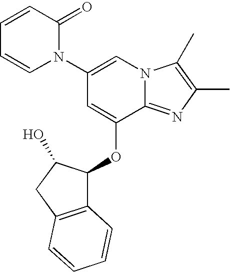

A mixture of 1-(8-hydroxy-2,3-dimethylimidazo[1,2-a]pyridin-6-yl)-2(1H)-pyridinone (50 mg, 0.20 mmol Description 23), 6,6a-dihydro-1aH-indeno[1,2-b]oxirene (39 mg, 0.30 mmol) and triethylamine (56 μl, 0.40 mmol) in methanol (2 ml) and water (0.2 ml) was reacted at room temperature for 3 days. The mixture was applied to an Isolute® SCX cartridge and eluted with methanol followed by 2M NH₃ in methanol. The basic fractions were combined and evaporated under reduced pressure. This residue was purified by column chromatography on silica eluting with 5-95 2M NH₃ in methanol-dichloromethane to afford the title compound MS (ES+ve): [M+H]⁺ at m/z 388 (C₂₃H₂₁N₃O₃ requires [M+H]⁺ at m/z 388).

### EXAMPLE 132

### 1-[8-(2,3-Dihydro-1H-inden-1-yloxy)-2,3-dimethylimidazo[1,2-a]pyridin-6-yl]-2(1H)-pyridinone

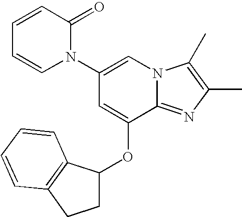

A mixture of 1-(8-hydroxy-2,3-dimethylimidazo[1,2-a]pyridin-6-yl)-2(1H)-pyridinone (75 mg, 0.29 mmol Description 23), 1-bromo-2,3-dihydro-1H-indene (116 mg, 0.58 mmol; Journal of the American Chemical Society (2005), 127(30), 10482-10483.) and potassium carbonate (51 mg, 0.37 mmol) in dimethylformamide (3 ml) was stirred at room temperature for 18 hours. The mixture was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane. Fractions containing the product were combined and evaporated under reduced pressure. The residue was dissolved in 1-1 acetonitrile and water and freeze dried overnight to afford the title compound MS (ES+ve): [M+H]⁺ at m/z 372 (C₂₃H₂₁N₃O₂ requires [M+H]⁺ at m/z 372).

### EXAMPLE 133

### 1-[2,3-Dimethyl-8-(1,2,3,4-tetrahydro-1-naphthalenyloxy)imidazo[1,2-a]pyridin-6-yl]-2(1H)-pyridinone

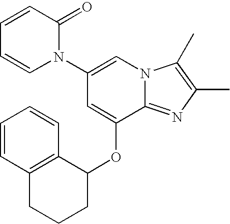

This compound can be prepared from 1-(8-hydroxy-2,3-dimethylimidazo[1,2-a]pyridin-6-yl)-2(1H)-pyridinone (Description 23) using the same method as for 1-[8-(2,3-Dihydro-1H-inden-1-yloxy)-2,3-dimethylimidazo[1,2-a]pyridin-6-yl]-2(1H)-pyridinone) (Example 132), using 1-bromo-1,2,3,4-tetrahydronaphthalene (Tetrahedron (1989), 45(24), 7869-78.) MS (ES+ve): [M+H]⁺ at m/z 386 (C₂₄H₂₃N₃O₂ requires [M+H]⁺ at m/z 386).

### EXAMPLE 134

### 1-(8-{[(2-Ethylphenyl)methyl]oxy}-2,3-dimethylimidazo[1,2-a]pyridin-6-yl)-2(1H)-pyridinone

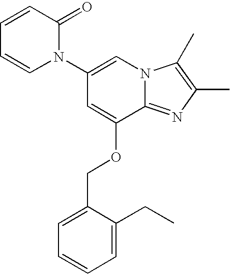

A mixture of 1-(8-hydroxy-2,3-dimethylimidazo[1,2-a]pyridin-6-yl)-2(1H)-pyridinone (50 mg, 0.20 mmol Description 23), 1-(bromomethyl)-2-ethylbenzene (65 mg, 0.29 mmol) and potassium carbonate (40 mg, 0.29 mmol) in dimethylformamide (2 ml) was stirred at room temperature overnight. The mixture was applied to an Isolute® SCX cartridge and eluted with methanol followed by 2M NH₃ in methanol. The basic fractions were combined and evaporated under reduced pressure. This residue was purified by column chromatography on silica eluting with 3-97% 2M NH₃ in methanol-dichloromethane to afford the title compound MS (ES+ve): [M+H]⁺ at m/z 374 (C₂₃H₂₃N₃O₂ requires [M+H]⁺ at m/z 374).

### EXAMPLE 135

### 1-[2,3-Dimethyl-8-({[2-(1-methylethyl)phenyl]methyl}oxy)imidazo[1,2-a]pyridin-6-yl]-2(1H)-pyridinone

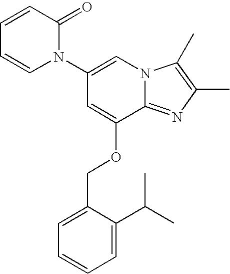

This compound can be prepared from 1-(8-hydroxy-2,3-dimethylimidazo[1,2-a]pyridin-6-yl)-2(1H)-pyridinone (Description 23) using the same method as for 1-(8-{[(2-ethylphenyl)methyl]oxy}-2,3-dimethylimidazo[1,2-a]pyridin-6-yl)-2(1H)-pyridinone (Example 134), substituting 1-(bromomethyl)-2-(1-methylethyl)benzene) for 1-(bromomethyl)-2-ethylbenzene MS (ES+ve): [M+H]⁺ at m/z 388 (C₂₄H₂₅N₃O₂ requires [M+H]⁺ at m/z 388).

### EXAMPLE 136

### Preparation of H⁺/K⁺ ATPase

The H⁺/K⁺ ATPase assay was based on Hongo et al (1990) Jpn J Pharmacol. 52.295-305 “Purification and characterization of (H+,K+)-ATPase from hog gastric mucosa”

Preparation of H+/K+ ATPase

Fresh porcine stomachs were obtained and washed with 0.9% NaCl. The surface mucus was removed by vigorously wiping; the findic mucosa was then removed from the underlying muscular layer and suspended in a chilled 0.25M sucrose solution. Homogenization was carried out with polytron setting 5 for 3 minutes and the homogenate was centrifugated at 8,000 rpm for 15 minutes. The supernatants after filtration over stainless gauze were then centrifugated at 13,000 rpm for 15 minutes. The resulting supernatants were recentrifuged using rotor type 70 Ti at 31,000 rpm for 1 hour to obtain the crude microsomal sediment (F0). The crude microsomes were suspended in the 0.25M sucrose solution. The resuspended microsomes (4 mL, 11 mg/mL) were layered on a single step gradient made from 5 mL of 7% (w/v) Ficoll in the 0.25 sucrose solution and centrifugated using rotor type 41 Ti at 30,000 rpm for 40 minutes. The light membrane (FB) appeared at the interface of the 7% Ficoll, and the heavy membrane (FS) appeared in the form of a sediment. FB was collected and diluted to 10-fold with the 0.25M sucrose solution and then centrifugated using rotor type 41 Ti at 31,000 rpm for 1 hour.

The resulting sediments were resuspended in the 0.25M sucrose solution by 10 strokes of a loose-fitting motor-driven, Telfon pestle rotating at 1,000 rpm in a homogenizer and refrigerated overnight for the final purification.

The resuspended microsomes (8 mL/3.5 mg/mL) were furthermore layered on top of 5 mL of 7% (w/v) Ficoll in the 0.25M sucrose solution and centrifuged using rotor type 41 at 30,000 rpm for 40 minutes.

The heavy membrane (FBS), appearing in the form of a sediment, was then resuspended in the 0.25M sucrose solution and centrifugated using rotor type 41 Ti at 37,500 rpm for 2 hours.

The pellet was resuspended in 0.25M sucrose solution and stored at −80° C. until use.

Alternatively the protein can be prepared in the following procedure:
1. 4 pig stomachs obtained fresh from abattoir.
2. Stomachs excised and washed thoroughly with cold water, then shipped on ice saturated in phosphate buffered saline solution (PBS).
3. All subsequent operations are performed at 4° C.; the harvesting of material from the stomachs should be performed within a cold room (steps 2-6).
4. Fundic regions are removed and rinsed twice in ice-cold PBS.
5. The mucosa is peeled away from the stomach wall using a scalpel (it will tear off relatively easily and stay intact).
6. Liberally apply ice-cold saturated NaCl to the surface, leaving the stomachs upside-down in the NaCl solution. Leave to soak around 15 minutes.
7. After soaking vigorously wipe the mucosae with paper towels to remove all mucus and loose surface cells
8. Using a spoon, scrape the mucosa away from the underlying connective tissue from all 4 stomachs, and put it in 800 mL of 0.25M sucrose containing Sigma Cocktail protease inhibitors, 1 mM EDTA, 1 mM EGTA pH 7.4.
9. Using scissors, roughly homogenize the tissue as much as possible.
10. Dounce homogenize the suspension using a tight-fitting Teflon-glass homogenizer (e.g. 50 mL volume) with 6 strokes at maximum rpm.
11. Centrifuge the homogenate for 45 minutes at 20,000 g in a Beckman JA18 rotor at 4 C. Chill the tubes and rotor beforehand in the cold room (same for all subsequent centrifugation steps).
12. Decant off the supernatant from this spin and centrifuge further for 45 minutes at 100,000 g in a Beckman JA30.5 rotor.
13. Resuspend the pellets in a total of 171 mL of 0.25M sucrose containing Sigma Cocktail protease inhibitors: dounce homogenize as above. Keep on ice, and pool in one vessel.
14. Prepare discontinuous (step) gradients in tubes for a Beckman JS24.38 rotor (swing out type), consisting of 15 mL of 7% Ficoll (w/w) overlaid onto 5 mL of 34% (w/v) sucrose. Load carefully 16 mL of sample on the top.
15. Being careful not to disturb the tubes, centrifuge them for 2 h at 100,000 g in a JA24.38 rotor.
16. After centrifugation, There should be a clearly visible cloudy region at the Ficoll/0.25M sucrose interface. Collect this using a 1 mL Gilson pipette (try not to take sucrose—the material will be in the Ficoll and you should be able to harvest it all without disturbing the interface. Keep on ice
17. Pool all the samples together in one tube. Fill with 0.25M sucrose, and centrifuge for 45 minutes at 100,000 g in a Beckman JA30.5 rotor. Discard carefully the supernatant and resuspend the pellet in approximately 1 mL of 5 mM Pipes/Tris pH 7.4.
18. Transfer the material to a round bottom flask and shell-freeze (rotate flask at an angle in a bath of methanol and cardice, to form a thin “shell” of frozen material on inside of flask). Lyophilize immediately overnight.
19. Re-suspend the lyophilized material in a minimum volume of Pipes/Tris and freeze aliquots down at —80 C.

### EXAMPLE 137

### H+/K+ ATPase assay

The H+/K+ ATPase activity was determined by spectrophotometric quantification of enzymatic inorganic phosphate release from ATP. Concentration response curve experiments were carried out from a starting concentration of test compounds of 100 μM with serial half log units dilution to 3 nM. One full curve contains 8 points in duplicate.

a) for determination of total ATPase activity 1 μL of the test compound was preincubated in 80 μL incubation assay buffer (37.5 mM Bis-Tris acetate, pH 5.5, 4 mM MgCl₂, 10 mM KCl) and H+/K+ ATPase enzyme from example 68 (10 μL of 0.25 μg/mLmL) at 37° C. for 15 minutes.

b) for non-specific ATPase activity 1 μL of the test compound was preincubated in 80 μl control assay buffer (37.5 mM Bis-Tris acetate, pH 5.5, 4 mM MgCl₂) and H+/K+ ATPase enzyme from example 14 (10 μL of 0.25 μg/mLmL) at 37° C. for 15 minutes. The reaction was initiated by adding 10 μL of 1 mM ATP to (a) and (b) and then incubating at 37° C. for 60 minutes.

Malachite green buffer was added 100 μL/well and absorbance was red at 630 nm. Specific H+/K+ ATPase activity is the total ATPase activity (in the presence of 10 mM KCl: reaction (a)) minus the basal, non-specific, ATPase activity (in the absence of KCl: reaction (b)).

Alternatively, the assay can be performed with the following slightly modified procedure:

Concentration response curve experiments were carried out from a starting concentration of test compounds of 100 μM with serial 4-fold dilutions. One full curve contains 11 points in duplicate.

a) for determination of total ATPase activity 0.1 μL of the test compound was preincubated in 10 μL incubation assay buffer (20 mM PIPES, pH 6.0, 1 mM MgCl2, 10 mM KCl) and H+/K+ ATPase enzyme from example 14 (final assay concentration 0.25 μg/mL) at 37° C. for 15 minutes.

b) for non-specific ATPase activity 0.1 μl of the test compound was preincubated in 10 μl control assay buffer (20 mM PIPES, pH 6.0, 1 mM MgCl2) and H+/K+ ATPase enzyme from example 14 (final assay concentration 0.25 μg/mL) at 370 C for 15 minutes.

The reaction was initiated by adding 10 μL of 0.2 mM ATP to (a) and (b) and then incubating at 37° C. for 60 minutes.

Malachite green buffer was added 30 μl/well and absorbance was red at 630 nm. Specific H+/K+ ATPase activity is the total ATPase activity (in the presence of 10 mM KCl: reaction (a)) minus the basal, non-specific, ATPase activity (in the absence of KCl: reaction (b)).

All compounds tested in one or other of the two methods described above had an IC₅₀ of ≦10 uM.

1. A compound of formula (I) 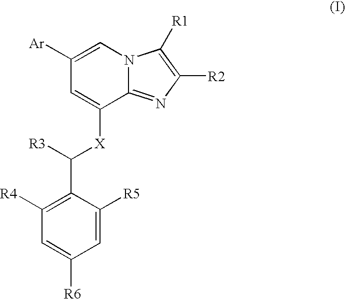
wherein:
X is NH, NR7 or O;
R1 is H, C₁₋₄ alkyl, CH₂CN, CH₂NH₂, C₃₋₆cycloalkyl, C₃₋₆cycloalkylC₁₋₄alkyl, C₁₋₄alkoxy, C₂₋₆alkenyl, C₂₋₆alkenyloxyC₁₋₄alkyl, C₂₋₆alkynyl, hydroxyC₁₋₄alkyl, C₁₋₄alkoxyC₁₋₄alkyl, hydroxyC₁₋₄alkoxyC₁₋₄alkyl, fluoroC₁₋₄alkyl, C₂₋₆alkynyloxyC₁₋₄alkyl, C₁₋₄alkylsulphonylC₁₋₄alkyl or NR8R9, where each of R8 and R9, which may be the same or different, are H or C₁₋₄alkyl or, together with the nitrogen to which they are attached, form a 5- or 6-membered heterocyclic group containing 0 to 3 further heteroatoms selected from N, O and S;
R2 is C₁₋₄alkyl, NH_{2,} C₃₋₆cycloalkyl, C₃₋₆cycloalkylC₁₋₄alkyl, C₁₋₄alkoxy, C₂₋₆alkenyl, hydroxyC₁₋₄alkyl, C₁₋₄alkoxyC₁₋₄alkyl, hydroxyC₁₋₄alkoxyC₁₋₄alkyl, cyanoC₁₋₄alkyl, haloC₁₋₄alkyl or aminocarboxyC₁₋₄alkyl;
R3 is H or C₁₋₄alkyl;
R4 and R5, which may be the same or different, are H, C₁₋₄alkyl, OH, halogen, C₁₋₄alkoxy, NR14R15 where each of R14 and R15, which may be the same or different, are H or C₁₋₄alkyl, NHCONR10R11 or OCONR10R11 where each of R10 and R11, which may be the same or different, are H or C₁₋₄alkyl or, together with the nitrogen to which they are attached, form a 5- or 6-membered heterocyclic group containing 0 to 3 further heteroatoms selected from N, O and S;
or R3 and R4 together with the interconnecting atoms form a 5- or 6-membered carbocyclic group or a heterocyclic group containing 1 heteroatom selected from N, O and S, which carbocyclic or heterocyclic group is optionally substituted with one group selected from the group consisting of C₁₋₄alkyl, OH, OC₁₋₄alkyl, halogen and NR16R17 where each of R16 and R17, which may be the same or different, are H or C₁₋₄alkyl;
R6 is H, C₁₋₄alkyl, halogen, OH, NHCO₂C₁₋₄alkyl, NR18R19 where each of R18 and R19, which may be the same or different, are H or C₁₋₄alkyl;
R7 is C₁₋₄alkyl;
or R4 and R7 together with the interconnecting atoms form a 5- to 7-membered heterocyclic group containing 0 or 1 further heteroatoms selected from N, O and S, which heterocyclic group is optionally substituted with one group selected from the group consisting of C₁₋₄alkyl, OH, OC₁₋₄alkyl, halogen and NR20R21 where each of R20 and R21, which may be the same or different, are H or C₁₋₄alkyl; and
Ar is aryl, or a 5- or 6-membered monocyclic, or a 7- to 12-membered bicyclic, heteroaryl group (containing 1 to 4 heteroatoms selected from N, O and S) which aryl or heteroaryl group is independently optionally substituted with 1, 2, 3 or 4 groups selected from the group consisting of halogen, hydroxy, oxo, cyano, C₁₋₄alkyl, C₁₋₄alkoxy, hydroxyC₁₋₄alkyl, hydroxyC₁₋₄alkoxy, haloC₁₋₄alkyl, haloC₁₋₄alkoxy, arylC₁₋₄alkoxy, C₁₋₄alkylthio, C₁₋₄alkoxyC₁₋₄alkyl, C₃₋₆cycloalkylC₁₋₄alkoxy, C₁₋₄alkanoyl, C₁₋₄alkylsulfonyl, C₁₋₄alkylsulfonyloxy, C₁₋₄alkylsulfonylC₁₋₄alkyl, arylsulfonyl, arylsulfonyloxy, aryloxysulfonyl, arylsulfonylC₁₋₄alkyl, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl, arylcarboxamidoC₁₋₄alkyl, arylaminosulfonyl, arylaminocarbonyl, aroyl, aroylC₁₋₄alkyl, arylC₁₋₄alkanoyl, NR12R13 and CONR12R13, where each of R12 and R13, which may be the same or different, are H, C₁₋₄ alkyl, hydroxyC₁₋₄alkyl, alkoxyC₁₋₄alkyl, C₃₋₆cycloalkyl, C₃₋₆cycloalkylC₁₋₄alkyl or, together with the nitrogen to which they are attached, form a 5- or 6-membered heterocyclic group containing a further heteroatom selected from N, O or S;
or a pharmaceutically acceptable salt thereof;
with the proviso that the compound is not: 1-(8-{[(4-fluorophenyl)methyl]oxy}-2-methylimidazo[1,2-a]pyridin-6-yl)-2(1H)-pyridinone.

2-4. (canceled)

5. A compound according to claim 1 wherein Ar is selected from phenyl, pyridinyl, pyrimidinyl, pyridazinyl, pyrazinyl, oxazolyl, furanyl, oxadiazolyl, isoxazolyl, pyrazolyl, triazolyl, tetrazolyl, imidazolyl, thienyl, methylpyridinyl, methyloxypyridinyl, carboxamidopyridinyl, aminopyridinyl, pyridinonyl, methylpyridinonyl cyanopyridinonyl, chloropyridinonyl, pyrazolonyl, methylpyrazolyl, hydroxymethylpyrazolyl, pyrimidinonyl, pyrimidinedionyl, dimethylpyrimidinedionyl and pyridazinonyl.

6. A compound according to claim 1 wherein the optional substituents for Ar are C₁₋₄alkyl, C₁₋₄alkoxy, carboxamide, oxo, cyano, amino or hydroxyC₁₋₄alkyl.

7-9. (canceled)

10. A compound according to claim 1 wherein R1 is C₁₋₄ alkyl, CH₂CN, C₁₋₄alkoxy, hydroxyC₁₋₄alkyl or C₁₋₄alkoxyC₁₋₄alkyl.

11. A compound according to claim 1 wherein R2 is C₁₋₄alkyl.

12. A compound according to claim 11 wherein R1 and R2 are both methyl.

13. A compound according to claim 1 wherein R3 is H.

14. A compound according to claim 1 wherein R4 is H, C₁₋₄alkyl or halogen.

15. A compound according to claim 1 wherein R5 is H, C₁₋₄alkyl or halogen.

16. A compound according to claim 15 wherein R4 and R5 are not both H.

17. A compound according to claim 1 wherein R6 is H, C₁₋₄alkyl or halogen.

18. A compound according to claim 17 wherein R4 and R5 are both methyl and R6 is H.

19. A compound according to claim 17 wherein R4 is methyl, R5 is ethyl and R6 is H.

20. (canceled)

21. A compound according to claim 1 wherein X is NH or O; R3 is H; R1, R2, R4 and R5 are all methyl; R6 is H; and Ar is pyridinonyl, pyrazolyl or triazolyl.

22. A compound according to claim 17 wherein R6 is fluoro.

23. A compound according to claim 16 wherein one of R4 and R5 is fluoro, chloro or bromo and the other is H.

24. A compound according to claim 16 wherein R4 and R5 are both fluoro.

25. (canceled)

26. A compound according to claim 1 wherein X is NH or O; R3 is H; R1 and R2 are both methyl; R6 is H or fluoro; one of R4 and R5 is chloro and the other is H; and Ar is pyridinonyl, pyrazolyl or triazolyl.

27. (canceled)

28. A compound according to claim 1 wherein X is NH or O; R3 is H; R1 and R2 are both methyl; R6 is fluoro; R4 and R5 are both methyl; and Ar is pyridinonyl, pyrazolyl or triazolyl.

29. A compound according to claim 1 which is the compound of any one of examples 1 to 135.

30-40. (canceled)
